(19) United States
(12) Patent Application Publication
(10) Pub. No.: US 2024/0011031 A1
(43) Pub. Date: Jan. 11, 2024

(54) COMPOSITIONS COMPRISING A NUCLEASE AND USES THEREOF

(71) Applicant: Arbor Biotechnologies, Inc., Cambridge, MA (US)

(72) Inventors: David A. Scott, Cambridge, MA (US); David R. Cheng, Boston, MA (US); Winston X. Yan, Boston, MA (US); Tia M. Ditommaso, Waltham, MA (US)

(73) Assignee: Arbor Biotechnologies, Inc., Cambridge, MA (US)

(21) Appl. No.: 18/021,739

(22) PCT Filed: Aug. 17, 2021
(86) PCT No.: PCT/US2021/046360

§ 371 Date: Feb. 16, 2023

Related U.S. Application Data
(60) Provisional application No. 63/066,669, filed on Aug. 17, 2020.

(51) Int. Cl. C12N 15/113; C12N 9/22; C12N 15/86

(52) U.S. Cl. CPC C12N 15/113; C12N 9/22; C12N 15/86; C12N 2310/20

(57) ABSTRACT

The present invention relates nucleases, processes for characterizing the nucleases, compositions comprising the nucleases, and methods of using the nucleases.

### CROSS REFERENCE TO RELATED APPLICATION

This application claims the benefit of U.S. Provisional Application No. 63/066669, filed Aug. 17, 2020. The contents of the aforementioned application is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 16, 2021, is named A2186-7039WO_SL.txt and is 275,996 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

### SUMMARY OF THE INVENTION

It is against the above background that the present invention provides certain advantages and advancements over the prior art.

Although this invention disclosed herein is not limited to specific advantages or functionalities, the invention provides composition comprising: (a) a nuclease or a nucleic acid encoding the nuclease, wherein the nuclease comprises an amino acid sequence with at least 80% identity to any one of SEQ ID NOs: 1-37 and SEQ ID NOs: 221-224; and (b) an RNA guide or a nucleic acid encoding the RNA guide, wherein the RNA guide comprises a direct repeat sequence and a spacer sequence, wherein the nuclease binds to the RNA guide, and wherein the spacer sequence binds to a target nucleic acid.

In various aspects, the nuclease comprises a RuvC domain or a split RuvC domain.

In some aspects, the nuclease comprises a catalytic residue (e.g., aspartic acid or glutamic acid).

In some aspects, the nuclease comprises one or more of the following sequences: (a)

X₁X₂X₃X₄GX₅X₆ (SEQ ID NO: 233), wherein X₁ is V or A or C, X₂ is Y or F, X₃ is K or Q, X₄ is Y or F, X₅ is L or A or M or C or T, and X₆ is I or V or L; (b) LX₁NX₂ LV (SEQ ID NO: 234), wherein X₁ is W or K or R and X₂ is N or T or K or S or D or Q; (c) FDX₁X₂ G (SEQ ID NO: 235), wherein X₁ is G or Y and X₂ is T or S or M; (d) X₁ X₂ HR X₃X₄ P (SEQ ID NO: 236), wherein X₁ is I or L or V, X₂ is Y or L or M or F, X₃ is P or H or D or E, and X₄ is L or I or V or M; (e) GX₁ DX₂ GX₃ R (SEQ ID NO: 237), wherein X₁ is I or L or V, X₂ is I or V or L, and X₃ is F or Y; (f) RX₁ X₂ X₃ YR (SEQ ID NO: 238), wherein X₁ is K or Q or E, X₂ is H or D or E, and X₃ is F or V or L or I; and (g) X₁ DX₂ DX₃ NAAX₄ N (SEQ ID NO: 239), wherein X₁ is H or Y, X₂ is R or Q or V, X₃ is E or T or I or H or K or Q or D, and X₄ is N or R or I or V or K.

In some aspects, the nuclease comprises an amino acid sequence with at least 95% identity to any one of SEQ ID NOs: 1-37 and SEQ ID NOs: 221-224.

In some aspects, the nuclease comprises an amino acid sequence set forth in any one of SEQ ID NOs: 1-37 and SEQ ID NOs: 221-224.

In some aspects, the composition does not include a tracrRNA.

In some aspects, the direct repeat sequence comprises one or more of the following sequences: (a)

X₁X₂CCCTX₃ (SEQ ID NO: 240), wherein X₁ is G or A, X₂ is A or C, and X₃ is G or A; and (b)

X₁GGGX₂X₃X₄X₅X₆A (SEQ ID NO: 241), wherein X₁ is T or G, X₂ is T or G, X₃ is T or G, X₄ is A or G, X₅ is T or A, and X₆ is A or G or C.

In some aspects, the direct repeat sequence comprises a nucleotide sequence with at least 95% sequence identity to any one of SEQ ID NOs: 38-126 or 243-250.

In some aspects, the direct repeat sequence comprises the nucleotide sequence set forth in any one of SEQ ID NOs: 38-126 or 243-250.

In some aspects, the nuclease comprises an amino acid sequence with at least 95% identity to SEQ ID NO: 2. In some aspects, the nuclease comprises the amino acid sequence set forth in SEQ ID NO: 2. In some aspects, the direct repeat sequence comprises a nucleotide sequence with at least 95% sequence identity to SEQ ID NO: 40 or SEQ ID NO: 41. In some aspects, the direct repeat sequence comprises a nucleotide sequence set forth in SEQ ID NO: 40 or SEQ ID NO: 41. In some aspects, the direct repeat sequence comprises a nucleotide sequence with at least 95% sequence identity to SEQ ID NO: 111 or 243.

In some aspects, the spacer sequence comprises between 15 and 24 nucleotides in length. In some aspects, the spacer sequence comprises about 19 or 20 nucleotides in length.

In some aspects, the target nucleic acid comprises a sequence complementary to a nucleotide sequence in the spacer sequence.

In some aspects, the target nucleic acid is adjacent to a protospacer adjacent motif (PAM) sequence, wherein the PAM sequence comprises a nucleotide sequence set forth as 5′-CN-3′, 5′-CCN-3′, 5′-NCN-3′, 5′-NCCN-3′, or 5′-NNCN-3′, wherein “N” is any nucleobase. In some aspects, the PAM sequence comprises a nucleotide sequence set forth as 5′-ACCN-3′, 5′-DCCN-3′, 5′-DTTN-3′, 5′-DYYN-3′, 5′-GCCN-3′, 5′-GTTN-3′, 5′-GYYN-3′, 5′-HCN-3′, 5′-HNCN-3′, 5′-HNCR-3′, 5′-HNCV-3′, 5′-RCCN-3′, 5′-RYCN-3′, 5′-TNCN-3′, wherein “D” is A or G or T, “H” A or C or T, “N” is any nucleobase, “R” is A or G, “V” is A or C or G, and “Y” is C or T. In some aspects, the PAM sequence comprises a nucleotide sequence set forth as 5′-CCA-3′, 5′-CCC-3′, 5′-CCT-3′, 5′-CCG-3′, 5′-ACCG-3′, 5′-CCCA-3′, 5′-CCCG-3′, 5′-TCCA-3′, or 5′-TCCT-3′.

In some aspects, the nuclease cleaves the target nucleic acid.

In some aspects, the target nucleic acid is single-stranded DNA or double-stranded DNA.

In some aspects, the composition comprises at least 10% greater enzymatic activity than a reference composition, e.g., at least 10% greater nuclease activity than a nuclease activity of a reference composition.

In some aspects, the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some aspects, the nucleic acid encoding the nuclease is codon-optimized for expression in a cell.

In some aspects, the nucleic acid encoding the nuclease is operably linked to a promoter.

In some aspects, the nucleic acid encoding the nuclease is in a vector. In some aspects, the vector comprises a retroviral vector, a lentiviral vector, a phage vector, an adenoviral vector, an adeno-associated vector, or a herpes simplex vector.

In some aspects, the composition is present in a delivery vehicle comprising a nanoparticle, a liposome, an exosome, a microvesicle, or a gene-gun.

The invention further provides a cell comprising the composition of the present invention.

In various aspects, the cell is a eukaryotic cell or a prokaryotic cell. In some aspects, the cell is a mammalian cell or a plant cell. In some aspects, the cell is a human cell.

The invention further provides a method of binding the composition of the present invention to the target nucleic acid in a cell comprising: (a) providing the composition; and (b) delivering the composition to the cell, wherein the cell comprises the target nucleic acid, wherein the nuclease binds to the RNA guide, and wherein the spacer sequence binds to the target nucleic acid.

In some embodiments, the nuclease comprises a RuvC domain or a split RuvC domain

In certain embodiments, the nuclease comprises a catalytic residue (e.g., aspartic acid or glutamic acid).

In some embodiments, the nuclease comprises one or more of the following sequences:
(a) X₁X₂X₃X₄GX₅X₆ (SEQ ID NO: 233), wherein X₁ is V or A or C, X₂ is Y or F, X₃ is K or Q, X₄ is Y or F, X₅ is L or A or M or C or T, and X₆ is I or V or L;
(b) LX₁NX₂LV (SEQ ID NO: 234), wherein X₁ is W or K or R and X₂ is N or T or K or S or D or Q;
(c) FDX₁X₂G (SEQ ID NO: 235), wherein X₁ is G or Y and X₂ is T or S or M;
(d) X₁X₂HR X₃X₄P (SEQ ID NO: 236), wherein X₁ is I or L or V, X₂ is Y or L or M or F, X₃ is P or H or D or E, and X₄ is L or I or V or M;
(e) GX₁DX₂GX₃R (SEQ ID NO: 237), wherein X₁ is I or L or V, X₂ is I or V or L, and X₃ is F or Y;
(f) RX₁X₂X₃YR (SEQ ID NO: 238), wherein X₁ is K or Q or E, X₂ is H or D or E, and X₃ is F or V or L or I; and
(g) X₁DX₂DX₃NAAX₄N (SEQ ID NO: 239), wherein X₁ is H or Y, X₂ is R or Q or V, X₃ is E or T or I or H or K or Q or D, and X₄ is N or R or I or V or K.

In certain embodiments, the nuclease comprises an amino acid sequence with at least 95% identity to SEQ ID NO: 2.

In some embodiments, the nuclease comprises the amino acid sequence set forth in SEQ ID NO: 2.

In certain embodiments, the composition does not include a tracrRNA.

In some embodiments, the direct repeat sequence comprises one or more of the following sequences:
(a) X₁X₂CCCTX₃ (SEQ ID NO: 240), wherein X₁ is G or A, X₂ is A or C, and X₃ is G or A; and
(b) X₁GGGX₂X₃X₄X₅X₆A (SEQ ID NO: 241), wherein X₁ is T or G, X₂ is T or G, X₃ is T or G, X₄ is A or G, X₅ is T or A, and X₆ is A or G or C.

In certain embodiments, the direct repeat sequence comprises a nucleotide sequence with at least 95% sequence identity to SEQ ID NO: 40 or SEQ ID NO: 41. In certain embodiments, the direct repeat sequence comprises a nucleotide sequence set forth in SEQ ID NO: 40 or SEQ ID NO: 41. In some embodiments, the direct repeat sequence comprises a nucleotide sequence with at least 95% sequence identity to SEQ ID NO: 111 or 243. In some embodiments, the direct repeat sequence comprises the nucleotide sequence set forth in SEQ ID NO: 111 or 243.

In certain embodiments, the spacer sequence comprises between 15 and 24 nucleotides in length. In some embodiments, the spacer sequence comprises about 19 or 20 nucleotides in length.

In some embodiments, the target nucleic acid comprises a sequence complementary to a nucleotide sequence in the spacer sequence.

In certain embodiments, the target nucleic acid is adjacent to a PAM sequence, wherein the PAM sequence comprises a nucleotide sequence set forth as 5′-CN-3′, 5′-CCN-3′, 5′-NCN-3′, 5′-NCCN-3′, or wherein “N” is any nucleobase. In some embodiments, the PAM sequence comprises a nucleotide sequence set forth as 5′-ACCN-3′, 5′-DCCN-3′, 5′-DTTN-3′, 5′-DYYN-3′, 5′-GCCN-3′, 5′-GTTN-3′, 5′-GYYN-3′, 5′-HCN-3′, 5′-HNCN-3′, 5′-HNCR-3′, 5′-HNCV-3′, 5′-RCCN-3′, 5′-RCCR-3′, 5′-TNCN-3′, wherein “D” is A or G or T, “H” A or C or T, “N” is any nucleobase, “R” is A or G, “V” is A or C or G, and “Y” is C or T. In certain embodiments, the PAM sequence comprises a nucleotide sequence set forth as 5′-CCA-3′, 5′-CCC-3′, 5′-CCT-3′, 5′-CCG-3′, 5′-ACCG-3′, 5′-CCCA-3′, 5′-CCCG-3′, 5′-TCCA-3′, or 5′-TCCT-3′.

In some embodiments, the nuclease cleaves the target nucleic acid.

In certain embodiments, the target nucleic acid is single-stranded DNA or double-stranded DNA.

In some embodiments, the composition comprises at least 10% greater enzymatic activity than a reference composition, e.g., at least 10% greater nuclease activity than a nuclease activity of a reference composition.

In certain embodiments, the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

In some embodiments, the nucleic acid encoding the nuclease is codon-optimized for expression in a cell.

In some embodiments, the nucleic acid encoding the nuclease is operably linked to a promoter.

In certain embodiments, the nucleic acid encoding the nuclease is in a vector. In certain embodiments, the vector comprises a retroviral vector, a lentiviral vector, a phage vector, an adenoviral vector, an adeno-associated vector, or a herpes simplex vector.

In some embodiments, the composition is present in a delivery vehicle comprising a nanoparticle, a liposome, an exosome, a microvesicle, or a gene-gun.

In one aspect, the disclosure provides a cell comprising the composition of any of the aspects or embodiments described herein. In some embodiments, the cell is a eukaryotic cell or a prokaryotic cell. In certain embodiments, the cell is a mammalian cell or a plant cell. In some embodiments, the cell is a human cell.

In another aspect, the disclosure provides a method of binding the composition of any one of the aspects or embodiments described herein to the target nucleic acid in a cell comprising:
(a) providing the composition; and
(b) delivering the composition to the cell,

wherein the cell comprises the target nucleic acid, wherein the nuclease binds to the RNA guide, and wherein the spacer sequence binds to the target nucleic acid.

### DEFINITIONS

The present invention will be described with respect to particular embodiments and with reference to certain figures, but the invention is not limited thereto but only by the claims. Terms as set forth hereinafter are generally to be understood in their common sense unless indicated otherwise.

As used herein, the term “catalytic residue” refers to an amino acid that activates catalysis. A catalytic residue is an amino acid that is involved (e.g., directly involved) in catalysis.

As used herein, the terms “domain” and “protein domain” refer to a distinct functional and/or structural unit of a polypeptide. In some embodiments, a domain may comprise a conserved amino acid sequence. As used herein, the term “RuvC domain” refers to a conserved domain or motif of amino acids having nuclease (e.g., endonuclease) activity. As used herein, a protein having a split RuvC domain refers to a protein having two or more RuvC motifs, at sequentially disparate sites within a sequence, that interact in a tertiary structure to form a RuvC domain

As used herein, the term “effector activity” refers to a biological activity. In some embodiments, effector activity includes enzymatic activity, e.g., catalytic ability of an effector. For example, effector activity can include nuclease activity.

As used herein, the term “nuclease” refers to an enzyme capable of cleaving a phosphodiester bond. A nuclease hydrolyzes phosphodiester bonds in a nucleic acid backbone. As used herein, the term “endonuclease” refers to an enzyme capable of cleaving a phosphodiester bond between nucleotides.

As used herein, the terms “parent,” “parent polypeptide,” and “parent sequence” refer to an original polypeptide (e.g., starting polypeptide) to which an alteration is made to produce a variant polypeptide of the present invention. In some embodiments, the parent is an effector having an identical amino acid sequence of the variant at one or more of specified positions. The parent may be a naturally occurring (wild-type) polypeptide. In a particular embodiment, the parent is an effector with at least 60%, at least 61%, at least 62%, at least 63%, at least 64%, at least 65%, at least 70%, at least 72%, at least 73%, at least 74%, at least 75%, at least 80%, at least 81%, at least 82%, at least 83%, at least 84%, at least 85%, at least 90%, at least 91%, at least 92%, at least 93%, at least 94%, at least 95%, at least 96%, at least 97%, at least 98%, at least 99% or 100% identity to a polypeptide of any one of SEQ ID NOs: 1-37 and SEQ ID NOs: 221-224.

As used herein, the term “protospacer adjacent motif” or “PAM” refers to a DNA sequence adjacent to a target sequence to which a complex comprising an effector and an RNA guide binds. In some embodiments, a PAM is required for enzyme activity. As used herein, the term “adjacent” includes instances in which an RNA guide of the complex specifically binds, interacts, or associates with a target sequence that is immediately adjacent to a PAM. In such instances, there are no nucleotides between the target sequence and the PAM. The term “adjacent” also includes instances in which there are a small number (e.g., 1, 2, 3, 4, or 5) of nucleotides between the target sequence, to which the targeting moiety binds, and the PAM.

As used herein, the terms “reference composition,” “reference sequence,” and “reference” refer to a control, such as a negative control or a parent (e.g., a parent sequence, a parent protein, a wild-type protein, or a complex comprising a parent sequence).

As used herein, the terms “RNA guide” or “RNA guide sequence” refer to any RNA molecule that facilitates the targeting of a polypeptide described herein to a target nucleic acid. For example, an RNA guide can be a molecule that recognizes (e.g., binds to) a target nucleic acid. An RNA guide may be designed to be complementary to a specific nucleic acid sequence. An RNA guide comprises a DNA targeting sequence and a direct repeat (DR) sequence. The terms CRISPR RNA (crRNA), pre-crRNA, mature crRNA, and gRNA are also used herein to refer to an RNA guide. As used herein, the term “pre-crRNA” refers to an unprocessed RNA molecule comprising a DR-spacer-DR sequence. As used herein, the term “mature crRNA” refers to a processed form of a pre-crRNA; a mature crRNA may comprise a DR-spacer sequence, wherein the DR is a truncated form of the DR of a pre-crRNA and/or the spacer is a truncated form of the spacer of a pre-crRNA.

As used herein, the term “targeting moiety” refers to a molecule or component (e.g., nucleic acid and/or RNA guide) that facilitates the targeting of another molecule or component to a target nucleic acid. In some embodiments, the targeting moiety specifically interacts or associates with the target nucleic acid.

As used herein, the term “substantially identical” refers to a sequence, polynucleotide, or polypeptide, that has a certain degree of identity to a reference sequence.

As used herein, the terms “target nucleic acid” and “target sequence” refer to a nucleic acid sequence to which a targeting moiety (e.g., RNA guide) specifically binds. In some embodiments, the DNA targeting sequence of an RNA guide binds to a target nucleic acid.

As used herein, the terms “trans-activating crRNA” and “tracrRNA” refer to an RNA molecule involved in or required for the binding of a targeting moiety (e.g., an RNA guide) to a target nucleic acid.

As used herein, the term “variant polypeptide” refers to a polypeptide comprising an alteration, e.g., a substitution, insertion, deletion and/or fusion, at one or more residue positions, compared to a parent polypeptide.

### BRIEF DESCRIPTION OF THE DRAWINGS

FIG. 1A, FIG. 1B, FIG. 1C, FIG. 1D, FIG. 1E, FIG. 1F, FIG. 1G, FIG. 1H, FIG. 1I, FIG. 1J, FIG. 1K, and FIG. 1L collectively show an alignment of the effectors of SEQ ID NOs: 1-37 and SEQ ID NOs: 221-224. The consensus sequence is shown at the top of the alignment.

FIG. 2 shows an alignment of the direct repeat sequences of SEQ ID NOs: 243, 243-245, 243, 246-248, 243, 249, 249, 249, 247, 243-244, and 250, respectively, in order of appearance. The consensus sequence is shown at the top of the alignment. The DNA version of the sequences is shown, and it is understood that a corresponding RNA version would typically include Us in place of Ts. The corresponding RNA versions are provided herein as SEQ ID Nos: 111-126.

FIG. 3A is a schematic showing generation of labeled dsDNA substrates for the dsDNA target cleavage experiments of Example 4.

FIG. 3B is a TBE-Urea denaturing gel showing cleavage of the spacer complementary strand of Target A (SEQ ID NO: 201) induced by a ribonucleoprotein (RNP) comprising the effector of SEQ ID NO: 2 and the mature crRNA of SEQ ID NO: 202.

FIG. 3C is a TBE-Urea denaturing gel showing cleavage of the non-spacer complementary strand of Target A (SEQ ID NO: 201) induced by an RNP comprising the effector of SEQ ID NO: 2 and the mature crRNA of SEQ ID NO: 202.

FIG. 3D is a control TBE-Urea denaturing gel showing that cleavage is not observed in the top strand of Non-Target B (SEQ ID NO: 204) using an RNP comprising the effector of SEQ ID NO: 2 and a mature crRNA designed to have complementarity to Target A.

FIG. 3E is a control TBE-Urea denaturing gel showing that cleavage is not observed in the bottom strand of Non-Target B (SEQ ID NO: 204) using an RNP comprising the effector of SEQ ID NO: 2 and a mature crRNA designed to have complementarity to Target A.

FIG. 4A is a schematic showing generation of labeled ssDNA substrates for the ssDNA target cleavage experiments of Example 5.

FIG. 4B is a TBE-Urea denaturing gel showing cleavage of single-stranded Target A (SEQ ID NO: 201) by an RNP comprising the effector of SEQ ID NO: 2 and the mature crRNA of SEQ ID NO: 202.

FIG. 4C is a control TBE-Urea denaturing gel showing that cleavage is not observed in the single-stranded Non-Target B (SEQ ID NO: 204) using an RNP comprising the effector of SEQ ID NO: 2 and a mature crRNA designed to have complementarity to Target A.

FIG. 5A is a schematic of the fluorescence depletion assay described in Example 6 to measure activity of the effector of SEQ ID NO: 2.

FIG. 5B shows plots of GFP Depletion Ratios (Non-target/target) for the effector of SEQ ID NO: 2 for Target 1 (SEQ ID NO: 208), Target 2 (SEQ ID NO: 210), Target 3 (SEQ ID NO: 212), Target 4 (SEQ ID NO: 214), and Target 5 (SEQ ID NO: 216). The Depletion Ratio values in FIG. 5B were calculated from measurements taken over a period of 12 hours.

FIG. 6 shows indels induced by the effector of SEQ ID NO: 2 at an AAVS1 target locus (SEQ ID 218) in HEK293 cells.

### DETAILED DESCRIPTION

The present disclosure relates to novel nucleases and methods of use thereof. In some aspects, a composition comprising a nuclease of the present invention having one or more characteristics is described herein. In some aspects, a method of producing a nuclease of the present invention is described. In some aspects, a method of delivering a composition comprising a nuclease of the present invention is described.

### Composition

In some aspects, the invention described herein comprises compositions comprising a nuclease. In some embodiments, a composition of the invention includes a nuclease, and the composition has nuclease activity. In some aspects, the invention described herein comprises compositions comprising a nuclease and a targeting moiety. In some embodiments, a composition of the invention includes a nuclease and an RNA guide sequence, and the RNA guide sequence directs the nuclease activity to a site-specific target. In some embodiments, a nuclease of the composition of the present invention is a recombinant nuclease.

In some embodiments, the composition described herein comprises an RNA-guided nuclease (e.g., a nuclease comprising multiple components). In some embodiments, a nuclease of the present invention comprises enzyme activity (e.g., a protein comprising a RuvC domain or a split RuvC domain) In some embodiments, the composition comprises a targeting moiety (e.g., an RNA guide). In some embodiments, the composition comprises a ribonucleoprotein (RNP) comprising a nuclease and a targeting moiety (e.g., RNA guide).

### Nuclease

In some embodiments, the composition of the present invention includes an effector (e.g., nuclease) described herein.

A nucleic acid sequence encoding a nuclease described herein may be substantially identical to a reference nucleic acid sequence if the nucleic acid encoding the nuclease comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, a nuclease described herein is encoded by a nucleic acid sequence having at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a reference nucleic acid sequence.

A nuclease described herein may substantially identical to a reference polypeptide if the nuclease comprises an amino acid sequence having at least about 60%, least about 65%, least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the reference polypeptide. The percent identity between two such polypeptides can be determined manually by inspection of the two optimally aligned polypeptide sequences or by using software programs or algorithms (e.g., BLAST, ALIGN, CLUSTAL) using standard parameters. One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative amino acid substitution or one or more conservative amino acid substitutions.

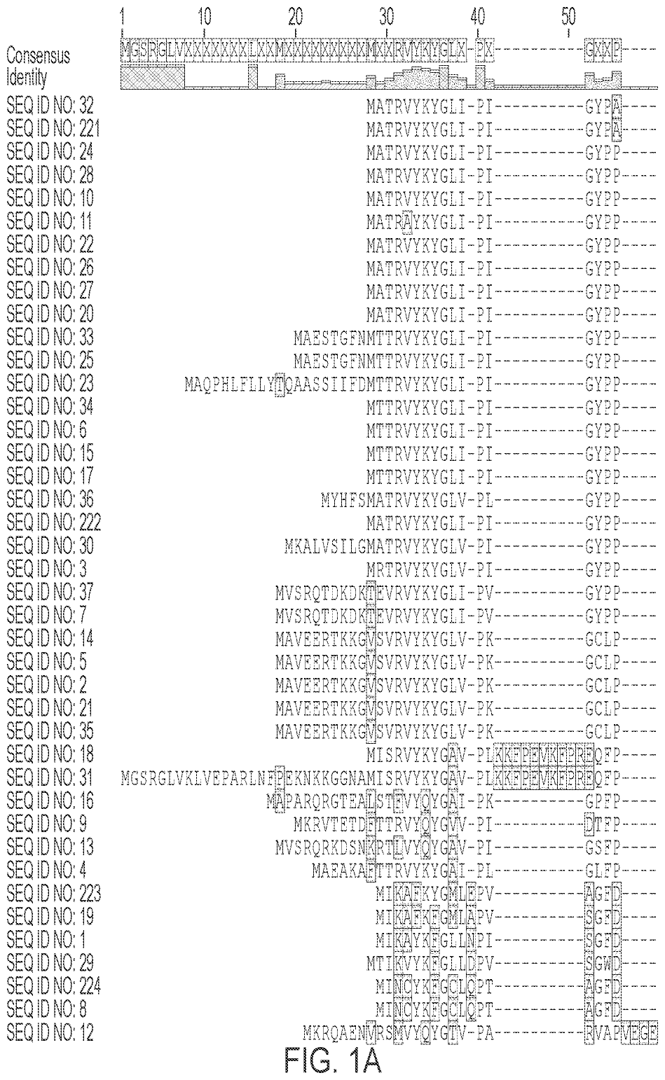

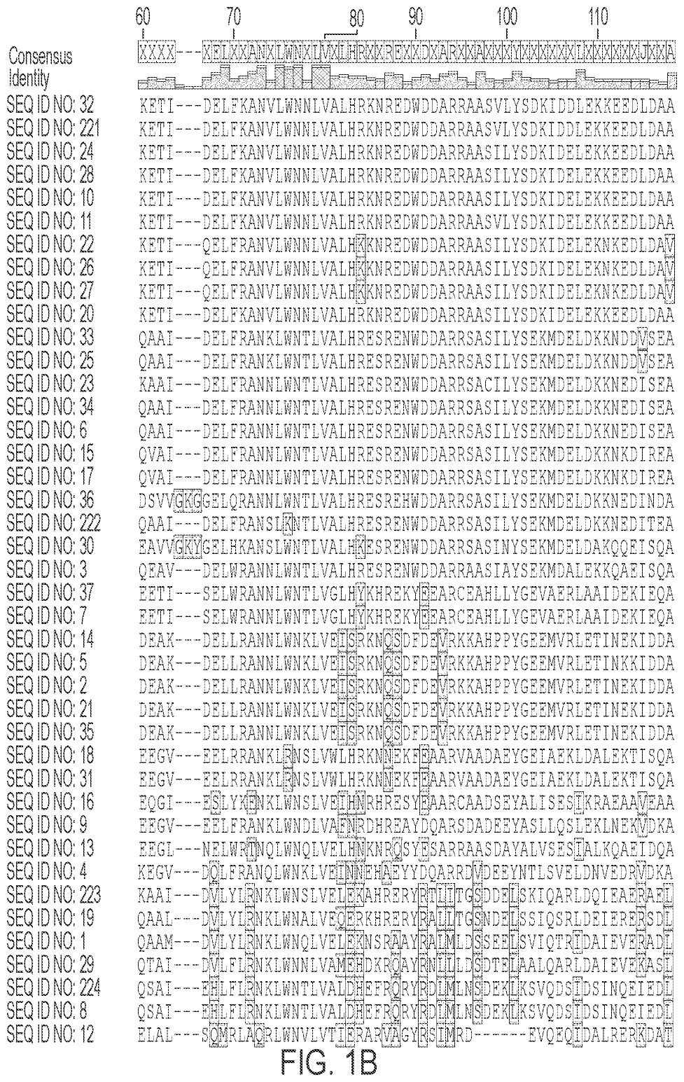

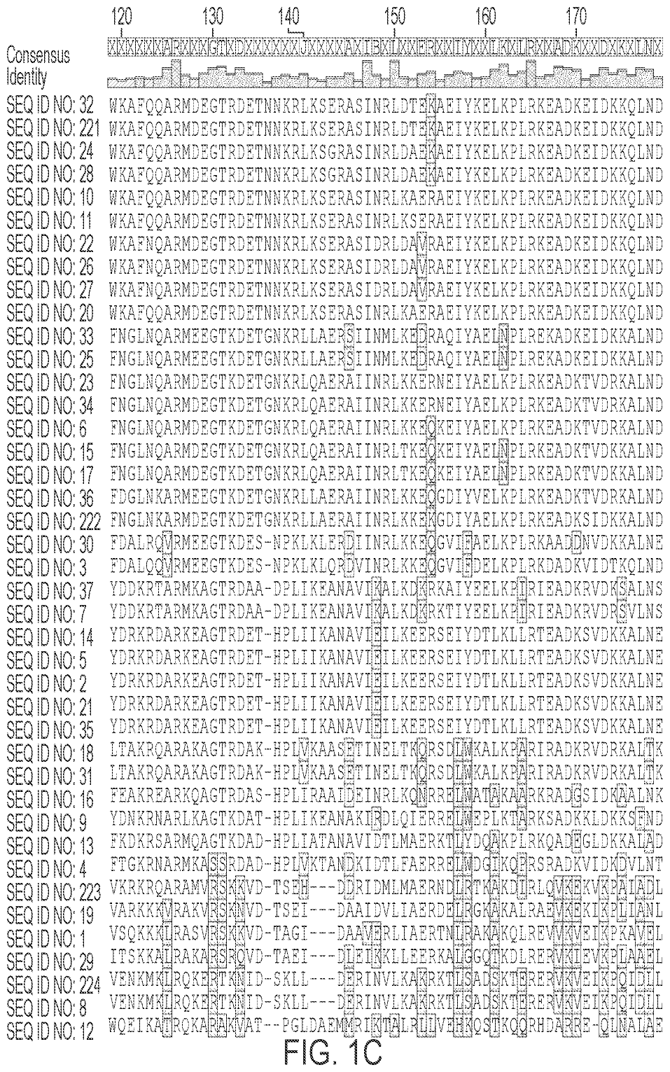

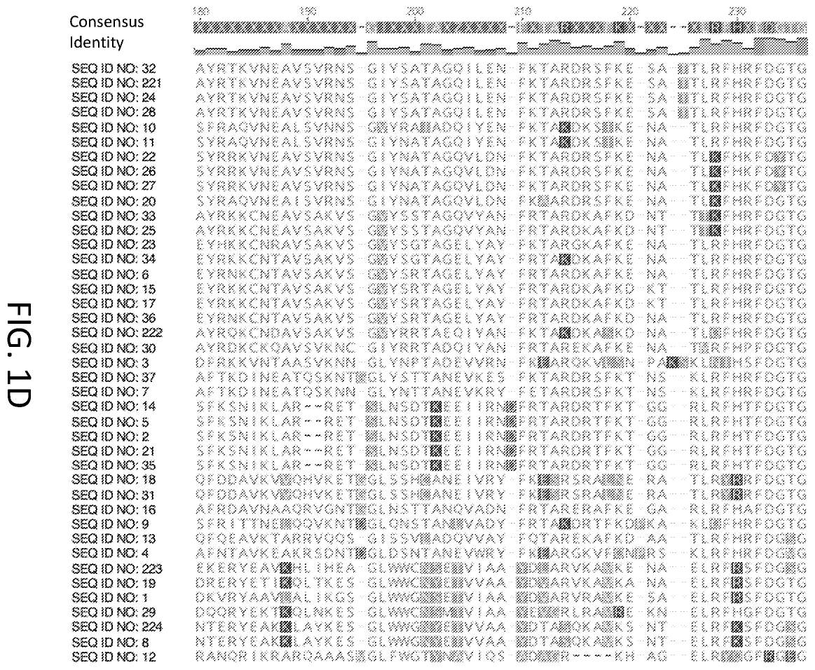

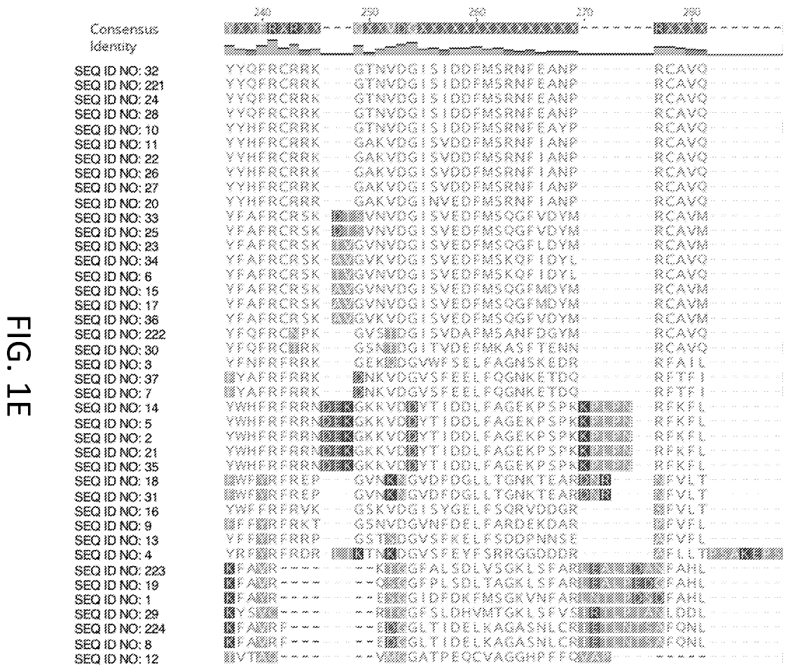

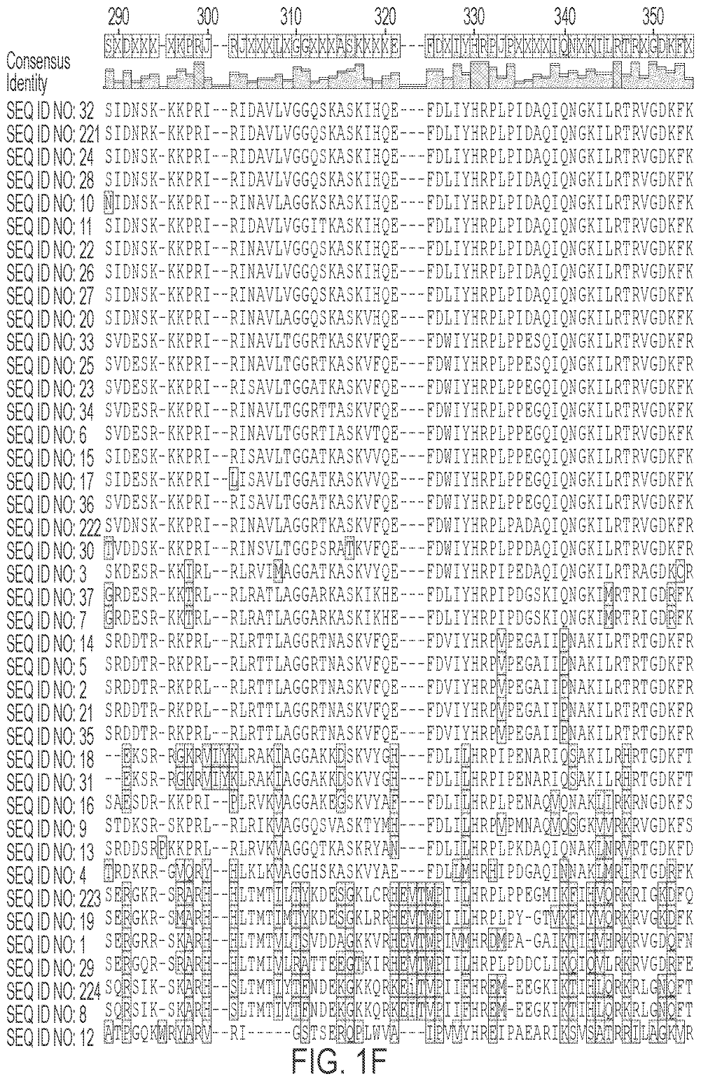

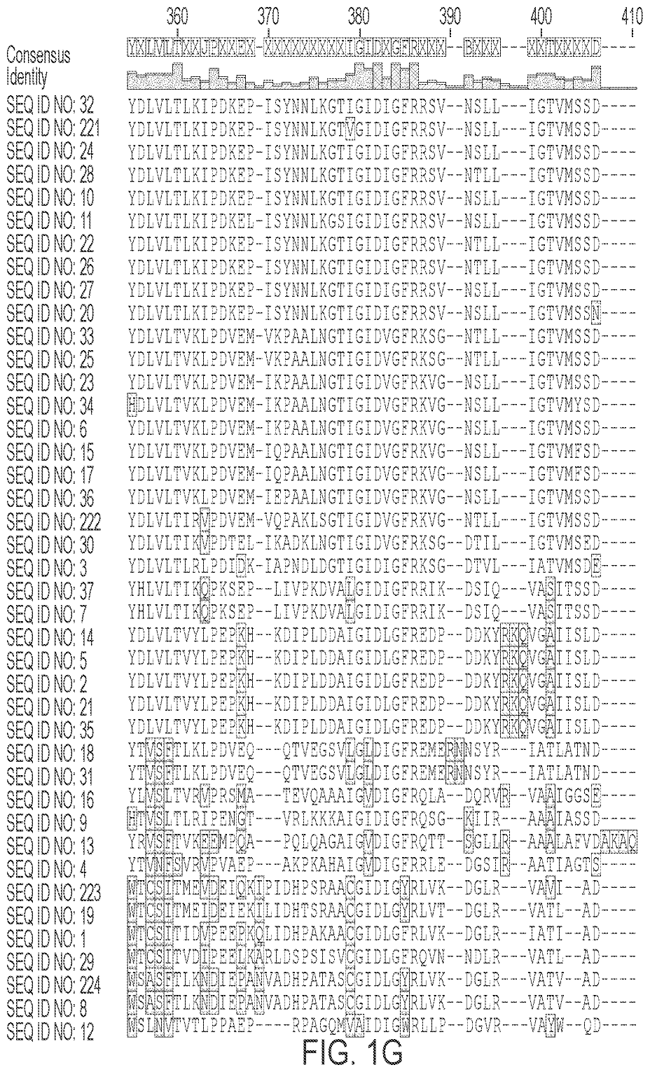

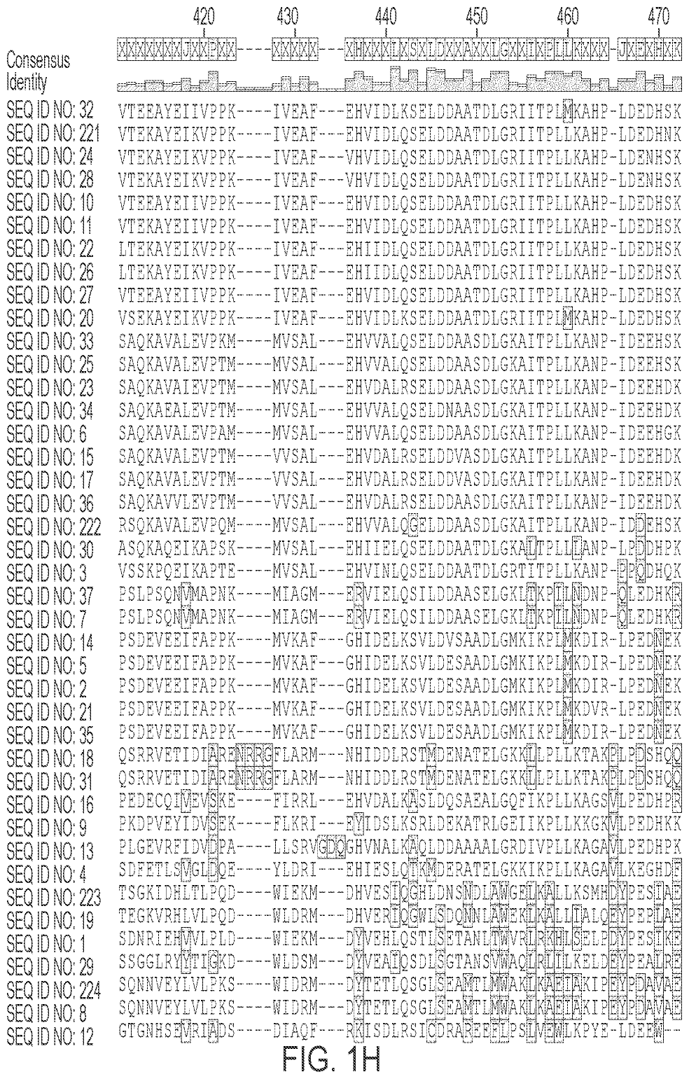

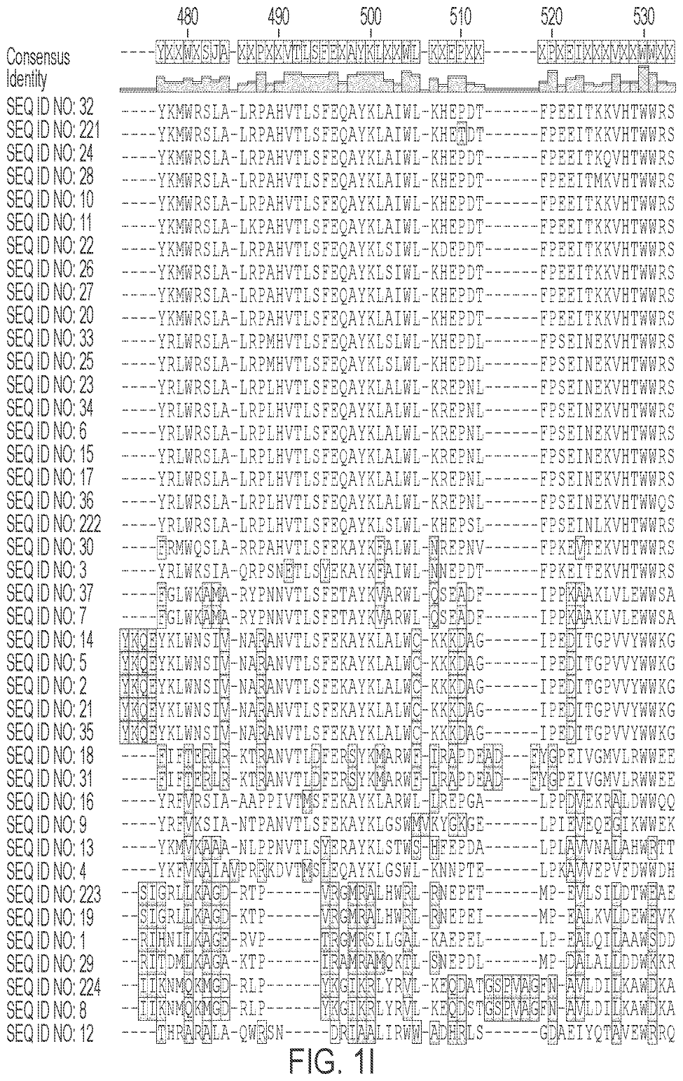

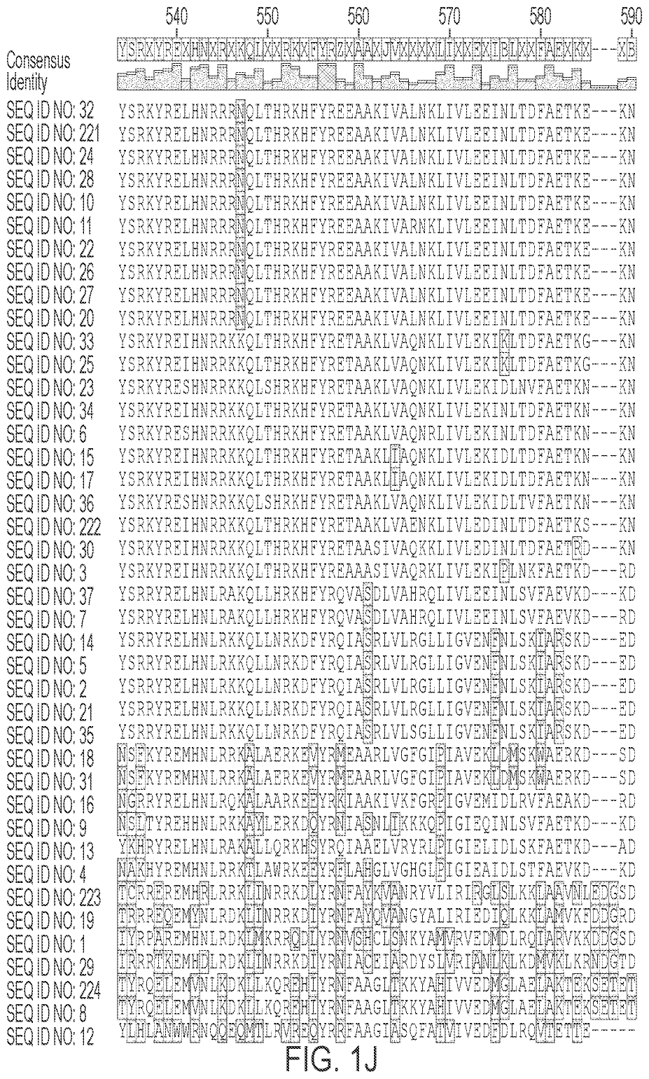

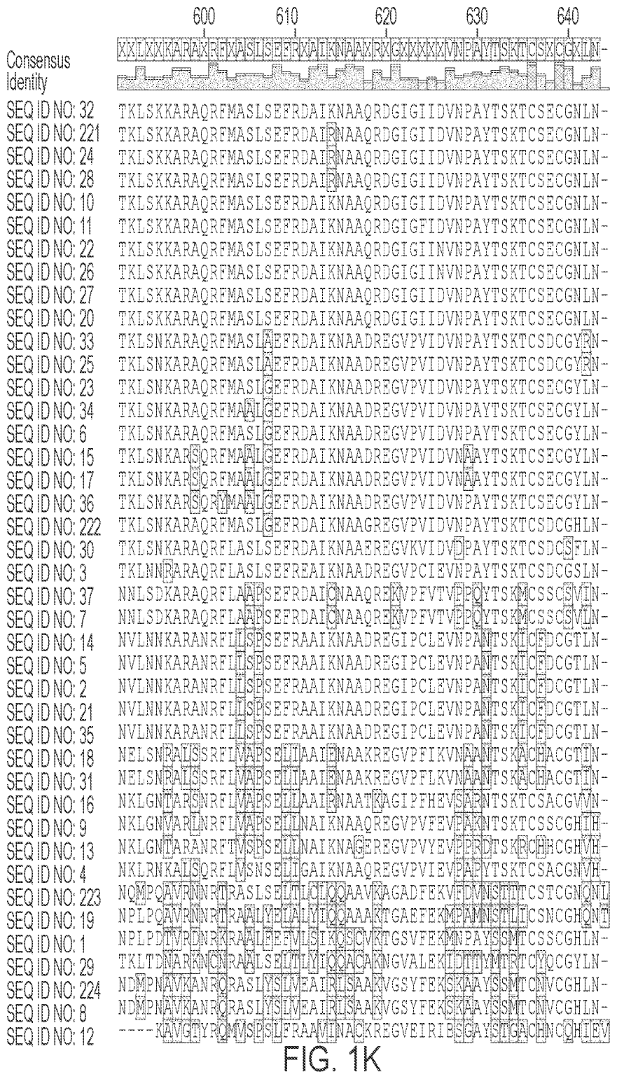

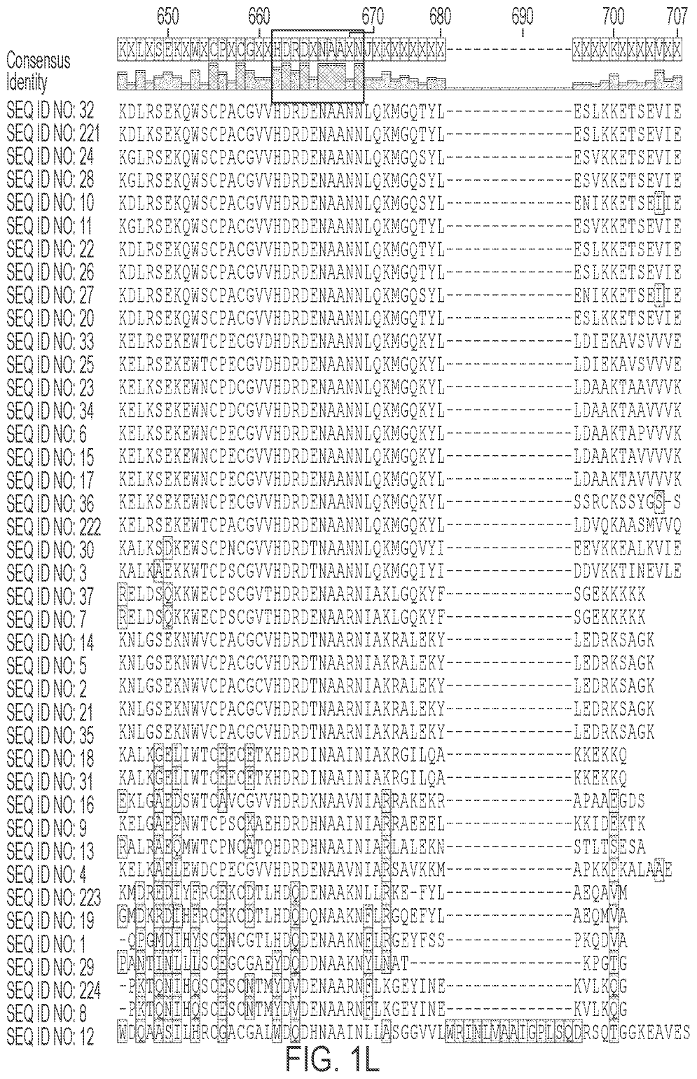

In some embodiments, a nuclease of the present invention comprises a polypeptide sequence having 60, 65, 70, 75, 80, 85, 90, 91, 92, 93, 94, 95, 96, 97, 98, 99 or 100% identity to any one of SEQ ID NOs: 1-37 and SEQ ID NOs: 221-224. In some embodiments, a nuclease of the present invention comprises a polypeptide sequence having greater than 50, 60, 65, 70, 75, 80, 85, 90, 91, 92, 93, 94, 95, 96, 97, 98, 99 or 100% identity to any one of SEQ ID NOs: 1-37 and SEQ ID NOs: 221-224. The amino acid sequences corresponding to SEQ ID NOs: 1-37 and SEQ ID NOs: 221-224 are shown in Table 1. As shown in the alignment of FIGS. 1A-1L, the family of nucleases described herein comprise regions of sequence similarity.

TABLE 1
Amino acid sequences of effectors of SEQ ID NOs: 1-37 and SEQ ID NOs: 221-224.

| SEQ ID | |
|---|---|
| NO | Amino Acid Sequence |
| 1 | MIKAYKFGLL NPISGEDQAA MDVLYLRNKL WNQLVELEKN SRAAYRALML DSSEELSVIQ |
| | TRIDAIEVER ADLVSQKKKL RASVRSKKVD TAGIDAAVER LIAERINLRA KAKQLREVVK |
| | VEIKPKAVEL DKVRYAAVLA LIKGSGLWWG NSETVIAAYD VARVRAMKES AELRFRSFDG |
| | TGKFAYRESG GIDFDKFMSG KVNFARLNTL PDSDFAHLSE RGRRSKARHH LTMTVLTSVD |
| | DAGKKVRHEV TWPIVMHRDM PAGAIKTIHV HRKRVGDQFN WTCSITIDVP EEPKQLIDHP |
| | AKAACGIDLG FRLVKDGLRI ATIADSDNRI EHVVLPLDWI EKMDYVEHLQ STLSETANLT |
| | WVRLRKHLSE LPDYPESIKE RIHNILKAGE RVPTRGMRSL LGALKAEPEL LPEALQILAA |
| | WSDDIYRPAR EMHNLRDKLM KRRQDLYRNV SHCLSNKYAM VRVEDMDLRQ IARVKKDDGS |
| | DNPLPDTVRD NRKRAALFEF VLSIKQSCVK TGSVFEKMNP AYSSMTCSSC GHLNQPGMDI |
| | HYSCENCGTL HDQDENAAKN FLRGEYFSSP KQDVA |
| 2 | MAVEERTKKG VSVRVYKYGL VPKGCLPDEA KDELLRANNL WNKLVEISRK NQSDFDEVRK |
| | KAHPPYGEEM VRLETINEKI DDAYDRKRDA RKEAGTRDET HPLIIKANAV IEILKEERSE |
| | IYDTLKLLRT EADKSVDKKA LNESFKSNIK LARRETSLNS DTKEEIIRND FRTARDRTFK |
| | TGGRLRFHTF DGTGYWHFRF RRNDEKGKKV DDYTIDDLFA GEKPSPKKPL PNRFKFLSRD |
| | DTRRKPRLRL RTTLAGGRIN ASKVFQEFDV IYHRPVPEGA IIPNAKILRT RTGDKFRYDL |
| | VLIVYLPEPK HKDIPLDDAI GIDLGFREDP DDKYRKQVGA IISLDPSDEV EEIFAPPKMV |
| | KAFGHIDELK SVLDESAADL GMKIKPLMKD IRLPEDNEKY KQEYKLWNSI VNARANVILS |
| | FEKAYKLALW CKKKDAGIPE DITGPVVYWW KGYSRRYREL HNLRKKQLLN RKDFYRQIAS |
| | RLVLRGLLIG VENFNLSKIA RSKDEDNVLN NKARANRELL SPSEFRAAIK NAADREGIPC |
| | LEVNPANTSK ICFDCGTLNK NLGSEKNWVC PACGCVHDRD TNAARNIAKR ALEKYLEDRK |
| | SAGK |
| 3 | MRTRVYKYGL VPIGYPPQEA VDELWRANNL WNTLVALHRE SRENWDDARR AASIAYSEKM |
| | DALEKKQAEI SQAFDALQQV RMEEGTKDES NPKLKLQRDV INRLKKEQGV IFDELKPLRK |
| | DADKVLDTKQ LNDDERKKVN TAASVKNNGL YNPTADEVVR NFKEARQKVL SNPAKSKLNI |
| | HSFDGTGYEN FRFRRKGEKQ DGVWFSELFA GNSKEDRRFA ILSKDESRKK IRLRLRVIMA |
| | GGATKASKVY QEFDWIYHRP IPEDAQIQNG KILRTRAGDK CRYDLVLILR LPDIDKIAPN |
| | DLDGTIGIDI GFRKSGDTVL IATVMSDEVS SKPQEIKAPT EMVSALEHVI NLQSELDDAA |
| | TDLGRTITPL LKANPPPQDH QKYRLWKSIA QRPSNETLSY EKAYKFAIWL NNEPDTFPKE |
| | ITEKVHTWWR SYSRKYREIH NRRRKQLTHR KHFYREAAAS IVAQRKLIVL EKIPLNKFAE |
| | TKDKDTKLNN RARAQRFLAS LSEFREAIKN AADREGVPCI EVNPAYTSKT CSDCGSLNKA |
| | LKAEKKWTCP SCGVVHDRDT NAANNLQKMG QIYIDDVKKT INEVLE |
| 4 | MAEAKAFTTR VYKYGAIPLG LFPKEGVDQL FRANQLWNKL VEINNEHAEY YDQARRDVDE |
| | EYNTLSVELD NVEDRVDKAF TGKRNARMKA SSRDADHPLV KTANDKIDTL FAERRELWDG |
| | IKQPRSRADK VIDKDVLNTA FNTAVKEAKR SDNTDGLDSN TANEVWRYFK EARGKVFQNP |
| | RSKLRFHRFD GMGYRFYRFR DRLIKTNKDG VSFEYFSRRG GDDDRAFLLT PSAKEPSTRD |
| | KRRGVQRYHL KLKVAGGHSK ASKVYAEFDL LMHRHIPDGA QINNAKLMRI RTGDRFKYTV |
| | NFSVRVPVAE PAKPKAHAIG VDIGFRRLED GSIRAATIAG TSSDFETLSV GLDQEYLDRI |
| | EHIESLQTKM DERATELGKK IKPLLKAGAV LKEGHDFYKF VKAIAVPRRK DVTMSLEQAY |
| | KLGSWLKNNP TELPKAVVEP VFDWWDHNAK HYREMHNLRR KTLAWRKEEY RFLAHGLVGH |
| | GLPIGIEAID LSTFAEVKDK DNKLRNKALS QRFLVSNSEL IGAIKNAAQR EGVPVIEVPA |
| | PYTSKTCSAC GNVHKELKAE LEWDCPECGV VHDRDENAAV NIARSAVKKM APKKPKALAA |
| | E |
| 5 | MAVEERTKKG VSVRVYKYGL VPKGCLPDEA KDELLRANNL WNKLVEISRK NQSDFDEVRK |
| | KAHPPYGEEM VRLETINKKI DDAYDRKRDA RKEAGTRDET HPLIIKANAV IEILKEERSE |
| | IYDTLKLLRT EADKSVDKKA LNESFKSNIK LARRETSLNS DTKEEIIRND FRTARDRTFK |
| | TGGRLRFHTF DGTGYWHFRF RRNDEKGKKV DDYTIDDLFA GEKPSPKKPL PNRFKFLSRD |
| | DTRRKPRLRL RTTLAGGRIN ASKVFQEFDV IYHRPVPEGA IIPNAKILRT RTGDKFRYDL |
| | VLIVYLPEPK HKDIPLDDAI GIDLGFREDP DDKYRKQVGA IISLDPSDEV EEIFAPPKMV |
| | KAFGHIDELK SVLDESAADL GMKIKPLMKD IRLPEDNEKY KQEYKLWNSI VNARANVTLS |
| | FEKAYKLALW CKKKDAGIPE DITGPVVYWW NKARANRELL SPSEFRAAIK NAADREGIPC |
| | RLVLRGLLIG VENFNLSKIA RSKDEDNVLN KGYSRRYREL HNLRKKQLLN RKDFYRQIAS |
| | LEVNPANTSK ICFDCGILNK NLGSEKNWVC PACGCVHDRD TNAARNIAKR ALEKYLEDRK |
| | SAGK |
| 6 | MTTRVYKYGL IPIGYPPQAA IDELFRANNL WNILVALHRE SRENWDDARR SASILYSEKM |
| | DELDKKNEDI SEAFNGLNQA RMDEGTKDET GNKRLQAERA IINRLKKEQK EIYAELKPLR |
| | KEADKTVDRK ALNDEYRNKC NTAVSAKVSG VYSRTAGELY AYFRTARDKA FKENATLRFH |
| | RFDGTGYFAF RCRSKTVGVN VDGISVEDFM SKQFIDYLRC AVQSVDESRK KPRIRINAVL |
| | TGGRTIASKV TQEFDWIYHR PLPPEGQIQN GKILRTRVGD KFKYDLVLTV KLPDVEMIKP |
| | AALNGTIGID VGFRKVGNSL LIGTVMSSDS AQKAVALEVP AMMVSALEHV VALQSELDDA |
| | ASDLGKAITP LLKANPIDEE HGKYRLWRSL ALRPLHVTLS FEQAYKLALW LKREPNLFPS |
| | EINEKVHTWW RSYSRKYRES HNRRKKQLTH RKHFYRETAA KLVAQNRLIV LEKINLTDFA |
| | ETKNKNTKLS NKARAQRFMA SLGEFRDAIK NAADREGVPV IDVNPAYTSK TCSECGYLNK |
| | ELKSEKEWNC PECGVVHDRD ENAANNLQKM GQKYLLDAAK TAPVVVK |
| 7 | MVSRQTDKDK TEVRVYKYGL IPVGYPPEET ISELWRANNL WNTLVGLHYK HREKYEEARC |
| | EAHLLYGEVA ERLAAIDEKI EQAYDDKRTA RMKAGIRDAA DPLIKEANAV IKALKDKRKT |
| | IYEELKPIRI EADKRVDRSV LNSAFTKDIN EATQSKNNGL YNTTANEVKR YFETARDRSF |
| | KTNSKLRFHR FDGTGFYAFR FRRKDNKVDG VSFEELFQGN KETDQRFTFI GRDESRKKTR |
| | LRLRATLAGG ARKASKIKHE FDLIYHRPIP DGSKIQNGKI MRTRIGDRFK YHLVLTIKQP |
| | KSEPLIVPKD VALGIDIGFR RIKDSIQVAS ITSSDPSLPS QNVMAPNKMI AGMERVIELQ |
| | SILDDAASEL GKLTKPILND NPQLEDHKRF GLWKAMARYP NNVTLSFETA YKVARWLQSE |
| | ADFIPPKAAK LVLEWWSAYS RRYRELHNLR AKQLLHRKHF YRQVASDLVA HRQLIVLEEI |
| | NLSVFAEVKD RDNNLSDKAR AQRFLAAPSE FRDAICNAAQ REKVPFVTVP PQYTSKMCSS |
| | CSVINRELDS QKKWECPSCG VTHDRDENAA RNIAKLGQKY FSGEKKKKK |
| 8 | MINCYKFGCL QPTAGFDQSA IEHLFLRNKL WNTLVALDHE FRQRYRDLML NSDEKLKSVQ |
| | DSIDSINQEI EDLVENKMKL RQKERTKNID SKLLDERINV LKAKRKTLSA DSKTERERVK |
| | VEIKPQIDLL NTERYEAKKL AYKESGLWWG NYETVVAAYD TASQKAMKSN TELRFKSFDG |
| | SGKFAVRFED GGLTIDELKA GASNLCRIET LNTSAFQNLS QRSIKSKARH SLIMTIYTEN |
| | DEKGKKQRKE ITVPIIFHRE MEEGKIKTIH LQRKRLGNQF TWSASFTLKN DIEPANVADH |
| | PATASCGIDL GYRLVKDGLR VATVADSQNN VEYLVLPKSW IDRMDYTETL QSGLSEAMTL |
| | MWAKLKAEIA KIPEYPDAVA EIIKNMQKMG DRLPYKGIKR LYRVLKEQDS TGSPVAGFNA |
| | VLDILKAWDK ATYRQELEMV NLKDKLLKQR EHIYRNFAAG LTKKYAHIVV EDMGLAELAK |
| | TEKSETETND MPNAVKANRQ RASLYSLVEA IRLSAAKVGS YFEKSKAAYS SMTCNVCGHL |
| | NPKTQNIHQS CESCNTMYDV DENAARNFLK GEYINEKVLK QG |
| 9 | MKRVTETDFT TRVYQYGVVP IDTFPEEGVE ELFRANKLWN DLVAFNRDHR EAYDQARSDA |
| | DEEYASLLQS LEKLNEKVDK AYDNKRNARL KAGTKDATHP LIKEANAKIR DLQIERRELW |
| | EPLKTARKSA DKKLDKKSFN DSFRITTNEL QQVKNTDGLQ NSTANLVADY FRTAKDRTFK |
| | DPKAKLQFHR FDGTGFFFYR FRKTGSNVDG VNFDELFARD EKDARPFVFL STDKSRSKPR |
| | LRLRIKVAGG QSVASKTYMH FDLILHRPVP MNAQVQSGKV VRKRVGDKES HTVSLILRIP |
| | ENGTVRLKKK AIGIDIGFRQ SGKIIRAAAI ASSDPKDPVE YIDVSEKFLK RIEYIDSLKS |
| | RLDEKATRLG EIIKPLLKKG KVLPEDHKKY RFVKSIANTP ANVILSFEKA YKLGSWMVKY |
| | GKGELPIEVE QEGIKWWEKN SLTYREHHNL RKKAYLERKD QYRNIASNLI KKKQPIGIEQ |
| | INLSVFAETK DKDNKLGNVA RLNRFLVAPS ELLNAIKNAA QREGVPVFEV PAKNTSKTCS |
| | SCGHIHKELG AEPNWTCPSC KAEHDRDHNA AINIARRAEE ELKKIDEKTK |
| 10 | MATRVYKYGL IPIGYPPKET IDELFKANVL WNNLVALHRK NREDWDDARR AASILYSDKI |
| | DELEKKEEDL DAAWKAFQQA RMDEGTRDET NNKRLKSERA SINRLKAERA EIYKELKPLR |
| | KEADKEIDKK QLNDSFRAQV NEALSVNNSG VYRAIADQIY ENFKTAKDKS IKENATLRFH |
| | RFDGTGYYHF RCRRKGTNVD GISIDDFMSR NFEAYPRCAV QNIDNSKKKP RIRINAVLAG |
| | GKSKASKIHQ EFDLIYHRPL PIDAQIQNGK ILRTRVGDKF KYDLVLILKI PDKEPISYNN |
| | LKGTIGIDIG FRRSVNSLLI GTVMSSDVTE EAYEIIVPPK IVEAFEHVID LQSELDDAAT |
| | DLGRIITPLL KAHPLDEDHS KYKMWRSLAL RPAHVTLSFE QAYKLAIWLK HEPDTFPEEI |
| | TKKVHTWWRS YSRKYRELHN RRRNQLTHRK HFYREEAAKI VALNKLIVLE EINLTDFAET |
| | KEKNTKLSKK ARAQRFMASL SEFRDAIKNA AQRDGIGIID VNPAYTSKTC SECGNLNKDL |
| | RSEKQWSCPA CGVVHDRDEN AANNLQKMGQ SYLENIKKET SEIIE |
| 11 | MATRAYKYGL IPIGYPPKET IDELFKANVL WNNLVALHRK NREDWDDARR AASVLYSDKI |
| | DELEKKEEDL DAAWKAFQQA RMDEGTRDET NNKRLKSERA SINRLKSERA EIYKELKPLR |
| | KEADKEIDKK QLNDSYRAQV NEALSVRNSG IYNATAGQIY ENFKTAKDKS IKENATLRFH |
| | RFDGTGYYHF RCRRKGAKVD GISVDDFMSR NFIANPRCAV QSIDNSKKKP RIRIDAVLVG |
| | GITKASKIHQ EFDLIYHRPL PIDAQIQNGK ILRTRVGDKF KYDLVLILKI PDKELISYNN |
| | LKGSIGIDIG FRRSVNSLLI GTVMSSDVTE KAYEIIVPPK IVEAFEHVID LQSELDDAAT |
| | DLGRIITPLL KAHPLDEDHS KYKMWRSLAL KPAHVILSFE QAYKLAIWLK HEPDTFPEEI |
| | TKKVHTWWRS YSRKYRELHN RRRNQLTHRK HFYREEAAKI VARNKLIVLE EINLTDFAET |
| | KEKNTKLSKK ARAQRFMASL SEFRDAIKNA AQRDGIGFID VNPAYTSKTC SECGNLNKGL |
| | RSEKQWSCPA CGVVHDRDEN AANNLQKMGQ TYLESVKKET SEVIE |
| 12 | MKRQAENVRS MVYQYGTVPA RVAPVEGEEL ALSQMRLAQR LWNVLVTIER ARVAGYRSIM |
| | RDEVQEQIDA LRERKDATWQ EIKATRQKAR AKVATPGLDA EMMRIKTALR LLVEHKQSTK |
| | QQRHDARREQ LNALAERANQ RIKRARQAAA SMGLFWGTYN AVIQSADAGR KHAGELRYQG |
| | FRGEGTVTAQ VMGGATPEQC VAGGHPFFQV APATPGQKWR YARVRIGSTS ERQPLWVAIP |
| | VVYHREIPAE ARIKSVSATR RILAGKVRWS LNVTVTLPPA EPRPAGQMVA IDIGWRLLPD |
| | GVRVAYWQDG TGNHSEVRIA DSDIAQFRKI SDLRSICDRA REEFLPSLVE WLKPYELDEE |
| | WTHRARALAQ WRSNDRIAAL IRWWADHRLS GDAEIYQTAV EWRRQYLHLA NWWRNQQEQM |
| | TLRVREQYRR FAAGIASQFA TVIVEDFDLR QVTETTEKAV GTYRQMVSPS LFRAAVINAC |
| | KREGVEIRIV SGAYSTGACH NCQHIEVWDQ AASILHRCGA CGALWDQDHN AAINLLASGG |
| | VVLWRINLVA AIGPLSQDRS QTGGKEAVES |
| 13 | MVSRQRKDSN KRILVYQYGA VPIGSFPEEG LNELWRINQL WNQLVELHNK NRQSYESARR |
| | AASDAYALVS ESIALKQAEI DQAFKDKRSA RMQAGTKDAD HPLIATANAV IDILMAERKT |
| | LYDQAKPLRK QADEGLDKKA LADQFQEAVK TARRVQQSGI SSVLADQVVA YFQTAREKAF |
| | KDAATLRFHR FDGSGYFFYR FRRPGSTSDG VSFKELFSDD PNNSEAFVFL SRDDSRPKKP |
| | RLRLRVKVAG GQTKASKRYA NFDLILHRPL PKDAQIQNAK LNRVRTGDKF DYRVSFTVKE |
| | EMPQAPQLQA GAIGVDIGFR QTTSGLLRAA ALAFVDAKAQ PLGEVRFIDV DPALLSRVGD |
| | QGHVNALKAQ LDDAAAALGR DIVPLLKAGA VLPEDHPKYK MVKAAANLPP NVTLSYERAY |
| | KLSTWSHFEP DALPLAVVNA LAHWRITYKH RYRELHNLRA KALLQRKHSY RQIAAELVRY |
| | RLPIGIELID LSKFAETKDA DNKLGNTARA NRFTVSPSEL LNAIKNAGER EGVPVYEVPP |
| | RDTSKRCHHC GHVHRALRAE QMWTCPNCAT QHDRDHNAAI NIARLALEKN STLTSESA |
| 14 | MAVEERTKKG VSVRVYKYGL VPKGCLPDEA KDELLRANNL WNKLVEISRK NQSDFDEVRK |
| | KAHPPYGEEM VRLETINEKI DDAYDRKRDA RKEAGTRDET HPLIIKANAV IEILKEERSE |
| | IYDTLKLLRT EADKSVDKKA LNESFKSNIK LARRETSLNS DTKEEIIRND FRTARDRTFK |
| | TGGRLRFHTF DGTGYWHFRF RRNDEKGKKV DDYTIDDLFA GEKPSPKKPL PNRFKFLSRD |
| | DTRRKPRLRL RTTLAGGRIN ASKVFQEFDV IYHRPVPEGA IIPNAKILRT RTGDKFRYDL |
| | VLIVYLPEPK HKDIPLDDAI GIDLGFREDP DDKYRKQVGA IISLDPSDEV EEIFAPPKMV |
| | KAFGHIDELK SVLDVSAADL GMKIKPLMKD IRLPEDNEKY KQEYKLWNSI VNARANVILS |
| | FEKAYKLALW CKKKDAGIPE DITGPVVYWW KGYSRRYREL HNLRKKQLLN RKDFYRQIAS |
| | RLVLRGLLIG VENFNLSKIA RSKDEDNVLN NKARANRELL SPSEFRAAIK NAADREGIPC |
| | LEVNPANTSK ICFDCGILNK NLGSEKNWVC PACGCVHDRD TNAARNIAKR ALEKYLEDRK |
| | SAGK |
| 15 | MTTRVYKYGL IPIGYPPQVA IDELFRANNL WNILVALHRE SRENWDDARR SASILYSEKM |
| | DELDKKNKDI REAFNGLNQA RMDEGTKDET GNKRLQAERA IINRLIKEQK EIYAELNPLR |
| | KEADKTVDKK ALNDEYRKKC NTAVSAKVSG VYSRTAGELY AYFRTARDKA FKDKTTLRFH |
| | RFDGTGYFAF RCRSKAVGVN VDGISVEDFM SQGEMDYMRC AVMSIDESKK KPRIRISAVL |
| | TGGATKASKV VQEFDWIYHR PLPPEGQIQN GKILRTRVGD KFKYDLVLTV KLPDVEMIQP |
| | AALNGTIGID VGFRKVGNSL LIGTVMFSDS AQKAVALEVP TMVVSALEHV DALRSELDDV |
| | ASDLGKAITP LLKANPIDEE HDKYRLWRSL ALRPLHVILS FEQAYKLALW LKREPNLFPS |
| | EINEKVHIWW RSYSRKYREI HNRRKKQLTH RKHFYRETAA KLIAQNKLIV LEKIDLTDFA |
| | ETKNKNTKLS NKARSQRFMA ALGEFRDAIK NAADREGVPV IDVNAAYTSK TCSECGYLNK |
| | ELKSEKEWNC PECGVVHDRD ENAANNLQKM GQKYLLDAAK TAVVVVK |
| 16 | MAPARQRGTE ALSTFVYQYG AIPKGPFPEQ GIESLYKENK LWNSLVEIHN RHRESYEAAR |
| | CAADSEYALI SESIKRAEAA VEAAFEAKRE ARKQAGTRDA SHPLIRAAID EINRLKQNRR |
| | ELWATAKAAR KRADGSIDKA ALNKAFRDAV NAAQRVGNTG GLNSTTANQV ADNFRTARER |
| | AFKEGARLRF HAFDGTGYWF FRFRVKGSKV DGISYGELFS QRVDDGRSFV LISAESDRKK |
| | PRIPLRVKVA GGAKEGSKVY AFFDLILHRP LPENAQVQNA KLIRKRNGDK FSYLVSLTVR |
| | VPRSMATEVQ AAAIGVDIGF RQLADQRVRV AAIGGSEPED ECQIVEVSKE FIRRLEHVDA |
| | LKASLDQSAE ALGQFIKPLL KAGSVLPEDH PRYRFVRSIA AAPPIVTMSF EKAYKLARWL |
| | LREPGALPPD VEKRALDWWQ QNGRRYRELH NLRQKALAAR KEEYRKIAAK IVKFGRPIGV |
| | EMIDLRVFAE AKDRDNKLGN TARSNRFLVA PSELLAAIRN AATKAGIPFH EVSARNTSKT |
| | CSACGVVNEK LGAEDSWICA VCGVVHDRDK NAAVNIARRA KEKRAPAAEG DS |
| 17 | MTTRVYKYGL IPIGYPPQVA IDELFRANNL WNTLVALHRE SRENWDDARR SASILYSEKM |
| | DELDKKNKDI REAFNGLNQA RMDEGTKDET GNKRLQAERA IINRLIKEQK EIYAELNPLR |
| | KEADKTVDKK ALNDEYRKKC NTAVSAKVSG VYSRTAGELY AYFRTARDKA FKDKTTLRFH |
| | RFDGTGYFAF RCRSKAVGVN VDGISVEDFM SQGEMDYMRC AVMSIDESKK KPRILISAVL |
| | TGGATKASKV VQEFDWIYHR PLPPEGQIQN GKILRTRVGD KFKYDLVLTV KLPDVEMIQP |
| | AALNGTIGID VGFRKVGNSL LIGTVMFSDS AQKAVALEVP TMVVSALEHV DALRSELDDV |
| | ASDLGKAITP LLKANPIDEE HDKYRLWRSL ALRPLHVTLS FEQAYKLALW LKREPNLFPS |
| | EINEKVHTWW RSYSRKYREI HNRRKKQLTH RKHFYRETAA KLIAQNKLIV LEKIDLTDFA |
| | ETKNKNTKLS NKARSQRFMA ALGEFRDAIK NAADREGVPV IDVNAAYTSK TCSECGYLNK |
| | ELKSEKEWNC PECGVVHDRD ENAANNLQKM GQKYLLDAAK TAVVVVK |
| 18 | MISRVYKYGA VPLKKFPEVK FPREQFPEEG VEELRRANKL RNSLVWLHRK NNEKFEAARV |
| | AADAEYGEIA EKLDALEKTI SQALTAKRQA RAKAGTRDAK HPLVKAASET INELTKQRSD |
| | LWKALKPARI RADKRVDRKA LTKQFDDAVK VVQHVKETGG LSSHCANEIV RYFKESRSRA |
| | LNERATLRYR RFDGTGFWFY RFREPGVNKN GVDFDGLLIG NKTEARDNRN FVLTEKSRRG |
| | KRVIYKLRAK IAGGAKKDSK VYGHEDLILH RPIPENARIQ SAKILRHRTG DKFTYTVSFT |
| | LKLPDVEQQT VEGSVLGLDI GFREMERNNS YRIATLATND QSRRVETIDI ARENRRGFLA |
| | RMNHIDDLRS TMDENATELG KKLLPLLKTA KPLPDSHQQF IFTERLRKTR ANVILDFERS |
| | YKMARWFIRA PDEADFYGPE IVGMVLRWWE ENSFKYREMH NLRRKALAER KEVYRMEAAR |
| | LVGFGIPIAV EKLDMSKWAE RKDSDNELSN RALSSRELVA PSELIAAIEN AAKREGVPFI |
| | KVNAANTSKA CHACGTINKA LKGELIWTCE ECETKHDRDI NAAINIAKRG ILQAKKEKKQ |
| 19 | MIKAFKFGML APVSGFDQAA LDVLYLRNKL WNALVEQERK HRERYRALLT GSNDELSSIQ |
| | SRLDEIERER SDLVARKKKV RAKVRSKNVD TSEIDAAIDV LIAERDELRG KAKALRAEVK |
| | EKIKPLIANL DRERYETIKQ LTKESGLWWC NYETVVAAYD VARVKAMKAN AELRFRSFDG |
| | TGKFAVRQSG GFPLSDLTAG KLSFARLEAL PEDGFAHLSE RGKRSMARHH LIMTIMTYKD |
| | ESGKLRRHEV TWPIILHRPL PYGTVKFIYV QRKRVGKDFK WTCSITMEID EIEKTLIDHT |
| | SRAACGIDLG YRLVTDGLRV ATLADTEGKV RHLVLPQDWL DRMDHVERIQ GWLSDQNNLA |
| | WEKLKALLIA LQEYPEPLAE SIGRLLKAGD KTPVRGMRAL HWRLRNEPEI MPEALKVLDE |
| | WEVKTRRREQ EMYNLRDKLI NRRKDIYRNF AYQVANGYAL IRIEDIQLKK LAMVKFDDGR |
| | DNPLPQAVRN NRTRAALYEL ALYIQQAAAK TGAEFEKMPA MNSTLICSNC GHQNTGMDKR |
| | DIHFRCEKCD TLHDQDQNAA KNFLRGQEFY LAEQMVA |
| 20 | MATRVYKYGL IPIGYPPKET IDELFKANVL WNNLVALHRK NREDWDDARR AASILYSDKI |
| | DELEKKEEDL DAAWKAFQQA RMDEGTRDET NNKRLKSERA SINRLKAERA EIYKELKPLR |
| | KEADKEIDKK QLNDSYRAQV NEAISVRNSG IYNATAGQVL DNFKAARDRS FKENATLKFH |
| | RFDGTGYYHF RCRRRGAKVD GINVEDFMSR NFIANPRCAV QSIDNSKKKP RIRINAVLAG |
| | GQSKASKVHQ EFDLIYHRPL PIDAQIQNGK ILRTRVGDKF KYDLVLTLKI PDKEPISYNN |
| | LKGTIGIDIG FRRSVNSLLI GTVMSSNVSE KAYEIKVPPK IVEAFEHVID LKSELDDAAT |
| | DLGRIITPLM KAHPLDEDHS KYKMWRSLAL RPAHVILSFE QAYKLAIWLK HEPDTFPEEI |
| | TKKVHTWWRS YSRKYRELHN RRRNQLTHRK HFYREEAAKI VALNKLIVLE EINLTDFAET |
| | KEKNTKLSKK ARAQRFMASL SEFRDAIKNA AQRDGIGIID VNPAYTSKTC SECGNLNKDL |
| | RSEKQWSCPA CGVVHDRDEN AANNLQKMGQ TYLESLKKET SEVIE |
| 21 | MAVEERTKKG VSVRVYKYGL VPKGCLPDEA KDELLRANNL WNKLVEISRK NQSDFDEVRK |
| | KAHPPYGEEM VRLETINEKI DDAYDRKRDA RKEAGTRDET HPLIIKANAV IEILKEERSE |
| | IYDTLKLLRT EADKSVDKKA LNESFKSNIK LARRETSLNS DTKEEIIRND FRTARDRTFK |
| | TGGRLRFHTF DGTGYWHFRF RRNDEKGKKV DDYTIDDLFA GEKPSPKKPL PNRFKFLSRD |
| | DTRRKPRLRL RITLAGGRIN ASKVFQEFDV IYHRPVPEGA IIPNAKILRT RTGDKFRYDL |
| | VLTVYLPEPK HKDIPLDDAI GIDLGFREDP DDKYRKQVGA IISLDPSDEV EEIFAPPKMV |
| | KAFGHIDELK SVLDESAADL GMKIKPLMKD VRLPEDNEKY KQEYKLWNSI VNARANVILS |
| | FEKAYKLALW CKKKDAGIPE DITGPVVYWW KGYSRRYREL HNLRKKQLLN RKDFYRQIAS |
| | RLVLRGLLIG VENFNLSKIA RSKDEDNVLN NKARANRFLL SPSEFRAAIK NAADREGIPC |
| | LEVNPANTSK ICFDCGTLNK NLGSEKNWVC PACGCVHDRD TNAARNIAKR ALEKYLEDRK |
| | SAGK |
| 22 | MATRVYKYGL IPIGYPPKET IQELFRANVL WNNLVALHKK NREDWDDARR AASILYSDKI |
| | DELEKNKEDL DAVWKAFNQA RMDEGTRDET NNKRLKSERA SIDRLDAVRA EIYKELKPLR |
| | KEADKEIDKK QLNDSYRRKV NEAVSVRNSG IYNATAGQVL DNFKTARDRS FKENATLKFH |
| | KFDYTGYYHF RCRRKGAKVD GISVDDFMSR NFIANPRCAV QSIDNSKKKP RIRINAVLVG |
| | GQSKASKIHQ EFDLIYHRPL PIDAQIQNGK ILRTRVGDKF KYDLVLILKI PDKEPISYNN |
| | LKGTIGIDIG FRRSVNILLI GTVMSSDLTE KAYEIKVPPK IVEAFEHIID LQSELDDAAT |
| | DLGRIITPLL KAHPLDEDHS KYKMWRSLAL RPAHVTLSFE QAYKLSIWLK DEPDTFPEEI |
| | TKKVHTWWRS YSRKYRELHN RRRNQLTHRK HFYREEAAKI VALNKLIVLE EINLTDFAET |
| | KEKNTKLSKK ARAQRFMASL SEFRDAIKNA AQRDGIGIIN VNPAYTSKTC SECGNLNKDL |
| | RSEKQWSCPA CGVVHDRDEN AANNLQKMGQ TYLESLKKET SEVIE |
| 23 | MAQPHLFLLY TQAASSIIFD MTTRVYKYGL IPIGYPPKAA IDELFRANNL WNTLVALHRE |
| | SRENWDDARR SACILYSEKM DELDKKNEDI SEAFNGLNQA RMDEGTKDET GNKRLQAERA |
| | IINRLKKERN EIYAELKPLR KEADKTVDRK ALNDEYHKKC NRAVSAKVSG VYSGTAGELY |
| | AYFKTARGKA FKENATLRFH RFDGTGYFAF RCRSKTVGVN VDGISVEDFM SQGELDYMRC |
| | AVMSVDESKK KPRIRISAVL TGGATKASKV FQEFDWIYHR PLPPEGQIQN GKILRTRVGD |
| | KFKYDLVLTV KLPDVEMIKP AALNGTIGID VGFRKVGNSL LIGTVMSSDS AQKAVALEVP |
| | TMMVSALEHV DALRSELDDA ASDLGKAITP LLKANPIDEE HDKYRLWRSL ALRPLHVTLS |
| | FEQAYKLALW LKREPNLFPS EINEKVHTWW RSYSRKYRES HNRRKKQLSH RKHFYRETAA |
| | KLVAQNKLIV LEKIDLNVFA ETKNKNTKLS NKARAQRFMA SLGEFRDAIK NAADREGVPV |
| | IDVNPAYTSK TCSECGYLNK ELKSEKEWNC PDCGVVHDRD ENAANNLQKM GQKYLLDAAK |
| | TAAVVVK |
| 24 | MATRVYKYGL IPIGYPPKET IDELFKANVL WNNLVALHRK NREDWDDARR AASILYSDKI |
| | DELEKKEEDL DAAWKAFQQA RMDEGIRDET NNKRLKSGRA SINRLDAEKA EIYKELKPLR |
| | KEADKEIDKK QLNDAYRTKV NEAVSVRNSG IYSATAGQIL ENFKTARDRS FKESATTLRF |
| | HRFDGTGYYQ FRCRRKGINV DGISIDDEMS RNFEANPRCA VQSIDNSKKK PRIRIDAVLV |
| | GGQSKASKIH QEFDLIYHRP LPIDAQIQNG KILRTRVGDK FKYDLVLILK IPDKEPISYN |
| | NLKGTIGIDI GFRRSVNSLL IGTVMSSDVT EKAYEIKVPP KIVEAFVHVI DLQSELDDAA |
| | TDLGRIITPL LKAHPLDENH SKYKMWRSLA LRPAHVTLSF EQAYKLAIWL KHEPDTFPEE |
| | ITKQVHTWWR SYSRKYRELH NRRRNQLTHR KHFYREEAAK IVALNKLIVL EEINLTDFAE |
| | TKEKNTKLSK KARAQRFMAS LSEFRDAIRN AAQRDGIGII DVNPAYISKT CSECGNLNKG |
| | LRSEKQWSCP ACGVVHDRDE NAANNLQKMG QSYLESVKKE TSEVIE |
| 25 | MAESTGFNMT TRVYKYGLIP IGYPPQAAID ELFRANKLWN TLVALHRESR ENWDDARRSA |
| | SILYSEKMDE LDKKNDDVSE AFNGLNQARM EEGTKDETGN KRLLAERSII NMLKEDRAQI |
| | YAELNPLREK ADKEIDKKAL NDAYRKKCNE AVSAKVSGVY SSTAGQVYAN FRTARDKAFK |
| | DNTTTKFHRF DGTGYFAFRC RSKDTSVNVD GISVEDFMSQ GFVDYMRCAV MSVDESKKKP |
| | RIRINAVLIG GRTKASKVFQ EFDWIYHRPL PPESQIQNGK ILRTRVGDKF RYDLVLTVKL |
| | PDVEMVKPAA LNGTIGIDVG FRKSGNTLLI GTVMSSDSAQ KAVALEVPTM MVSALEHVVA |
| | LQSELDDAAS DLGKAITPLL KANPIDEEHS KYRLWRSLAL RPMHVTLSFE QAYKLSLWLK |
| | HEPDLFPSEI NEKVHTWWRS YSRKYREIHN RRKKQLTHRK HFYRETAAKL VAQNKLIVLE |
| | KIKLTDFAET KGKNTKLSNK ARAQRFMASL AEFRDAIKNA ADREGVPVID VNPAYTSKTC |
| | SDCGYRNKEL RSEKEWTCPE CGVDHDRDEN AANNLQKMGQ KYLLDIEKAV SVVVE |
| 26 | MATRVYKYGL IPIGYPPKET IQELFRANVL WNNLVALHKK NREDWDDARR AASILYSDKI |
| | DELEKNKEDL DAVWKAFNQA RMDEGTRDET NNKRLKSERA SIDRLDAVRA EIYKELKPLR |
| | KEADKEIDKK QLNDSYRRKV NEAVSVRNSG IYNATAGQVL DNFKTARDRS FKENATLKFH |
| | KFDYTGYYHF RCRRKGAKVD GISVDDEMSR NFIANPRCAV QSIDNSKKKP RIRINAVLVG |
| | GQSKASKIHQ EFDLIYHRPL PIDAQIQNGK ILRTRVGDKF KYDLVLILKI PDKEPISYNN |
| | LKGTIGIDIG FRRSVNILLI GTVMSSDLTE KAYEIKVPPK IVEAFEHIID LQSELDDAAT |
| | DLGRIITPLL KAHPLDEDHS KYKMWRSLAL RPAHVILSFE QAYKLSIWLK HEPDTFPEEI |
| | TKKVHTWWRS YSRKYRELHN RRRNQLTHRK HFYREEAAKI VALNKLIVLE EINLTDFAET |
| | KEKNTKLSKK ARAQRFMASL SEFRDAIKNA AQRDGIGIIN VNPAYTSKTC SECGNLNKDL |
| | RSEKQWSCPA CGVVHDRDEN AANNLQKMGQ TYLESLKKET SEVIE |
| 27 | MATRVYKYGL IPIGYPPKET IQELFRANVL WNNLVALHKK NREDWDDARR AASILYSDKI |
| | DELEKNKEDL DAVWKAFNQA RMDEGTRDET NNKRLKSERA SIDRLDAVRA EIYKELKPLR |
| | KEADKEIDKK QLNDSYRRKV NEAVSVRNSG IYNATAGQVL DNEKTARDRS FKENATLKFH |
| | KFDYTGYYHF RCRRKGAKVD GISVDDEMSR NFIANPRCAV QSIDNSKKKP RIRINAVLVG |
| | GQSKASKIHQ EFDLIYHRPL PIDAQIQNGK ILRTRVGDKF KYDLVLILKI PDKEPISYNN |
| | LKGTIGIDIG FRRSVNSLLI GTVMSSDVTE EAYEIIVPPK IVEAFEHVID LQSELDDAAT |
| | DLGRIITPLL KAHPLDEDHS KYKMWRSLAL RPAHVILSFE QAYKLAIWLK HEPDTFPEEI |
| | TKKVHTWWRS YSRKYRELHN RRRNQLTHRK HFYREEAAKI VALNKLIVLE EINLTDFAET |
| | KEKNTKLSKK ARAQRFMASL SEFRDAIKNA AQRDGIGIID VNPAYTSKTC SECGNLNKDL |
| | RSEKQWSCPA CGVVHDRDEN AANNLQKMGQ SYLENIKKET SEIIE |
| 28 | MATRVYKYGL IPIGYPPKET IDELFKANVL WNNLVALHRK NREDWDDARR AASILYSDKI |
| | DELEKKEEDL DAAWKAFQQA RMDEGTRDET NNKRLKSGRA SINRLDAEKA EIYKELKPLR |
| | KEADKEIDKK QLNDAYRTKV NEAVSVRNSG IYSATAGQIL ENFKTARDRS FKESATTLRF |
| | HRFDGTGYYQ FRCRRKGTNV DGISIDDEMS RNFEANPRCA VQSIDNSKKK PRIRIDAVLV |
| | GGQSKASKIH QEFDLIYHRP LPIDAQIQNG KILRTRVGDK FKYDLVLILK IPDKEPISYN |
| | NLKGTIGIDI GFRRSVNTLL IGTVMSSDVT EKAYEIKVPP KIVEAFVHVI DLQSELDDAA |
| | TDLGRIITPL LKAHPLDENH SKYKMWRSLA LRPAHVTLSF EQAYKLAIWL KHEPDTFPEE |
| | ITMKVHTWWR SYSRKYRELH NRRRNQLTHR KHFYREEAAK IVALNKLIVL EEINLTDFAE |
| | TKEKNTKLSK KARAQRFMAS LSEFRDAIRN AAQRDGIGII DVNPAYTSKT CSECGNLNKG |
| | LRSEKQWSCP ACGVVHDRDE NAANNLQKMG QSYLESVKKE TSEVIE |
| 29 | MTIKVYKFGL LDPVSGWDQT AIDVLFLRNK LWNNLVAMEH DKRQAYRNLL LDSDTELAAL |
| | QARLDAIEVE KASLITSKKA LRAKARSRQV DTAEIDLEIK KLLEERKALG GQTKDLRERV |
| | KIEVKPLAAE LDQQRYEKTK QLNKESGLWW CNSMTVIAAY EVGRLRAMRE KNELRFHGFD |
| | GTGKYSVCRT GGFSLDHVMT GKLSFVSIRT LPIANLDDLS ERGQRSRARH HLTMIVLRAT |
| | TEEGTKIRHE VTWPIILHRP LPDDCLIKQI QVLRKRVGDR FEWTCSITVD TPEELKARLD |
| | SPSISVCGID LGFRQVNNDL RVATLADSSG GLRYYTIGKD WLDSMDYVEA IQSDLSGTAN |
| | SVWAQLRLIL KELDEYPEAL RERITDMLKA GAKTPIRAMR AMQKILSNEP DLMPDALALL |
| | DDWKKRIRRR TKEMHDLRDK LINRRKDIYR NIACEIARDY SLVRIANLKL KDMVKLKRND |
| | GTDTKLIDNA RKNCNRAALS ELTLYIQQAC AKNGVALEKI DTTYMTRICY QCGYLNPANT |
| | INLLLSCEGC GAEYDQDDNA AKNYLNATKP GTG |
| 30 | MKALVSILGM ATRVYKYGLV PIGYPPEAVV GLYGELHKAN SLWNILVALH KESRENWDDA |
| | RRSASINYSE KMDELDAKQQ EISQAFDALR QVRMEEGTKD ESNPKLKLER DIINRLKKEQ |
| | GVIFAELKPL RKAADDNVDK KALNEAYRDK CKQAVSVKNC GIYRRTADQI YANFRTAREK |
| | AFKENATMRF HPFDGTGYFQ FRCTRKGSNT DGITVDEFMK ASFTENNRCA VQTVDDSKKK |
| | PRIRINSVLT GGPSRATKVF QEFDWIYHRP LPPDAQIQNG KILRTRVGDK FRYDLVLTIK |
| | VPDTELIKAD KLNGTIGIDV GFRKSGDTIL IGTVMSEDAS QKAQEIKAPS KMVSALEHII |
| | ELQSELDDAA TDLGKALTPL LLANPLPDDH PKFRMWQSLA RRPAHVILSF EKAYKFALWL |
| | NREPNVFPKE VTEKVHTWWR SYSRKYREIH NRRKKQLTHR KHFYRETAAS IVAQKKLIVL |
| | EDINLTDFAE TRDKNTKLSN KARAQRFLAS LSEFRDAIKN AAEREGVKVI DVDPAYTSKT |
| | CSDCSFLNKA LKSDKEWSCP NCGVVHDRDT NAANNLQKMG QVYIEEVKKE ALKVIE |
| 31 | MGSRGLVKLV EPARLNFPEK NKKGGNAMIS RVYKYGAVPL KKFPEVKFPR EQFPEEGVEE |
| | LRRANKLRNS LVWLHRKNNE KFEAARVAAD AEYGEIAEKL DALEKTISQA LTAKRQARAK |
| | AGTRDAKHPL VKAASETINE LTKQRSDLWK ALKPARIRAD KRVDRKALTK QFDDAVKVVQ |
| | HVKETGGLSS HCANEIVRYF KESRSRALNE RATLRYRRFD GTGFWFYRFR EPGVNKNGVD |
| | FDGLLIGNKT EARDNRNFVL TEKSRRGKRV IYKLRAKIAG GAKKDSKVYG HFDLILHRPI |
| | PENARIQSAK ILRHRTGDKF TYTVSFTLKL PDVEQQTVEG SVLGLDIGER EMERNNSYRI |
| | ATLATNDQSR RVETIDIARE NRRGFLARMN HIDDLRSTMD ENATELGKKL LPLLKTAKPL |
| | PDSHQQFIFT ERLRKTRANV ILDFERSYKM ARWFIRAPDE ADFYGPEIVG MVLRWWEENS |
| | FKYREMHNLR RKALAERKEV YRMEAARLVG FGIPIAVEKL DMSKWAERKD SDNELSNRAL |
| | SSRFLVAPSE LIAAIENAAK REGVPFIKVN AANTSKACHA CGTINKALKG ELIWTCEECE |
| | TKHDRDINAA INIAKRGILQ AKKEKKQ |
| 32 | MATRVYKYGL IPIGYPAKET IDELFKANVL WNNLVALHRK NREDWDDARR AASVLYSDKI |
| | DDLEKKEEDL DAAWKAFQQA RMDEGTRDET NNKRLKSERA SINRLDTEKA EIYKELKPLR |
| | KEADKEIDKK QLNDAYRTKV NEAVSVRNSG IYSATAGQIL ENFKTARDRS FKESATTLRF |
| | HRFDGTGYYQ FRCRRKGTNV DGISIDDEMS RNFEANPRCA VQSIDNSKKK PRIRIDAVLV |
| | GGQSKASKIH QEFDLIYHRP LPIDAQIQNG KILRTRVGDK FKYDLVLILK IPDKEPISYN |
| | NLKGTIGIDI GFRRSVNSLL IGTVMSSDVT EEAYEIIVPP KIVEAFEHVI DLKSELDDAA |
| | TDLGRIITPL MKAHPLDEDH SKYKMWRSLA LRPAHVILSF EQAYKLAIWL KHEPDTFPEE |
| | ITKKVHTWWR SYSRKYRELH NRRRNQLTHR KHFYREEAAK IVALNKLIVL EEINLTDFAE |
| | TKEKNTKLSK KARAQRFMAS LSEFRDAIKN AAQRDGIGII DVNPAYISKT CSECGNLNKD |
| | LRSEKQWSCP ACGVVHDRDE NAANNLQKMG QTYLESLKKE TSEVIE |
| 33 | MAESTGFNMT TRVYKYGLIP IGYPPQAAID ELFRANKLWN TLVALHRESR ENWDDARRSA |
| | SILYSEKMDE LDKKNDDVSE AFNGLNQARM EEGTKDETGN KRLLAERSII NMLKEDRAQI |
| | YAELNPLREK ADKEIDKKAL NDAYRKKCNE AVSAKVSGVY SSTAGQVYAN FRTARDKAFK |
| | DNTTTKFHRF DGTGYFAFRC RSKDTSVNVD GISVEDFMSQ GFVDYMRCAV MSVDESKKKP |
| | RIRINAVLTG GRTKASKVFQ EFDWIYHRPL PPESQIQNGK ILRTRVGDKF RYDLVLTVKL |
| | PDVEMVKPAA LNGTIGIDVG FRKSGNTLLI GTVMSSDSAQ KAVALEVPKM MVSALEHVVA |
| | LQSELDDAAS DLGKAITPLL KANPIDEEHS KYRLWRSLAL RPMHVTLSFE QAYKLSLWLK |
| | HEPDLFPSEI NEKVHTWWRS YSRKYREIHN RRKKQLTHRK HFYRETAAKL VAQNKLIVLE |
| | KIKLTDFAET KGKNTKLSNK ARAQRFMASL AEFRDAIKNA ADREGVPVID VNPAYTSKTC |
| | SDCGYRNKEL RSEKEWTCPE CGVDHDRDEN AANNLQKMGQ KYLLDIEKAV SVVVE |
| 34 | MTTRVYKYGL IPIGYPPQAA IDELFRANNL WNILVALHRE SRENWDDARR SASILYSEKM |
| | DELDKKNEDI SEAFNGLNQA RMDEGTKDET GNKRLQAERA IINRLKKERN EIYAELKPLR |
| | KEADKTVDRK ALNDEYRKKC NTAVSAKVSG VYSGTAGELY AYFRTAKDKA FKENATLRFH |
| | RFDGTGYFAF RCRSKAVGVK VDGISVEDFM SKQFIDYLRC AVQSVDESRK KPRIRINAVL |
| | TGGRTTASKV FQEFDWIYHR PLPPEGQIQN GKILRTRVGD KFKHDLVLIV KLPDVEMIKP |
| | AALNGTIGID VGFRKVGNSL LIGTVMYSDS AQKAEALEVP TMMVSALEHV VALQSELDNA |
| | ASDLGKAITP LLKANPIDEE HDKYRLWRSL ALRPLHVTLS FEQAYKLALW LKREPNLFPS |
| | EINEKVHTWW RSYSRKYREI HNRRKKQLTH RKHFYRETAA KLVAQNKLIV LEKINLTDFA |
| | ETKNKNTKLS NKARAQRFMA ALGEFRDAIK NAADREGVPV IDVNPAYTSK TCSECGYLNK |
| | ELKSEKEWNC PDCGVVHDRD ENAANNLQKM GQKYLLDAAK TAAVVVK |
| 35 | MAVEERTKKG VSVRVYKYGL VPKGCLPDEA KDELLRANNL WNKLVEISRK NQSDFDEVRK |
| | KAHPPYGEEM VRLETINEKI DDAYDRKRDA RKEAGTRDET HPLIIKANAV IEILKEERSE |
| | IYDTLKLLRT EADKSVDKKA LNESFKSNIK LARRETSLNS DTKEEIIRND FRTARDRTFK |
| | TGGRLRFHTF DGTGYWHFRF RRNDEKGKKV DDYTIDDLFA GEKPSPKKPL PNRFKFLSRD |
| | DTRRKPRLRL RTTLAGGRIN ASKVFQEFDV IYHRPVPEGA IIPNAKILRT RTGDKFRYDL |
| | VLIVYLPEPK HKDIPLDDAI GIDLGFREDP DDKYRKQVGA IISLDPSDEV EEIFAPPKMV |
| | KAFGHIDELK SVLDESAADL GMKIKPLMKD IRLPEDNEKY KQEYKLWNSI VNARANVILS |
| | FEKAYKLALW CKKKDAGIPE DITGPVVYWW KGYSRRYREL HNLRKKQLLN RKDFYRQIAS |
| | RLVLSGLLIG VENFNLSKIA RSKDEDNVLN NKARANRELL SPSEFRAAIK NAADREGIPC |
| | LEVNPANTSK ICFDCGILNK NLGSEKNWVC PACGCVHDRD TNAARNIAKR ALEKYLEDRK |
| | SAGK |
| 36 | MYHFSMATRV YKYGLVPLGY PPDSVVGKGG ELQRANNLWN TLVALHRESR EHWDDARRSA |
| | SILYSEKMDE LDKKNEDIND AFDGLNKARM EEGTKDETGN KRLLAERAII NRLKKEQGDI |
| | YVELKPLRKE ADKTVDRKAL NDEYRNKCNT AVSAKVSGVY SRTAGELYAY FRTARDKAFK |
| | DNATLRFHRF DGTGYFAFRC RSKAVGVKVD GISVEDFMSQ GFVDYMRCAV MSVDESKKKP |
| | RIRISAVLTG GATKASKVFQ EFDWIYHRPL PPEGQIQNGK ILRTRVGDKF KYDLVLTVKL |
| | PDVEMIEPAA LNGTIGIDVG FRKVGNSLLI GTVMSSDSAQ KAVVLEVPTM VVSALEHVDA |
| | LRSELDDAAS DLGKAITPLL KANPIDEEHD KYRLWRSLAL RPLHVILSFE QAYKLALWLK |
| | REPNLFPSEI NEKVHTWWQS YSRKYRESHN RRKKQLSHRK HFYRETAAKL VAQNKLIVLE |
| | KIDLTVFAET KNKNTKLSNK ARSQRYMAAL GEFRDAIKNA ADREGVPVID VNPAYTSKTC |
| | SECGYLNKEL KSEKEWNCPE CGVVHDRDEN AANNLQKMGQ KYLSSRCKSS YGSS |
| 37 | MVSRQTDKDK TEVRVYKYGL IPVGYPPEET ISELWRANNL WNTLVGLHYK HREKYEEARC |
| | EAHLLYGEVA ERLAAIDEKI EQAYDDKRTA RMKAGTRDAA DPLIKEANAV IKALKDKRKA |
| | IYEELKPIRI EADKRVDKSA LNSAFTKDIN EATQSKNIGG LYSTTANEVK ESFKTARDRS |
| | FKTNSKLRFH RFDGTGFYAF RFRRKDNKVD GVSFEELFQG NKETDQRFTF IGRDESRKKT |
| | RLRLRATLAG GARKASKIKH EFDLIYHRPI PDGSKIQNGK IMRTRIGDRF KYHLVLTIKQ |
| | PKSEPLIVPK DVALGIDIGF RRIKDSIQVA SITSSDPSLP SQNVMAPNKM IAGMERVIEL |
| | QSILDDAASE LGKLIKPILN DNPQLEDHKR FGLWKAMARY PNNVTLSFET AYKVARWLQS |
| | EADFIPPKAA KLVLEWWSAY SRRYRELHNL RAKQLLHRKH FYRQVASDLV AHRQLIVLEE |
| | INLSVFAEVK DRDNNLSDKA RAQRFLAAPS EFRDAICNAA QREKVPFVTV PPQYTSKMCS |
| | SCSVINRELD SQKKWECPSC GVTHDRDENA ARNIAKLGQK YFSGEKKKKK |
| 221 | MATRVYKYGL IPIGYPAKET IDELFKANVL WNNLVALHRK NREDWDDARR AASVLYSDKI |
| | DDLEKKEEDL DAAWKAFQQA RMDEGTRDET NNKRLKSERA SINRLDTEKA EIYKELKPLR |
| | KEADKEIDKK QLNDAYRTKV NEAVSVRNSG IYSATAGQIL ENFKTARDRS FKESATTLRF |
| | HRFDGTGYYQ FRCRRKGINV DGISIDDEMS RNFEANPRCA VQSIDNRKKK PRIRIDAVLV |
| | GGQSKASKIH QEFDLIYHRP LPIDAQIQNG KILRTRVGDK FKYDLVLILK IPDKEPISYN |
| | NLKGTVGIDI GFRRSVNSLL IGTVMSSDVT EKAYEIKVPP KIVEAFEHVI DLQSELDDAA |
| | TDLGRIITPL LKAHPLDEDH NKYKMWRSLA LRPAHVILSF EQAYKLAIWL KHETDTFPEE |
| | ITKKVHTWWR SYSRKYRELH NRRRNQLTHR KHFYREEAAK IVALNKLIVL EEINLTDFAE |
| | TKEKNTKLSK KARAQRFMAS LSEFRDAIRN AAQRDGIGII DVNPAYTSKT CSECGNLNKD |
| | LKSEKQWSCP ACGVVHDRDE NAANNLQKMG QTYLESLKKE TSEVIE |
| 222 | MATRVYKYGL IPIGYPPQAA IDELFRANSL KNTLVALHRE SRENWDDARR SASILYSEKM |
| | DELDKKNEDI TEAFNGLNKA RMDEGTKDET GNKRLLAERA IINRLKKEKG DIYAELKPLR |
| | KEADKSIDKK ALNDAYRQKC NDAVSAKVSG VYRRTAEQIY ANFKTAKDKA SKDNATLQFH |
| | RFDGTGYFQF RCNPKGVSTD GISVDAFMSA NEDGYMRCAV QSVDNSKKKP RIRINAVLAG |
| | GRTKASKVFQ EFDWIYHRPL PADAQIQNGK ILRTRVGDKF RYDLVLTIRV PDVEMVQPAK |
| | LSGTIGIDVG FRKVGNTLLI GTVMSSDRSQ KAVALEVPQM MVSALEHVVA LQGELDDAAS |
| | DLGKAITPLL KANPIDDEHS KYRLWRSLAL RPLHVILSFE QAYKLSLWLK HEPSLFPSEI |
| | NLKVHTWWRS YSRKYREIHN RRKKQLTHRK HFYRETAAKL VAENKLIVLE DINLTDFAET |
| | KSKNTKLSNK ARAQRFMASL GEFRDAIKNA AGREGVPVID VNPAYTSKTC SDCGHLNKEL |
| | RSEKEWTCPA CGVVHDRDEN AANNLQKMGQ KYLLDVQKAA SMVVQ |
| 223 | MIKAFKYGML EPVAGFDKAA IDVLYLRNKL WNSLVELEKA HRERYRILIT GSDDELSKIQ |
| | ARLDQIEAER AELVKRKRQA RAMVRSKKVD TSEHDDRIDM LMAERNDLRT KAKDIRLQVK |
| | EKVKPAIADL EKERYEAVKH LIHEAGLWWC NSETVIAAYD LARVKAMKEN AELRFRSFDG |
| | SGKFAVRKTG GFALSDLVSG KLSFARLEAL PDANFAHLSE RGKRSRARHH LTMTILTYKD |
| | ESGKLCRHEV TWPIILHRPL PPEGMIKFIH VQRKRIGKDF QWTCSITMEV DEIQKTPIDH |
| | PSRAACGIDI GYRLVKDGLR VAVIADISGK IDHLTLPQDW IEKMDHVESI QGHLDNSNDL |
| | AWGELKALLK SMHDYPESIA ESIGRLLKAG DRIPVRGMRA LHWRLRNEPE TMPEVLSILD |
| | TWEAETCRRE REMHRLRRKL INRRKDLYRN FAYKVANRYV LIRIRGLSLK KLAAVNLEDG |
| | SDNQMPQAVR NNRTRASLSE LILCLQQAAV KAGADFEKVF DVNSTTTCST CGNQNLKMDR |
| | EDIYFRCEKC DTLHDQDENA AKNLLRKEFY LAEQAVM |
| 224 | MINCYKFGCL QPTAGFDQSA IEHLFLRNKL WNTLVALDHE FRQRYRDLML NSDEKLKSVQ |
| | DSIDSINQEI EDLVENKMKL RQKERTKNID SKLLDERINV LKAKRKTLSA DSKTERERVK |
| | VEIKPQIDLL NTERYEAKKL AYKESGLWWG NYETVVAAYD TASQKAMKSN TELRFKSFDG |
| | SGKFAVRFED GGLTIDELKA GASNLCRIET LNTSAFQNLS QRSIKSKARH SLIMTIYTEN |
| | DEKGKKQRKE ITVPIIFHRE MEEGKIKTIH LQRKRLGNQF TWSASFTLKN DIEPANVADH |
| | PATASCGIDL GYRLVKDGLR VATVADSQNN VEYLVLPKSW IDRMDYTETL QSGLSEAMTL |
| | MWAKLKAEIA KIPEYPDAVA EIIKNMQKMG DRLPYKGIKR LYRVLKEQDA TGSPVAGFNA |
| | VLDILKAWDK ATYRQELEMV NLKDKLLKQR EHIYRNFAAG LIKKYAHIVV EDMGLAELAK |
| | TEKSETETND MPNAVKANRQ RASLYSLVEA IRLSAAKVGS YFEKSKAAYS SMTCNVCGHL |
| | NPKTQNIHQS CESCNTMYDV DENAARNFLK GEYINEKVLK QG |

In some embodiments, a nuclease of the present invention is a nuclease having a specified degree of amino acid sequence identity to one or more reference polypeptides, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% sequence identity to the amino acid sequence of any one of SEQ ID NOs: 1-37 and SEQ ID NOs: 221-224. Homology or identity can be determined by amino acid sequence alignment, e.g., using a program such as BLAST, ALIGN, or CLUSTAL, as described herein. In some embodiments, a nuclease having a specified degree of amino acid sequence identity to one or more reference polypeptides retains one or more characteristics, e.g., nuclease activity, as the one or more reference polypeptides.

In some embodiments, a nuclease of the present invention comprises a protein with an amino acid sequence with at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference amino acid sequence. In some embodiments, a nuclease having a specified degree of amino acid sequence identity to one or more reference polypeptides retains one or more characteristics, e.g., nuclease activity, as the reference amino acid sequence.

Also provided is a nuclease of the present invention having enzymatic activity, e.g., nuclease activity, and comprising an amino acid sequence which differs from the amino acid sequences of any one of any one of SEQ ID NOs: 1-37 and SEQ ID NOs: 221-224 by no more than 50, no more than 40, no more than 35, no more than 30, no more than 25, no more than 20, no more than 19, no more than 18, no more than 17, no more than 16, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 amino acid residue(s), when aligned using any of the previously described alignment methods.

In some embodiments, a nuclease of the present invention comprises a RuvC domain In some embodiments, a nuclease of the present invention comprises a split RuvC domain or two or more partial RuvC domains. For example, a nuclease comprises RuvC motifs that are not contiguous with respect to the primary amino acid sequence of the nuclease but form a RuvC domain once the protein folds. In some embodiments, the catalytic residue of a RuvC motif is a glutamic acid residue and/or an aspartic acid residue. For example, the nuclease of SEQ ID NO: 2 comprises the following catalytic residues: D280, E439, D560. See, e.g., Example 1.

In some embodiments, the invention includes an isolated, recombinant, substantially pure, or non-naturally occurring nuclease comprising a RuvC domain, wherein the nuclease has enzymatic activity, e.g., nuclease activity, wherein the nuclease comprises an amino acid sequence having at least about 60%, 65%, 70%, 75%, 80%, 85%, 86%, 87%, 88%, 89%, 90%, 91%, 92%, 93%, 94%, 95%, 96%, 97%, 98%, 99% or 100% sequence identity to any one of SEQ ID NOs: 1-37 and SEQ ID NOs: 221-224.

In some embodiments, a nuclease described herein comprise the consensus sequence shown in FIGS. 1A-1L. In some embodiments, a nuclease described herein comprises a portion of the consensus sequence shown in FIGS. 1A-1L, e.g. a conserved sequence of any one of FIGS. 1A-1L. For example, in some embodiments, a nuclease comprises a sequence set forth as X₁X₂X₃X₄GX₅X₆ (SEQ ID NO: 233), wherein X₁ is V or A or C, X₂ is Y or F, X₃ is K or Q, X₄ is Y or F, X₅ is L or A or M or C or T, and X₆ is I or V or L. In some embodiments, the sequence set forth in SEQ ID NO: 233 is an N-terminal sequence. In some embodiments, a nuclease comprises a sequence set forth as LX₁NX₂LV (SEQ ID NO: 234), wherein X₁ is W or K or R and X₂ is N or T or K or S or D or Q. In some embodiments, the sequence set forth as SEQ ID NO: 234 is an N-terminal sequence. In some embodiments, a nuclease comprises a sequence set forth as FDX₁X₂G (SEQ ID NO: 235), wherein X₁ is G or Y and X₂ is T or S or M. In some embodiments, the sequence set forth as SEQ ID NO: 235 is an N-terminal sequence. In some embodiments, a nuclease comprises a sequence set forth as X₁X₂HR X₃X₄P (SEQ ID NO: 236), wherein X₁ is I or L or V, X₂ is Y or L or M or F, X₃ is P or H or D or E, and X₄ is L or I or V or M. In some embodiments, a nuclease comprises a sequence set forth as GX₁DX₂GX₃R (SEQ ID NO: 237), wherein X₁ is I or L or V, X₂ is I or V or L, and X₃ is F or Y. In some embodiments, a nuclease comprises a sequence set forth as RX₁X₂X₃YR (SEQ ID NO: 238), wherein X₁ is K or Q or E, X₂ is H or D or E, and X₃ is F or V or L or I. In some embodiments, the sequence set forth as SEQ ID NO: 238 is a C-terminal sequence. In some embodiments, a nuclease comprises a sequence set forth as X₁DX₂DX₃NAAX₄N (SEQ ID NO: 239), wherein X₁ is H or Y, X₂ is R or Q or V, X₃ is E or T or I or H or K or Q or D, and X₄ is N or R or I or V or K. In some embodiments, the sequence set forth as SEQ ID NO: 239 is a C-terminal sequence.

In some embodiments, a nuclease described herein comprises an insertion relative to the sequence of any one of SEQ ID NOs: 1-37 and SEQ ID NOs: 221-224. In some embodiments, the insertion comprises one residue to about 10 residues in length (e.g., 1, 2, 3, 4, 5, 6, 7, 8, 9, or 10 residues). In some embodiments, the insertion comprises one or more of a glycine, serine, aspartate, or asparagine residue. In some embodiments, the insertion comprises a one-residue insertion (e.g., one glycine, one serine, one aspartate, or one asparagine). In some embodiments, the insertion comprises a two-residue insertion (e.g., two glycines, two serines, two aspartates, or two asparagines). In some embodiments, the insertion comprises a two-residue insertion comprising at least one glycine. In some embodiments, the insertion comprises a three-residue insertion (e.g., three glycines, three serines, three aspartates, or three asparagines). In some embodiments, the insertion comprises a three-residue insertion comprising at least one glycine. In some embodiments, the insertion comprises a four-residue insertion (e.g., four glycines, four serines, four aspartates, or four asparagines). In some embodiments, the insertion comprises a four-residue insertion comprising at least one glycine. In some embodiments, the insertion comprises a five-residue insertion (e.g., five glycines, five serines, five aspartates, or five asparagines). In some embodiments, the insertion comprises a five-residue insertion comprising at least one glycine. In some embodiments, a nuclease described herein comprises a glycine-glycine, serine-serine, aspartate-aspartate, asparagine-asparagine, glycine-serine, glycine-aspartate, glycine-asparagine, serine-glycine, aspartate-glycine, or asparagine-glycine insertion relative to the sequence of any one of SEQ ID NOs: 1-37 and SEQ ID NOs: 221-224.

### Biochemical Characteristics

In some embodiments, the biochemistry of a nuclease described herein is analyzed using one or more assays. In some embodiments, the biochemical characteristics of a nuclease of the present invention are analyzed in vitro using a purified nuclease incubated with an RNA guide (e.g., a mature crRNA) and a target DNA molecule, as described in Examples 4 and 5. In some embodiments, the biochemical characteristics of a nuclease of the present invention are analyzed in vitro using a fluorescence depletion assay, as described in Example 6. In some embodiments, the biochemical characteristics of a nuclease of the present invention are analyzed in mammalian cells, as described in Example 7.

Described herein are compositions and methods relating to a nuclease of the present invention. The compositions and methods are based, in part, on the observation that cloned and expressed effectors of the present invention have nuclease activity.

In some embodiments, a nuclease and an RNA guide as described herein form a complex (e.g., an RNP). In some embodiments, the complex includes other components. In some embodiments, the complex is activated upon binding to a nucleic acid substrate that has complementarity to a spacer sequence in the RNA guide (e.g., a target nucleic acid). In some embodiments, the target nucleic acid is a double-stranded DNA (dsDNA). In some embodiments, the target nucleic acid is a single-stranded DNA (ssDNA). In some embodiments, the target nucleic acid is a single-stranded RNA (ssRNA). In some embodiments, the target nucleic acid is a double-stranded RNA (dsRNA). In some embodiments, the sequence-specificity requires a complete match of the spacer sequence in the RNA guide to the target substrate. In other embodiments, the sequence specificity requires a partial (contiguous or non-contiguous) match of the spacer sequence in the RNA guide to the target substrate.

In some embodiments, the complex becomes activated upon binding to the target substrate. In some embodiments, the activated complex exhibits “multiple turnover” activity, whereby upon acting on (e.g., cleaving) the target nucleic acid, the activated complex remains in an activated state. In some embodiments, the activated complex exhibits “single turnover” activity, whereby upon acting on the target nucleic acid, the complex reverts to an inactive state.

In some embodiments, a nuclease described herein binds to a target nucleic acid at a sequence defined by the region of complementarity between the RNA guide and the target nucleic acid. In some embodiments, the PAM sequence of a nuclease described herein is located directly upstream of the target sequence of the target nucleic acid (e.g., directly 5′ of the target sequence). In some embodiments, the PAM sequence of a nuclease described herein is located directly 5′ of the non-complementary strand (e.g., non-target strand) of the target nucleic acid. As used herein, the “complementary strand” hybridizes to the RNA guide. As used herein, the “non-complementary strand” does not directly hybridize to the RNA.

In some embodiments, a nuclease of the present invention targets a sequence adjacent to a PAM, wherein the PAM comprises a nucleotide sequence set forth as 5′-CN-3′, 5′-CCN-3′, 5′-NCN-3′, 5′-NCCN-3′, or 5′-NNCN-3′, wherein “N” is any nucleobase. For example, in some embodiments, the nuclease of SEQ ID NO: 2 recognizes a PAM sequence of 5′-CCN-3′ (e.g., 5′-CCA-3′) or 5′-NCCN-3′. In some embodiments, a nuclease of the present invention targets a sequence adjacent to a PAM, wherein the PAM comprises a nucleotide sequence set forth in Table 2, wherein “D” is A or G or T, “H” A or C or T, “N” is any nucleobase, “R” is A or G, “V” is A or C or G, and “Y” is C or T. In some embodiments, a nuclease of the present invention (e.g., the nuclease of SEQ ID NO: 2) targets a sequence adjacent to a PAM sequence, wherein the PAM comprises a nucleotide sequence set forth as 5′-CCA-3′, 5′-CCC-3′, 5′-CCT-3′, 5′-CCG-3′, 5′-ACCG-3′, 5′-CCCA-3′, 5′-CCCG-3′, 5′-TCCA-3′, or 5′-TCCT-3′.

TABLE 2
PAM sequences of the present invention.

| Effector SEQ ID NO | PAM Sequence |
|---|---|
| 2 | 5′-CCN-3′ |
| | 5-NCCN-3′ |
| 5 | 5′-CCN-3′ |
| 6 | 5′-RYCN-3′ |
| 7 | 5′-NCN-3′ |
| | 5′-CCN-3′ |
| | 5′-HCN-3′ |
| 10 | 5′-CCN-3′ |
| | 5′-RCCN-3′ |
| 11 | 5′-NCCN-3′ |
| | 5′-ACCN-3′ |
| 13 | 5′-NNCN-3′ |
| | 5′-TNCN-3′ |
| | 5′-HNCN-3′ |
| | 5′-HNCV-3′ |
| | 5′-HNCR-3′ |
| 16 | 5′-NCN-3′ |
| 18 | 5′-GCCN-3′ |
| | 5′-GTTN-3′ |
| | 5′-GYYN-3′ |
| | 5′-DCCN-3′ |
| | 5′-DTTN-3′ |
| | 5′-DYYN-3′ |
| 20 | 5′-NCCN-3′ |
| 27 | 5′-NCCN-3′ |
| | 5′-RCCR-3′ |
| 31 | 5′-GCCN-3′ |
| | 5′-GTTN-3′ |
| | 5′-GYYN-3′ |
| | 5′-DCCN-3′ |
| | 5′-DTTN-3′ |
| | 5′-DYYN-3′ |
| 35 | 5′-CCN-3′ |
| 37 | 5′-CN-3′ |

In some embodiments, a nuclease described herein cleaves ssDNA. In some embodiments, a nuclease described herein cleaves dsDNA. In some embodiments, a nuclease described herein is a nickase (e.g., the nuclease cleaves one strand of a double-stranded target nucleic acid).

In some embodiments, a nuclease of the present invention has enzymatic activity, e.g., nuclease activity, over a broad range of pH conditions. In some embodiments, the nuclease has enzymatic activity, e.g., nuclease activity, at a pH of from about 3.0 to about 12.0. In some embodiments, the nuclease has enzymatic activity at a pH of from about 4.0 to about 10.5. In some embodiments, the nuclease has enzymatic activity at a pH of from about 5.5 to about 8.5. In some embodiments, the nuclease has enzymatic activity at a pH of from about 6.0 to about 8.0. In some embodiments, the nuclease has enzymatic activity at a pH of about 7.0.

In some embodiments, a nuclease of the present invention has enzymatic activity, e.g., nuclease activity, at a temperature range of from about 10° C. to about 100° C. In some embodiments, a nuclease of the present invention has enzymatic activity at a temperature range from about 20° C. to about 90° C. In some embodiments, a nuclease of the present invention has enzymatic activity at a temperature of about C to about 25° C. or at a temperature of about 37° C.

In some embodiments wherein a nuclease of the present invention induces double-stranded breaks or single-stranded breaks in a target nucleic acid, (e.g. genomic DNA), the double-stranded break can stimulate cellular endogenous DNA-repair pathways, including Homology Directed Recombination (HDR), Non-Homologous End Joining (NHEJ), or Alternative Non-Homologues End-Joining (A-NHEJ). NHEJ can repair cleaved target nucleic acid without the need for a homologous template. This can result in deletion or insertion of one or more nucleotides at the target locus. HDR can occur with a homologous template, such as the donor DNA. The homologous template can comprise sequences that are homologous to sequences flanking the target nucleic acid cleavage site. In some cases, HDR can insert an exogenous polynucleotide sequence into the cleave target locus. The modifications of the target DNA due to NHEJ and/or HDR can lead to, for example, mutations, deletions, alterations, integrations, gene correction, gene replacement, gene tagging, transgene knock-in, gene disruption, and/or gene knock-outs.

In some embodiments, binding of a nuclease/RNA guide complex to a target locus in a cell recruits one or more endogenous cellular molecules or pathways other than DNA repair pathways to modify the target nucleic acid. In some embodiments, binding of a nuclease/RNA guide complex blocks access of one or more endogenous cellular molecules or pathways to the target nucleic acid, thereby modifying the target nucleic acid. For example, binding of a nuclease/RNA guide complex may block endogenous transcription or translation machinery to decrease the expression of the target nucleic acid.

### Variants

In some embodiments, the present invention includes variants of a nuclease described herein. In some embodiments, a nuclease described herein can be mutated at one or more amino acid residues to modify one or more functional activities. For example, in some embodiments, a nuclease of the present invention is mutated at one or more amino acid residues to modify its nuclease activity (e.g., cleavage activity). For example, in some embodiments, a nuclease may comprise one or more mutations that increase the ability of the nuclease to cleave a target nucleic acid. In some embodiments, a nuclease is mutated at one or more amino acid residues to modify its ability to functionally associate with an RNA guide. In some embodiments, a nuclease is mutated at one or more amino acid residues to modify its ability to functionally associate with a target nucleic acid.

In some embodiments, a variant nuclease has a conservative or non-conservative amino acid substitution, deletion or addition. In some embodiments, the variant nuclease has a silent substitution, deletion or addition, or a conservative substitution, none of which alter the polypeptide activity of the present invention. Typical examples of the conservative substitution include substitution whereby one amino acid is exchanged for another, such as exchange among aliphatic amino acids Ala, Val, Leu and Ile, exchange between hydroxyl residues Ser and Thr, exchange between acidic residues Asp and Glu, substitution between amide residues Asn and Gln, exchange between basic residues Lys and Arg, and substitution between aromatic residues Phe and Tyr. In some embodiments, one or more residues of a nuclease disclosed herein are mutated to an Arg residue. In some embodiments, one or more residues of a nuclease disclosed herein are mutated to a Gly residue.

A variety of methods are known in the art that are suitable for generating modified polynucleotides that encode variant nucleases of the invention, including, but not limited to, for example, site-saturation mutagenesis, scanning mutagenesis, insertional mutagenesis, deletion mutagenesis, random mutagenesis, site-directed mutagenesis, and directed-evolution, as well as various other recombinatorial approaches. Methods for making modified polynucleotides and proteins (e.g., nucleases) include DNA shuffling methodologies, methods based on non-homologous recombination of genes, such as ITCHY (See, Ostermeier et al., 7:2139-44 [1999]), SCRACHY (See, Lutz et al. 98:11248-53 [2001]), SHIPREC (See, Sieber et al., 19:456-60 [2001]), and NRR (See, Bittker et al., 20:1024-9 [2001]; Bittker et al., 101:7011-6 [2004]), and methods that rely on the use of oligonucleotides to insert random and targeted mutations, deletions and/or insertions (See, Ness et al., 20:1251-5 [2002]; Coco et al., 20:1246-50 [2002]; Zha et al., 4:34-9 [2003]; Glaser et al., 149:3903-13 [1992]).

In some embodiments, a nuclease of the present invention comprises an alteration at one or more (e.g., several) amino acids in the nuclease, wherein at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 162, 164, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, or more.

As used herein, a “biologically active portion” is a portion that maintains the function (e.g. completely, partially, minimally) of a nuclease (e.g., a “minimal” or “core” domain). In some embodiments, a nuclease fusion protein is useful in the methods described herein. Accordingly, in some embodiments, a nucleic acid encoding the fusion nuclease is described herein. In some embodiments, all or a portion of one or more components of the nuclease fusion protein are encoded in a single nucleic acid sequence.

Although the changes described herein may be one or more amino acid changes, changes to a nuclease may also be of a substantive nature, such as fusion of polypeptides as amino- and/or carboxyl-terminal extensions. For example, nuclease may contain additional peptides, e.g., one or more peptides. Examples of additional peptides may include epitope peptides for labelling, such as a polyhistidine tag (His-tag), Myc, and FLAG. In some embodiments, a nuclease described herein can be fused to a detectable moiety such as a fluorescent protein (e.g., green fluorescent protein (GFP) or yellow fluorescent protein (YFP)).

A nuclease described herein can be modified to have diminished nuclease activity, e.g., nuclease inactivation of at least 50%, at least 60%, at least 70%, at least 80%, at least 90%, at least 95%, at least 97%, or 100%, as compared to a reference nuclease. Nuclease activity can be diminished by several methods known in the art, e.g., introducing mutations into the RuvC domain (e.g, one or more catalytic residues of the RuvC domain). In a non-limiting example, a variant of SEQ ID NO: 2 comprising a mutation in residue D280, residue E439, and/or residue D560 demonstrates diminished or no nuclease activity.

In some embodiments, the nuclease described herein can be self-inactivating. See, Epstein et al., “Engineering a Self-Inactivating CRISPR System for AAV Vectors,” Mol. Ther., 24 (2016): S50, which is incorporated by reference in its entirety.

Nucleic acid molecules encoding the nucleases described herein can further be codon-optimized. The nucleic acid can be codon-optimized for use in a particular host cell, such as a bacterial cell or a mammalian cell.

### Targeting Moiety

In some embodiments, the composition described herein comprises a targeting moiety.

The targeting moiety may be substantially identical to a reference nucleic acid sequence if the targeting moiety comprises a sequence having least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence. The percent identity between two such nucleic acids can be determined manually by inspection of the two optimally aligned nucleic acid sequences or by using software programs or algorithms (e.g., BLAST, ALIGN, CLUSTAL) using standard parameters. One indication that two nucleic acid sequences are substantially identical is that the two nucleic acid molecules hybridize to each other under stringent conditions (e.g., within a range of medium to high stringency).

In some embodiments, the targeting moiety has at least about 60%, least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to the reference nucleic acid sequence.

### RNA Guide Sequence

In some embodiments, the targeting moiety comprises, or is, an RNA guide sequence. In some embodiments, the RNA guide sequence directs a nuclease described herein to a particular nucleic acid sequence. Those skilled in the art reading the below examples of particular kinds of RNA guide sequences will understand that, in some embodiments, an RNA guide sequence is site-specific. That is, in some embodiments, an RNA guide sequence associates specifically with one or more target nucleic acid sequences (e.g., specific DNA or genomic DNA sequences) and not to non-targeted nucleic acid sequences (e.g., non-specific DNA or random sequences).

In some embodiments, the composition as described herein comprises an RNA guide sequence that associates with a nuclease described herein and directs a nuclease to a target nucleic acid sequence (e.g., DNA). The RNA guide sequence may associate with a nucleic acid sequence and alter functionality of a nuclease (e.g., alters affinity of the nuclease to a molecule, e.g., at least 10%, 15%, 20%, 25%, 30%, 35%, 40%, 45%, 50%, 55%, 60%, 65%, 70%, 75%, 80%, 85%, 90%, 95%, or more).

The RNA guide sequence may target (e.g., associate with, be directed to, contact, or bind) one or more nucleotides of a sequence, e.g., a site-specific sequence or a site-specific target. In some embodiments, a nuclease (e.g., a nuclease plus an RNA guide) is activated upon binding to a nucleic acid substrate that is complementary to a spacer sequence in the RNA guide (e.g., a sequence-specific substrate or target nucleic acid).

In some embodiments, an RNA guide sequence comprises a spacer sequence. In some embodiments, the spacer sequence of the RNA guide sequence may be generally designed to have a length of between 16-24 nucleotides (e.g., 18, 19, 20, or 21 nucleotides) and be complementary to a specific nucleic acid sequence. In some embodiments, the length of the spacer is a length shown in Table 3. In some particular embodiments, the RNA guide sequence may be designed to be complementary to a specific DNA strand, e.g., of a genomic locus. In some embodiments, the spacer sequence is designed to be complementary to a specific DNA strand, e.g., of a genomic locus.

TABLE 3
Exemplary spacer lengths for mature crRNAs.

| Effector SEQ ID NO | Spacer Length |
|---|---|
| 2 | 19 nucleotides |
| 5 | 19 nucleotides |
| 7 | 18 nucleotides |
| 13 | 19 nucleotides |
| 14 | 19 nucleotides |
| 16 | 18 nucleotides |
| 18 | 18 nucleotides |
| 19 | 16 nucleotides |
| 21 | 19 nucleotides |
| 27 | 19-20 nucleotides |
| 28 | 21-22 nucleotides |
| 31 | 18 nucleotides |
| 35 | 19 nucleotides |
| 37 | 18 nucleotides |

In certain embodiments, the RNA guide sequence includes, consists essentially of, or comprises a direct repeat sequence linked to a sequence or spacer sequence. In some embodiments, the RNA guide sequence includes a direct repeat sequence and a spacer sequence or a direct repeat-spacer-direct repeat sequence. In some embodiments, the RNA guide sequence includes a truncated direct repeat sequence and a spacer sequence, which is typical of processed or mature crRNA. In some embodiments, a nuclease forms a complex with the RNA guide sequence, and the RNA guide sequence directs the complex to associate with site-specific target nucleic acid that is complementary to at least a portion of the RNA guide sequence.

In some embodiments, the RNA guide sequence comprises a sequence, e.g., RNA sequence, at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a DNA sequence. In some embodiments, the RNA guide sequence comprises a sequence at least 80%, at least 90%, at least 95%, at least 96%, at least 97%, at least 98%, at least 99% complementary to a target nucleic acid sequence. In some embodiments, the RNA guide sequence comprises a sequence at least 80%, at least 90%, at least 95%, at least 96%, at least 97%, at least 98%, at least 99% complementary to a genomic sequence. In some embodiments, the RNA guide sequence comprises a sequence complementary to or a sequence comprising at least 80%, at least 90%, at least 95%, at least 96%, at least 97%, at least 98%, at least 99% complementarity to a genomic sequence.

In some embodiments, a nuclease described herein includes one or more (e.g., two, three, four, five, six, seven, eight, or more) RNA guide sequences, e.g., RNA guides.

In some embodiments, the RNA guide has an architecture similar to, for example International Publication Nos. WO 2014/093622 and WO 2015/070083, the entire contents of each of which are incorporated herein by reference.

In some embodiments, an RNA guide sequence of the present invention comprises a direct repeat sequence having 80, 85, 90, 91, 92, 93, 94, 95, 96, 97, 98, 99 or 100% identity the direct repeat sequences of Table 4. In some embodiments, an RNA guide of the present invention comprises a direct repeat sequence having greater than 80, 85, 90, 91, 92, 93, 94, 95, 96, 97, 98, 99 or 100% identity to the direct repeat sequences of Table 4.

TABLE 4
Pre-crRNA direct repeat sequences.

| Effector | |
|---|---|
| SEQ ID NO | Direct Repeat Sequence |
| 1 | GUUUUAGAACCCUGUUGAGUGGGCAUAAACUCAAACU (SEQ ID NO: 38) |
| | AGUUUGAGUUUAUGCCCACUCAACAGGGUUCUAAAAC (SEQ ID NO: 39) |
| 2 | GUAUAACGACCCUGCGAAGUGGGGUGUAACUUCGAC (SEQ ID NO: 40) |
| | GUCGAAGUUACACCCCACUUCGCAGGGUCGUUAUAC (SEQ ID NO: 41) |
| 3 | GAUUAAGGCCCCUGUGUAGUGGGGUG (SEQ ID NO: 42) |
| | CACCCCACUACACAGGGGCCUUAAUC (SEQ ID NO: 43) |
| 4 | CCUAUAGAACCCUGCCAAGUGGGUAGUGACUUGGAC (SEQ ID NO: 44) |
| | GUCCAAGUCACUACCCACUUGGCAGGGUUCUAUAGG (SEQ ID NO: 45) |
| 5 | GUAUAACGACCCUGCGAAGUGGGGUGUAACUUCGAC (SEQ ID NO: 46) |
| | GUCGAAGUUACACCCCACUUCGCAGGGUCGUUAUAC (SEQ ID NO: 47) |
| 6 | GUGUAAGGCCCCUGUGCAUUGGGGUGUAAAUGCAAC (SEQ ID NO: 48) |
| | GUUGCAUUUACACCCCAAUGCACAGGGGCCUUACAC (SEQ ID NO: 49) |
| 7 | CCUUAAGGCCCCUGUCUAGUGGGGUGUAACUAGAAC (SEQ ID NO: 50) |
| | GUUCUAGUUACACCCCACUAGACAGGGGCCUUAAGG (SEQ ID NO: 51) |
| 8 | AGUAGAACCCUGUCGCUUGGGUGGUAAAGCGAAC (SEQ ID NO: 52) |
| | GUUCGCUUUACCACCCAAGCGACAGGGUUCUACU (SEQ ID NO: 53) |
| 9 | AUUGUAAGACCCUGUUGGAUGGGGUGUGA (SEQ ID NO: 54) |
| | UCACACCCCAUCCAACAGGGUCUUACAAU (SEQ ID NO: 55) |
| 10 | CUUUAAGGACCCUGUACGUUGGGGUGUAAACGUAAC (SEQ ID NO: 56) |
| | GUUACGUUUACACCCCAACGUACAGGGUCCUUAAAG (SEQ ID NO: 57) |
| 11 | UUUAAGGACCCUGUACGUUGGGGUGUAAACGUAAC (SEQ ID NO: 58) |
| | GUUACGUUUACACCCCAACGUACAGGGUCCUUAAA (SEQ ID NO: 59) |
| 12 | GUGAUAGUGCCUGCUCAGUGGCUUAGU (SEQ ID NO: 60) |
| | ACUAAGCCACUGAGCAGGCACUAUCAC (SEQ ID NO: 61) |
| 13 | GCUUGAGAACCCUGUCGAUUGGGUGGUGAAUCGAAC (SEQ ID NO: 62) |
| | GUUCGAUUCACCACCCAAUCGACAGGGUUCUCAAGC (SEQ ID NO: 63) |
| 14 | GUAUAACGACCCUGCGAAGUGGGGUGUAACUUCGAC (SEQ ID NO: 64) |
| | GUCGAAGUUACACCCCACUUCGCAGGGUCGUUAUAC (SEQ ID NO: 65) |
| 15 | GUUGCAUUUACACCCCAAUGCACAGGGGCCUUAGAC (SEQ ID NO: 66) |
| | GUCUAAGGCCCCUGUGCAUUGGGGUGUAAAUGCAAC (SEQ ID NO: 220) |
| 16 | GUUAUAGGACCCUGCCGGGUGGGGUGUGACCCGGAC (SEQ ID NO: 67) |
| | GUCCGGGUCACACCCCACCCGGCAGGGUCCUAUAAC (SEQ ID NO: 68) |
| 17 | GUUGCAUUUACACCCCAAUGCACAGGGGCCUUAGAC (SEQ ID NO: 69) |
| | GUCUAAGGCCCCUGUGCAUUGGGGUGUAAAUGCAAC (SEQ ID NO: 70) |
| 18 | AUUUAAUGACCCUGCGUGUUGGGGUGUGAACACGAC (SEQ ID NO: 71) |
| | GUCGUGUUCACACCCCAACACGCAGGGUCAUUAAAU (SEQ ID NO: 72) |
| 19 | UCAGGUAUCUCGACCCUACGGAUGGGGGGAACAUCCGGC (SEQ ID NO: 73) |
| | GCCGGAUGUUCCCCCCAUCCGUAGGGUCGAGAUACCUGA (SEQ ID NO: 74) |
| 20 | CUUUAAGGACCCUGUACGUUGGGGUGUAAACGUAAC (SEQ ID NO: 75) |
| | GUUACGUUUACACCCCAACGUACAGGGUCCUUAAAG (SEQ ID NO: 76) |
| 21 | GUAUAACGACCCUGCGAAGUGGGGUGUAACUUCGAC (SEQ ID NO: 77) |
| | GUCGAAGUUACACCCCACUUCGCAGGGUCGUUAUAC (SEQ ID NO: 78) |
| 22 | CUUUAAGGACCCUGUACGUUGGGGUGUAAA (SEQ ID NO: 79) |
| | UUUACACCCCAACGUACAGGGUCCUUAAAG (SEQ ID NO: 80) |
| 23 | GUUGCAUUUACACCCCAAUGCACAGGGGCCUUAGACA (SEQ ID NO: 81) |
| | UGUCUAAGGCCCCUGUGCAUUGGGGUGUAAAUGCAAC (SEQ ID NO: 82) |
| 24 | CUUUAAGGACCCUGUACGUUGGGGUGUAAACGUAAC (SEQ ID NO: 83) |
| | GUUACGUUUACACCCCAACGUACAGGGUCCUUAAAG (SEQ ID NO: 84) |
| 25 | GGCCCCUGUGCAUUGGGGUGUAAA (SEQ ID NO: 85) |
| | UUUACACCCCAAUGCACAGGGGCC (SEQ ID NO: 86) |
| 26 | CUUUAAGGACCCUGUACGUUGGGGUGUAAA (SEQ ID NO: 87) |
| | UUUACACCCCAACGUACAGGGUCCUUAAAG (SEQ ID NO: 88) |
| 27 | CUUUAAGGACCCUGUACGUUGGGGUGUAAACGUAAC (SEQ ID NO: 89) |
| | GUUACGUUUACACCCCAACGUACAGGGUCCUUAAAG (SEQ ID NO: 90) |
| 28 | CUUUAAGGACCCUGUACGUUGGGGUGUAAACGUAAC (SEQ ID NO: 91) |
| | GUUACGUUUACACCCCAACGUACAGGGUCCUUAAAG (SEQ ID NO: 92) |
| 29 | GAGCGGUUAACAGGGUGUCGAUAUAGAUU (SEQ ID NO: 93) |
| | AAUCUAUAUCGACACCCUGUUAACCGCUC (SEQ ID NO: 94) |
| 30 | GAUUAAGGCCCCUGUGUAGUGGGGUGUAACUACAAC (SEQ ID NO: 95) |
| | GUUGUAGUUACACCCCACUACACAGGGGCCUUAAUC (SEQ ID NO: 96) |
| 31 | AUUUAAUGACCCUGCGUGUUGGGGUGUGAACACGAC (SEQ ID NO: 97) |
| | GUCGUGUUCACACCCCAACACGCAGGGUCAUUAAAU (SEQ ID NO: 98) |
| 32 | UAAGGACCCUGUACGUUGGGGUGUAAACGUAAC (SEQ ID NO: 99) |
| | GUUACGUUUACACCCCAACGUACAGGGUCCUUA (SEQ ID NO: 100) |
| 33 | GGCCCCUGUGCAUUGGGGUGUAAA (SEQ ID NO: 101) |
| | UUUACACCCCAAUGCACAGGGGCC (SEQ ID NO: 102) |
| 34 | GUUGCAUUUACACCCCAAUGCACAGGGGCCUUAGAC (SEQ ID NO: 103) |
| | GUCUAAGGCCCCUGUGCAUUGGGGUGUAAAUGCAAC (SEQ ID NO: 104) |
| 35 | GUAUAACGACCCUGCGAAGUGGGGUGUAACUUCGAC (SEQ ID NO: 105) |
| | GUCGAAGUUACACCCCACUUCGCAGGGUCGUUAUAC (SEQ ID NO: 106) |
| 36 | GUUGCAUUUACACCCCAAUGCACAGGGGCCUUAGAC (SEQ ) NO: 107) |
| | GUCUAAGGCCCCUGUGCAUUGGGGUGUAAAUGCAAC (SEQ ID NO: 108) |
| 37 | CCUUAAGGCCCCUGUCUAGUGGGGUGUAACUAGAAC (SEQ ID NO: 109) |
| | GUUCUAGUUACACCCCACUAGACAGGGGCCUUAAGG (SEQ ID NO: 110) |
| 221 | CUUUAAGGACCCUGUACGUUGGGGUGUAAACGUAAC (SEQ ID NO: 225) |
| | GUUACGUUUACACCCCAACGUACAGGGUCCUUAAAG (SEQ ID NO: 226) |
| 222 | GAUUAAGGCCCCUGUGCAUCGGGGUGUAAAUGCAAC (SEQ ID NO: 227) |
| | GUUGCAUUUACACCCCGAUGCACAGGGGCCUUAAUC (SEQ ID NO: 228) |
| 223 | GGUAUCAUGACCCUACGGAUGGGGGG (SEQ ID NO: 229) |
| | CCCCCCAUCCGUAGGGUCAUGAUACC (SEQ ID NO: 230) |
| 224 | AGUAGAACCCUGUCGCUUGGGCGGUAAAGCGAAC (SEQ ID NO: 231) |
| | GUUCGCUUUACCGCCCAAGCGACAGGGUUCUACU (SEQ ID NO: 232) |

In some embodiments, a CRISPR-associated protein and an RNA guide (e.g., an RNA guide comprising a direct repeat and a spacer) form a complex. In some embodiments, a CRISPR-associated protein and an RNA guide (e.g., an RNA guide comprising direct repeat-spacer-direct repeat sequence or pre-crRNA) form a complex. In some embodiments, the complex binds a target nucleic acid. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 1, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 38 or SEQ ID NO: 39. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 40 or SEQ ID NO: 41. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 3, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 42 or SEQ ID NO: 43. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 44 or SEQ ID NO: 45. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 4, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 44 or SEQ ID NO: 45. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 5, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 46 or SEQ ID NO: 47. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 6, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 48 or SEQ ID NO: 49. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 7, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 50 or SEQ ID NO: 51. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 8, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 52 or SEQ ID NO: 53. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 9, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 54 or SEQ ID NO: 55. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 10, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 56 or SEQ ID NO: 57. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 11, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 58 or SEQ ID NO: 59. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 12, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 60 or SEQ ID NO: 61. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 13, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 62 or SEQ ID NO: 63. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 14, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 64 or SEQ ID NO: 65. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 15, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 66 or SEQ ID NO: 220. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 16, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 67 or SEQ ID NO: 68. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 17, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 69 or SEQ ID NO: 70. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 71 or SEQ ID NO: 72. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 19, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 73 or SEQ ID NO: 74. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 20, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 75 or SEQ ID NO: 76. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 22, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 79 or SEQ ID NO: 80. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 23, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 81 or SEQ ID NO: 82. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 24, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 83 or SEQ ID NO: 84. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 25, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 85 or SEQ ID NO: 86. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 26, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 87 or SEQ ID NO: 88. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 89 or SEQ ID NO: 90. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 28, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 91 or SEQ ID NO: 92. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 29, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 93 or SEQ ID NO: 94. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 30, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 95 or SEQ ID NO: 96. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 97 or SEQ ID NO: 98. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 32, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 99 or SEQ ID NO: 100. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 33, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 101 or SEQ ID NO: 102. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 34, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 103 or SEQ ID NO: 104. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 35, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 105 or SEQ ID NO: 106. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 36, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 107 or SEQ ID NO: 108. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 37, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 109 or SEQ ID NO: 110.

In some embodiments, a CRISPR-associated protein and an RNA guide (e.g., an RNA guide comprising a direct repeat and a spacer) form a complex. In some embodiments, a CRISPR-associated protein and an RNA guide (e.g., an RNA guide comprising direct repeat-spacer-direct repeat sequence or a pre-crRNA) form a complex. In some embodiments, the complex binds a target nucleic acid. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 40. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 5, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 46. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 6, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 48. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 7, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 50. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 10, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 56. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 11, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 58. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 13, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 62. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 15, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 66. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 16, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 67. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 17, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 69. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 71. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 19, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 73. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 20, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 75. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 23, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 81. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 89. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 97. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 34, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 103. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 35, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 105. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 36, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 107. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 37, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 109. In some embodiments, the complex binds a target nucleic acid. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 221, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 225 or SEQ ID NO: 226. In some embodiments, the complex binds a target nucleic acid. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 222, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 227 or SEQ ID NO: 228. In some embodiments, the complex binds a target nucleic acid. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 223, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 229 or SEQ ID NO: 230. In some embodiments, the complex binds a target nucleic acid. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 224, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 231 or SEQ ID NO: 232.

In some embodiments, an RNA guide sequence of the present invention comprises a direct repeat sequence having 80, 85, 90, 91, 92, 93, 94, 95, 96, 97, 98, 99 or 100% identity the direct repeat sequences of Table 5. In some embodiments, an RNA guide of the present invention comprises a direct repeat sequence having greater than 80, 85, 90, 91, 92, 93, 94, 95, 96, 97, 98, 99 or 100% identity to the direct repeat sequences of Table 5.

TABLE 5
Mature crRNA direct repeat sequences.

| Effector | |
|---|---|
| SEQ ID NO | Direct repeat sequence |
| 2 | AUAACGACCCUGCGAAGUGGGGUGUAACUUCGAC (SEQ ID NO: 111) |
| 5 | AUAACGACCCUGCGAAGUGGGGUGUAACUUCGAC (SEQ ID NO: 112) |
| 7 | UUAAGGCCCCUGUCUAGUGGGGUGUAACUAGAAC (SEQ ID NO: 113) |
| 13 | CUUGAGAACCCUGUCGAUUGGGUGGUGAAUCGAAC (SEQ ID NO: 114) |
| 14 | AUAACGACCCUGCGAAGUGGGGUGUAACUUCGAC (SEQ ID NO: 115) |
| 16 | AUAGGACCCUGCCGGGUGGGGUGUGACCCGGAC (SEQ ID NO: 116) |
| 18 | UUUAAUGACCCUGCGUGUUGGGGUGUGAACACGAC (SEQ ID NO: 117) |
| 19 | UAUCUCGACCCUACGGAUGGGGGGAACAUCCGGC (SEQ ID NO: 118) |
| 21 | AUAACGACCCUGCGAAGUGGGGUGUAACUUCGAC (SEQ ID NO: 119) |
| 27 | UUUAAGGACCCUGUACGUUGGGGUGUAAACGUAAC (SEQ ID NO: 120) |
| | UUUAAGGACCCUGUACGUUGGGGUGUAAACGUAAC (SEQ ID NO: 121) |
| 28 | UUUAAGGACCCUGUACGUUGGGGUGUAAACGUAAC (SEQ ID NO: 122) |
| 31 | UUUAAUGACCCUGCGUGUUGGGGUGUGAACACGAC (SEQ ID NO: 123) |
| 35 | AUAACGACCCUGCGAAGUGGGGUGUAACUUCGAC (SEQ ID NO: 124) |
| 37 | UUAAGGCCCCUGUCUAGUGGGGUGUAACUAGAAC (SEQ ID NO: 125) |
| | UAAGGCCCCUGUCUAGUGGGGUGUAACUAGAAC (SEQ ID NO: 126) |

In some embodiments, a CRISPR-associated protein and an RNA guide (e.g., an RNA guide comprising a direct repeat and a spacer) form a complex. In some embodiments, a CRISPR-associated protein and an RNA guide (e.g., an RNA guide comprising direct repeat-spacer or a mature crRNA) form a complex. In some embodiments, the complex binds a target nucleic acid. In some embodiments, the 20 CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 2, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 111. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 5, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 112. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 7, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 113. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 13, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 114. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 14, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 115. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 16, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 116. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 18, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 117. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 19, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 118. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 21, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 119. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 27, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 120 or SEQ ID NO: 121. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 28, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 122. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 31, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 123. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 35, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 124. In some embodiments, the CRISPR-associated protein comprises an amino acid sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the amino acid sequence of SEQ ID NO: 37, and the direct repeat sequence comprises a nucleotide sequence that is at least 80% (e.g., 81%, 82%, 83%, 84%, 85%, 86%, 87%, 88%, 89%, 90%, 91%, 92%, 93%, 94%, 95%, 96%, 97%, 98%, 99% or 100%) identical to the nucleotide sequence of SEQ ID NO: 125 or SEQ ID NO: 126.

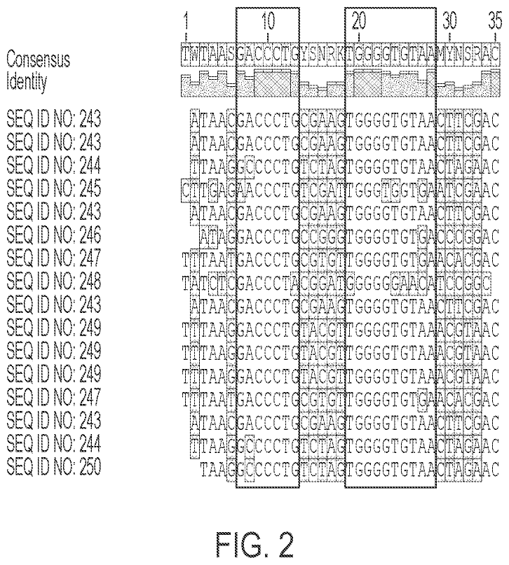

In some embodiments, an RNA guide comprises a direct repeat sequence set forth in FIG. 2. For example, in some embodiments, the RNA guide comprises a direct repeat of the consensus sequence shown in FIG. 2 or a portion of the consensus sequence shown in FIG. 2. For example, in some embodiments, an RNA guide comprises a direct repeat having a sequence set forth as X₁X₂CCCTX₃, wherein X₁ is G or A, X₂ is A or C, and X₃ is G or A. In some embodiments, an RNA guide comprises a direct repeat having a sequence set forth as X₁GGGX₂X₃X₄X₅X₆A, wherein X₁ is T or G, X₂ is T or G, X₃ is T or G, X₄ is A or G, X₅ is T or A, and X₆ is A or G or C (SEQ ID NO: 242).

In some embodiments, an RNA guide described herein comprises a uracil (U). In some embodiments, an RNA guide described herein comprises a thymine (T). In some embodiments, a direct repeat sequence of an RNA guide described herein comprises a uracil (U). In some embodiments, a direct repeat sequence of an RNA guide described herein comprises a thymine (T). In some embodiments, a direct repeat sequence according to Table 4 or Table 5 comprises a sequence comprising a uracil, in one or more places indicated as thymine in the corresponding sequences in Table 4 or Table 5.

In some embodiments, an RNA guide of the present invention optionally comprises a tracrRNA sequence having 80, 85, 90, 91, 92, 93, 94, 95, 96, 97, 98, 99 or 100% identity a tracrRNA sequence of Table 6. In some embodiments, the targeting moiety of the present invention comprises a direct repeat sequence having greater than 80, 85, 90, 91, 92, 93, 94, 95, 96, 97, 98, 99 or 100% identity to a tracrRNA sequence of Table 6. In some embodiments, an RNA guide of the present invention does not comprise a tracrRNA sequence, e.g., a tracrRNA sequence of Table 6. In some embodiments, a nuclease of the present invention, e.g., a nuclease of any one of SEQ ID NOs: 1-37 or SEQ ID NOs: 221-224, does not require a tracrRNA sequence, e.g., a tracrRNA sequence of Table 6, to have activity (e.g., nuclease activity).

TABLE 6
Optional TracrRNA sequences.

| Effector | |
|---|---|
| SEQ ID | |
| NO | TracrRNA sequences |
| 1 | UUUGGAAAGCGUACUGGCUGAAUUUAUAAGCCAGAACGAAAUUCAGUGCGAUGCACAG |
| | GCGAUUAAAUCAUUUGUAGUUGGAUUA (SEQ ID NO: 127) |
| | AUUCAGUGCGAUGCACAGGCGAUUAAAUCAUUUGUAGUUGGAUUA (SEQ ID NO: |
| | 128) |
| 2 | AUUUCCCGUAUCAGGAAAGGUCGUUCAGUUCUCACGUUUGCACUGGAAC |
| | (SEQ ID NO: 129) |
| 3 | ACUAUUAUAAAAUUUUGAAGUCAUUUAAAAAUGAGAACCAGAGUUUACAAAUACGGUC |
| | UUGUUCCUAUCGGCUAUCCACCACAGGAAGCUGUAGAUGAACUU (SEQ ID NO: |
| | 130) |
| | AUUUUGAAGUCAUUUAAAAAUGAGAACCAGAGUUUACAAAUACGGUCUUGUUCCUAUC |
| | GGCUAUCCACCACAGGAAGCUGUAGAUGAACUU (SEQ ID NO: 131) |
| 5 | CUCUGGAACAAACUUGUUGAGAUUUCAAGAAAAGGAACGUUGAAUAAAAAUCUGGGAA |
| | GCGAAAAAAACUGGGUCUGCCCUGCCUGUGGGU (SEQ ID NO: 132) |
| | UGAUACGGGAAAUUCUGUUAGUUUAAUAUGAAAUUGUGCCUUUGGGUAUAAAUCCUGC |
| | GCGGCCGGUUUCUUUACC (SEQ ID NO: 133) |
| | CUGGGAAGCGAAAAAAACUGGGUCUGCCCUGCCUGUGGGU (SEQ ID NO: 134) |
| | AUUUGAGUUUGCGGAAUAGUUUAAGCGUUCUCCACGGGGAUGCCUCUUUUGGCACUGA |
| | AAACCCUUGCUGUGUAAGCAGUAAGGCUGAUCCAUUGGGGAGAACUGCCAUGGCUGUU |
| | GAGGAAAGAAC (SEQ ID NO: 135) |
| | AUUUCCCGUAUCAGGAAAGGUCGUUCAGUUCUCACGUUUGCACUGGAAC |
| | (SEQ ID NO: 136) |
| 6 | UCCUGAAGCGAAUGUACAAACUUUAAGGCUGGGCUCUAGUGGCUCAGCCUCUUUU |
| | (SEQ ID NO: 137) |
| | AUAUAGCUAAAAGUGGCCCAAACAGCGCUAUCAUCCUGGCUCAGGUUAACGACGAAGA |
| | GCGUUUAACUGAAC (SEQ ID NO: 138) |
| | UUACGGCGUAAAGAGAGUAACAUUUCUUUUCCUGAAGCGAAUGUACAAACUUUAAGGC |
| | UGGGCUCUAGUGGCUCAGCCUCUUUU (SEQ ID NO: 139) |
| 7 | AUAAAUACGGACUAAUCCCUGUUGGGUAUCCGCCUGAAGAAACCAUUUCCGAGCUAUG |
| | GCGCGCCAAUAAUCUUUGGAACACGUUAGUUGGCCUCCACUAUAAAUGU (SEQ ID |
| | NO: 140) |
| | AUAAGGGGAGGAUCAGAUGACCAUUUGGCUAACUCUCCUGCCCCGCGACCAAAUAAUU |
| | AGACACGCAGAAAGCCAGCAUUUUGGUUAGUCGACAAACAGAUAAAGAUAAAACAGAG |
| | GUCCGUGUUUAUAAAUACGGACUAAUCCCUGU (SEQ ID NO: 141) |
| 8 | AUGACAAAAGAAAUUAAGGGAAUAAAAAAUGAUCAAUUGCUAUAAAUUCGGAUGCUUG |
| | CAGCC (SEQ ID NO: 142) |
| | AUUAAGGGAAUAAAAAAUGAUCAAUUGCUAUAAAUUCGGAUGCUUGCAGCC |
| | (SEQ ID NO: 143) |
| 9 | GUUGGGGGCGGAACCAAACUGGACUUGUCCAAGUUGUAAAGCAGAGCAUGACAGGGAU |
| | CACAAUGCAGCAAUAAAUAUUGCAAGG (SEQ ID NO: 144) |
| 11 | AUUUAGACGAGUCGAUGCGCAUGUGUUUUGUGUUACUCAAAAGACCCUGUAC |
| | (SEQ ID NO: 145) |
| | UGCAGUAAUUGUCCUUCUGUGUGGUGUUAAAUAAAUAUGGCUACUAGAGCAUACAAAU |
| | ACGGCUUAAUUCCUAUCGGUUAUCCCCCAAAGGAAACCAUUGAUGAGCUUUUUAAGGC |
| | AAACGUAUUAUGGAAUAACCUAGUAGCCUUACA (SEQ ID NO: 146) |
| | AUAUUUGCAGUAAUUGUCCUUCUGUGUGGUGUUAAAUAAAUAUGGCUACUAGAGCAUA |
| | CAAAUACGGCUUAAUUCCUAUCGGUUAUCCCCCAAAGGAAACCAUUGAUGAGCUUUUU |
| | AAGGCAAACGUAUUAUGGAAUAACCUAGUAGCCUUACAC (SEQ ID NO: 147) |
| 12 | AGGCUGUAGAAUCGUAGUAUAGAGUGGAGGCUCGUCUGAUUCAGAAUUACGGUGACGU |
| | AUUUUCGUUAGGCUGCCGGAGCCAGGCGUUUGGCAUCGAGCUUGGCGAGGAGAGCCUC |
| | UAAAUC (SEQ ID NO: 148) |
| 14 | AUUUCCCGUAUCAGGAAAGGUCGUUCAGUUCUCACGUUUGCACUGGAAC |
| | (SEQ ID NO: 149) |
| 15 | UCAGCUGUAGCGUAAAGAGAGUAACAUUUCUUCUCCUGACUUACUGUACAAACUUUAA |
| | GGCUGGGCUGUAAUGGCUCAGCCUCUUUU (SEQ ID NO: 150) |
| | UCCUGACUUACUGUACAAACUUUAAGGCUGGGCUGUAAUGGCUCAGCCUCUUUU |
| | (SEQ ID NO: 151) |
| 16 | AAGGGAAAAGGUGGUUUGAAAUUCCUAUACGUCCGGGGUUCUCCUCAUGUCAUCGGAG |
| | GAGCGAAUUGAUC (SEQ ID NO: 152) |
| 17 | UCAGCUGUAGCGUAAAGAGAGUAACAUUUCUUCUCCUGACUUACUGUACAAACUUUAA |
| | GGCUGGGCUGUAAUGGCUCAGCCUCUUUU (SEQ ID NO: 153) |
| | AUUGUGAGAGAAGAAAGUGAUCCACCUGCUCAGCUGUAGCGUAAAGAGAGUAACAUUU |
| | CUUCUCCUGACUUACUGUACAAACUUUAAGGCUGGGCUGUAAUGGCUCAGCCUCUUUU |
| | (SEQ ID NO: 154) |
| 18 | GUAUCUUGAGAAAAGCUGGGGAAUGCCAGAACGCAUUUUU (SEQ ID NO: 155) |
| | AGGCUCCGUGAACGGGCUUGCGGGAGCUUAGAAAACUCCGGAUGUAAGCCGCAAUUUG |
| | UUUUGCGUAUGUGGCUUGAGUUAGCGCUCAAGCCUUUUUUA (SEQ ID NO: 156) |
| 19 | GACCAAGACCAGAACGCAGCUAAAAAUUUCCUGCGAGGUCAGGAGUUUUAUCUUGCCG |
| | AAC (SEQ ID NO: 157) |
| | GACCAAGACCAGAACGCAGCUAAAAAUUUCCUGCGAGGUCAGGAGUUUUAUCUUGCCG |
| | AACAGAU (SEQ ID NO: 158) |
| | ACCGGCAUGGACAAGAGAGACAUUCAUUUCCGCUGCGAGAAAUGCGAUACUCUGCACG |
| | ACC (SEQ ID NO: 159) |
| | CUCUGGCUUCGACCAGGCUGCGCUGGACGUGCUGUACC (SEQ ID NO: 160) |
| 20 | CCAAUCUUAUGACCAGUACUUAAAAGACCCUGUAC (SEQ ID NO: 161) |
| | AUAAUUGAAAGAACUUCACCCAUUGUUUCUUCUACCAUUGAGUGACCAG (SEQ ID |
| | NO: 162) |
| | AUAAUUGAAAGAACUUCACCCAUUGUUUCUUCUACCAUUGAGU (SEQ ID NO: |
| | 163) |
| 21 | UGCCGGAUGAAGCGAAAGAUGAGCUUUUAAGGGCGAACAAUCUCUGGAACAAACUUGU |
| | UGAGAUUUCAAGAAAAGGAACGU (SEQ ID NO: 164) |
| | AUACGGGCUGGUGCCGAAGGGGUGCCUGCCGGAUGAAGCGAAAGAUGAGCUUUUAAGG |
| | GCGAACAAUCUCUGGAACAAACUUGUUGAGAUUUCAAGAAAAGGAACGU (SEQ ID |
| | NO: 165) |
| 22 | GCAAUAUAGCAAUUAAAUAAAGAUAGCUGUUAAACCAGAGCCUUAUAGAUUGCACCUC |
| | UUGAACAGUU (SEQ ID NO: 166) |
| 23 | GAUCCAACUGCUCAUUUGCAGCGUAAAGAGAGUAACAUUUCUUCUCCUGACUUACUGU |
| | AAAAACUUAAGGCUGGGCUGUAAUGGCUCAGCCUCAUU (SEQ ID NO: 167) |
| | ACUGCUCAUUUGCAGCGUAAAGAGAGUAACAUUUCUUCUCCUGACUUACUGUAAAAAC |
| | UUAAGGCUGGGCUGUAAUGGCUCAGCCUCAUU (SEQ ID NO: 168) |
| | GCUGCAAAUGAGCAGUUGGAUCACUUUGAGUUUAACGUGGUCUGUAGCCCGAU |
| | (SEQ ID NO: 169) |
| 26 | GCAAUAUAGCAAUUAAAUAAAGAUAGCUGUUAAACCAGAGCCUUAUAGAUUGCACCUC |
| | UUGAACAGUU (SEQ ID NO: 170) |
| 27 | GUGUGAAAUAAAUAUGGCUACUAGAGUAUAUAAAUAUGGCUUAAUUCCUAUCGGUUAU |
| | CCUCCAAAGGAAACCAUUCAAGAGCUAUUUAGAGCAAACGUAUUAUGGAAUAACCUAG |
| | UAGCCUUGCACAAAAAAAACAGGGAAGAUUGGGACGAUGCUCGAAGAAACAAAGACCU |
| | U (SEQ ID NO: 171) |
| | GAUGCUCGAAGAAACAAAGACCUUAGAUCUGAAAAACAAUGGUCAUGCCCCGCAUG |
| | (SEQ ID NO: 172) |
| 28 | AUAAAAGCAAUGUAAAUACUAUUGAUCAGCUAAGUAGUUCCACAACUGUUAAGAUCCU |
| | GUUUUUUAGAGUUUUGCUUAGAUUUGAUCAUGGUCUAUUUGCCAUACAAUGUUUGGUC |
| | GUUUAACCCU (SEQ ID NO: 173) |
| | AUAUAAAAGCAAUGUAAAUACUAUUGAUCAGCUAAGUAGUUCCACAACUGUUAAGAUC |
| | CUGUUUUUUAGAGUUUUGCUUAGAUUUGAUCAUGGUCUAUUUGCCAUACAAUGUUUGG |
| | UCGUUUAACCCU (SEQ ID NO: 174) |
| | AACAAAGGCCUUAGAUCUGAAAAACAAUGGUCAUGCCCUGCAUGUGGAGUUG (SEQ |
| | ID NO: 175) |
| 29 | UGUCCCUGCUGGUUGGGUAAAAACCAGACUGAUUGAGUUGAUGAUU (SEQ ID |
| | NO: 176) |
| | UAUAAAUUUACGCCCAAGCAGGGAUUUAUUUUGAGGAUUUUUAAGAACAAAGCAUUUG |
| | CGCGUUUUGC (SEQ ID NO: 177) |
| 30 | AAAGAGUGGUCAUGUCCAAACUGUGGUGUGGUAC (SEQ ID NO: 178) |
| | CAAACUGUGGUGUGGUACAUGAUAGGGAUACC (SEQ ID NO: 179) |
| 31 | GUAUCUUGAGAAAAGCUGGGGAAUGCCAGAACGCAUUUUU (SEQ ID NO: 180) |
| | AGGCUCCGUGAACGGGCUUGCGGGAGCUUAGAAAACUCCGGAUGUAAGCCGCAAUUUG |
| | UUUUGCGUAUGUGGCUUGAGUUAGCGCUCAAGCCUUUUUU (SEQ ID NO: 181) |
| | AGGCUCCGUGAACGGGCUUGCGGGAGCUUAGAAAACUCCGGAUGUAAGCCGCAAUUUG |
| | UUUUGCGUAUGUGGCUUGAGUUAGCGCUCAAGCCUUUUU (SEQ ID NO: 182) |
| 32 | GCAAUAUAGCAAUUAAAUAAAGAUAGCUGUUAAACCAGAGCCUUAUAGAUUGCACCUC |
| | UUGAACAGUU (SEQ ID NO: 183) |
| | ACUUGAUGAGUCGAUGCGCAUGUGUUUUGUGUUACUCAAAAGACCCUGUAC (SEQ |
| | ID NO: 184) |
| | UUUGAAAAAAGAGACAUCUGAGGUUAUAGAAUGAUAAAUACGAUCCUAUAUUCUCCAA |
| | CUCAAAAAGCGACUUUAUCUAAUGAUUACAAUAAGCUAUGCUCACAUCACUUGAUGAG |
| | UCGAUGCGCAUGUGUUUUGUGUUACUCAAAAGACCCUGUAC (SEQ ID NO: 185) |
| 33 | UAACGAUUUCUCUGGCACGUAUACCUAGCCGUUUACGUAACAAUAGAGUUGCCUUGAU |
| | CAUUAAGUUACUAUAUCUCUGGAG (SEQ ID NO: 186) |
| | AACGAUUUCUCUGGCACGUAUACCUAGCCGUUUACGUAACAAUAGAGUUGCCUUGAUC |
| | AUUAAGUUACUAUAUCUCUGGAG (SEQ ID NO: 187) |
| | CUCACUAGCUAAUCAUUUGUGGUUGGUCUGUUUGGUCGUGCCUUUGUUGACUGCUGCU |
| | AUUGCUCACAAACA (SEQ ID NO: 188) |
| 34 | UCCUGAAGCGAAUGUACAAACUUUAAGGCUGGGCUCUAGUGGCUCAGCCUCUUUU |
| | (SEQ ID NO: 189) |
| | UUACGGCGUAAAUAGAGUAACAUUUCUUUUCCUGAAGCGAAUGUACAAACUUUAAGGC |
| | UGGGCUCUAGUGGCUCAGCCUCUUUU (SEQ ID NO: 190) |
| 35 | CUGGGCGCCUCCCUUCCUCAUGAAGGGGGUACAACGACCCUGCGAGGUGGGUU |
| | (SEQ ID NO: 191) |
| | AUACGGGCUGGUGCCGAAGGGGUGCCUGCCGGAUGAAGCGAAAGAUGAGCUUUUAAGG |
| | GCGAACAAUCUCUGGAACAAACUUGUUGAGAUUUCAAGAAAAGGAACGU (SEQ ID |
| | NO: 192) |
| | AAAUUUUCUUUCGCCGGGGCUGGUAAAUCUCUUGCC (SEQ ID NO: 193) |
| 36 | UUUUAGCUGUCCGCUCUGGAAAACCUCGUGUGUGUAACUGUACAUGUACACAUAUCCC |
| | CUCAAAGCCAAGGCCCUCGCCUGUAC (SEQ ID NO: 194) |
| | ACUAAAAGGAAUGCUUUGAGGUAAUAGCCAAG (SEQ ID NO: 195) |
| | AUGUUUGCAAACAUGUACAGCACGUUAGCAACAAAAAGAGAGCGGGCAAUAAAGCUCA |
| | CAACGCAACUCUAUCUGCUUGUUUAGCUCAGCAAGGCU (SEQ ID NO: 196) |
| | AUGGCAACACGAGUUUACAAAUACGGUCUUGUGCCGCUUGGCUACCCACCUGAUUCAG |
| | UGGUCGGAAAAGGU (SEQ ID NO: 197) |
| | UGCCCGCUCUCUUUUUGUUGCUAACGUGCUGUACAUG (SEQ ID NO: 198) |
| | AUGGCAACACGAGUUUACAAAUACGGUCUUGUGCCGCUUGGCUACCCACCUGAUUCAG |
| | UGGUCGGAAAAGGUGGUGAGUUACAAAGAGCUAAUAAUCUCUGGAAU (SEQ ID |
| | NO: 199) |
| 37 | AUCCUCUACGGGGUGUAGUAAUCGCCAAUAUUGGGCGAGUCC (SEQ ID NO: 200) |

Unless otherwise noted, all compositions and nucleases provided herein are made in reference to the active level of that composition or nuclease, and are exclusive of impurities, for example, residual solvents or by-products, which may be present in commercially available sources. Nuclease component weights are based on total active protein. All percentages and ratios are calculated by weight unless otherwise indicated. All percentages and ratios are calculated based on the total composition unless otherwise indicated. In the exemplified composition, the nuclease levels are expressed by pure enzyme by weight of the total composition and unless otherwise specified, the ingredients are expressed by weight of the total compositions.

### Modifications

The RNA guide sequence or any of the nucleic acid sequences encoding a nuclease may include one or more covalent modifications with respect to a reference sequence, in particular the parent polyribonucleotide, which are included within the scope of this invention.

Exemplary modifications can include any modification to the sugar, the nucleobase, the internucleoside linkage (e.g. to a linking phosphate/to a phosphodiester linkage/to the phosphodiester backbone), and any combination thereof. Some of the exemplary modifications provided herein are described in detail below.

The RNA guide sequence or any of the nucleic acid sequences encoding components of a nuclease may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′ . Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine , N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1- methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6- glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylgu anosine, N1,N2-dimethylgu anosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

### Vectors

The present invention provides a vector for expressing a nuclease described herein or nucleic acids encoding a nuclease described herein may be incorporated into a vector. In some embodiments, a vector of the invention includes a nucleotide sequence encoding a nuclease described herein. In some embodiments, a vector of the invention includes a nucleotide sequence encoding a nuclease described herein.

The present invention also provides a vector that may be used for preparation of a nuclease described herein or compositions comprising a nuclease described herein. In some embodiments, the invention includes the composition or vector described herein in a cell. In some embodiments, the invention includes a method of expressing the composition comprising a nuclease of the present invention, or vector or nucleic acid encoding the nuclease, in a cell. The method may comprise the steps of providing the composition, e.g., vector or nucleic acid, and delivering the composition to the cell.

Expression of natural or synthetic polynucleotides is typically achieved by operably linking a polynucleotide encoding the gene of interest, e.g., nucleotide sequence encoding a nuclease of the present invention, to a promoter and incorporating the construct into an expression vector. The expression vector is not particularly limited as long as it includes a polynucleotide encoding a nuclease of the present invention and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses which are useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of a nuclease of the present invention from a polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding a nuclease of the present invention has been transferred into the host cells and then expressed without fail.

The preparation method for recombinant expression vectors is not particularly limited, and examples thereof include methods using a plasmid, a phage or a cosmid.

### Cells

The nucleases described herein can be introduced into a variety of cells. In some embodiments, the cell is an isolated cell. In some embodiments the cell is in cell culture. In some embodiments, the cell is ex vivo. In some embodiments, the cell is obtained from a living organism, and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, 293T, MF7, K562, HeLa, and transgenic varieties thereof. Cell lines are available from a variety of sources known to those with skill in the art (see, e.g., the American Type Culture Collection (ATCC) (Manassas, Va.)). In some embodiments, a cell transfected with one or more nucleic acids (such as nuclease polypeptide encoding vector and RNA guide) is used to establish a new cell line comprising one or more vector-derived sequences to establish a new cell line comprising modification to the target nucleic acid or target locus. In some embodiments, the cell is an immortal or immortalized cell.

In some embodiments, the cell is a primary cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a mammalian cell, e.g., a human cell or a murine cell. In some embodiments, the murine cell is derived from a wild-type mouse, an immunosuppressed mouse, or a disease-specific mouse model.

### Production

In some embodiments, a nuclease of the present invention can be prepared by (I) culturing bacteria which produce a nuclease of the present invention, isolating the nuclease, and optionally, purifying the nuclease. The nuclease can be also prepared by (II) a known genetic engineering technique, specifically, by isolating a gene encoding a nuclease of the present invention from bacteria, constructing a recombinant expression vector, and then transferring the vector into an appropriate host cell for expression of a recombinant protein. Alternatively, a nuclease can be prepared by (III) an in vitro coupled transcription-translation system. Bacteria that can be used for preparation of a nuclease of the present invention are not particularly limited as long as they can produce a nuclease of the present invention. Some non-limiting examples of the bacteria include E. coli cells described herein.

### Methods of Expression

The present invention includes a method for protein expression, comprising translating a nuclease described herein.

In some embodiments, a host cell described herein is used to express a nuclease. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production of a nuclease. After expression of the nuclease, the host cells can be collected and nuclease purified from the cultures etc. according to conventional methods (for example, filtration, centrifugation, cell disruption, gel filtration chromatography, ion exchange chromatography, etc.).

In some embodiments, the methods for nuclease expression comprises translation of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids of a nuclease. In some embodiments, the methods for protein expression comprises translation of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, about 1000 amino acids or more of a nuclease.

A variety of methods can be used to determine the level of production of a mature nuclease in a host cell. Such methods include, but are not limited to, for example, methods that utilize either polyclonal or monoclonal antibodies specific for a nuclease. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of a nuclease in a cell, comprising providing a polyribonucleotide encoding the nuclease to a host cell wherein the polyribonucleotide encodes the nuclease, expressing the nuclease in the cell, and obtaining the nuclease from the cell.

### Delivery

Compositions described herein may be formulated, for example, including a carrier, such as a carrier and/or a polymeric carrier, e.g., a liposome, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof. In another aspect, the disclosure is directed, in part, to an AAV particle comprising an AAV vector described herein. In some embodiments, the AAV particle is an AAV1, AAV2, AAV3, AAV4, AAV5, AAV6, AAV7, AAV8, AAV9, AAV10, or AAV11 particle (e.g., an AAV8, AAV3, or AAV2 particle). In some embodiments, the AAV particle comprises an AAV capsid. In some embodiments, the AAV capsid comprises one or more AAV1, AAV2, AAV3, AAV4, AAV5, AAV6, AAV7, AAV8, AAV9, AAV10, or AAV11 proteins. In some embodiments, all the protein components of the AAV capsid are proteins of the same AAV serotype (e.g., all AAV1, AAV2, AAV3, AAV4, AAV5, AAV6, AAV7, AAV8, AAV9, AAV10, or AAV11 proteins).

In some embodiments, the method comprises delivering one or more nucleic acids (e.g., nucleic acids encoding a nuclease, RNA guide, donor DNA, etc.), one or more transcripts thereof, and/or a pre-formed nuclease/RNA guide complex to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnectofection or magnet assisted transfection, particle bombardment; and hybrid methods, such as nucleofection. In some embodiments, the present application further provides cells produced by such methods, and organisms (such as animals, plants, or fungi) comprising or produced from such cells.

All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Analysis of Effector Sequences

In this Example, amino acid sequences of the sequences of SEQ ID NOs: 1-37 and SEQ ID NOs: 221-224 were analyzed to identify potential functional protein domains. The amino acid sequences were determined to include a putative C-terminal RuvC domain The catalytic residues were also determined to reside in conserved sequence motifs (I, II, and III) of the RuvC domain The predicted catalytic residues and RuvC domain ranges of three representative effectors are shown in Table 7.

TABLE 7
RuvC Domain Features.

| Effector | RuvC Catalytic Residues | RuvC Domain Range (by amino acid position) |
|---|---|---|
| SEQ ID NO: 2 | D280, E439, D560 | 253-570 |
| SEQ ID NO: 19 | D288, E454, D575 | 261-585 |
| SEQ ID NO: 33 | D278, E437, D558 | 251-568 |

The amino acid sequences of SEQ ID NOs: 1-37 and SEQ ID NOs: 221-224 were further aligned to identify regions of sequence similarity, as shown in FIGS. 1A-1L. The consensus sequence is set forth at the top of FIGS. 1A-1L. Below the consensus sequence, a bar graph depicts sequence similarity, with the tallest bars indicating the residues with the highest sequence similarity. Non-limiting regions of sequence similarity are shown in Table 8.

TABLE 8
Conserved Sequences.

| Sequence | Residues | Position |
|---|---|---|
| X₁X₂X₃X₄GX₅X₆ | X₁ is V or A or C | N-terminal |
| (SEQ ID NO: 233) | X₂ is Y or F | |
| | X₃ is K or Q | |
| | X₄ is Y or F | |
| | X₅ is L or A or M or C or T | |
| | X₆ is I or V or L | |
| LX₁NX₂LV | X₁ is W or K or R | N-terminal |
| (SEQ ID NO: 234) | X₂ is N or T or K or S or D or Q | |
| FDX₁X₂G | X₁ is G or Y | N-terminal |
| (SEQ ID NO: 235) | X₂ is T or S or M | |
| X₁X₂HR X₃X₄P | X₁ is I or L or V | Mid sequence |
| (SEQ ID NO: 236) | X₂ is Y or L or M or F | |
| | X₃ is P or H or D or E | |
| | X₄ is L or I or V or M | |
| GX₁DX₂GX₃R | X₁ is I or L or V X₂ is I or V or L | Mid sequence |
| (SEQ ID NO: 237) | X₃ is F or Y | |
| RX₁X₂X₃YR | X₁ is K or Q or E | C-terminal |
| (SEQ ID NO: 238) | X₂ is H or D or E | |
| | X₃ is F or V or L or I | |
| X₁DX₂DX₃NAAX₄N | X₁ is H or Y | C-terminal |
| (SEQ ID NO: 239) | X₂ is R or Q or V | |
| | X₃ is E or T or I or H or K or Q or D | |
| | X₄ is N or R or I or Vor K | |

This Example indicates that the effectors of SEQ ID NOs: 1-37 and SEQ ID NOs: 221-224 were classified as a family with a conserved C-terminal RuvC domain representative of nucleases.

### Example 2—Expression of Effectors in E. coli

In this Example, a system individually comprising an effector of any one of SEQ ID NOs: 2, 5-7, 11, 13-21, 23, 27, 28, 31, and 34-37 was engineered and introduced into E. coli.

For each effector, a polynucleotide encoding the effector was E. coli codon-optimized, synthesized (Genscript), and individually cloned into a custom expression system derived from pET-28a(+) (EMD-Millipore). The vector included a polynucleotide encoding each effector under the control of a lac promoter and an E. coli ribosome binding sequence. The vector also included a site for a pre-crRNA (direct repeat-spacer-direct repeat) driven by a J23119 promoter following the open reading frame for the effector. For each effector, the direct repeat sequences tested are set forth in Table 4. The spacers were designed to target sequences of a pACYC184 plasmid and E. coli essential genes.

The effector/pre-crRNA plasmids were electroporated into E. Cloni electrocompetent E. coli (Lucigen). The effector/pre-crRNA plasmids were either co-transformed with purified pACYC184 plasmid or directly transformed into pACYC184-containing E. Cloni electrocompetent E. coli (Lucigen), plated onto agar containing the proper antibiotics, and incubated for 10-12 hours at 37° C.

A proxy for activity of the engineered effector/pre-crRNA system in E. coli was investigated, wherein bacterial cell death was used as the proxy for system activity. An active effector associated with a pre-crRNA could disrupt expression of a spacer sequence target, e.g., a pACYC184 plasmid sequence or an E. coli essential gene, resulting in cell death. Using this proxy, the effectors disclosed herein were determined to have activity in E. coli.

Thus, this Example suggests that the effectors of SEQ ID NOs: 2, 5-7, 10, 11, 13-21, 23, 27, 28, 31, and 34-37 were capable of being expressed in bacterial cells. With a pre-crRNA (direct repeat-spacer-direct repeat), the effectors of SEQ ID NOs: 2, 5-7, 10, 11, 13-21, 23, 27, 28, 31, and 34-37 were shown to have activity in bacterial cells.

### Example 3—Purification of Effector of SEQ ID NO: 2

This Example describes the expression and purification of the effector of SEQ ID NO: 2.

NEB NiCo21(DE3) cells were transformed with an expression plasmid comprising a polynucleotide encoding a His-tagged version of the effector of SEQ ID NO: 2. The cells were grown at 37° C. until reaching an OD₆₀₀ of 0.6-1.0. IPTG was added to the culture for a final concentration of 0.2 mM, and the cell growth continued at 16° C. for 12-16 hours. Cells were then pelleted and resuspended in 100 mL of Buffer A (50 mM HEPES KOH pH 7.8, 500 mM NaCl, 10 mM MgCl₂, 20 mM imidazole, 14 mM β-mercaptoethanol, and 5% glycerol). The resuspended cells were mixed 30-45 minutes at 4° C. and subsequently lysed using a high-pressure cell disrupter. The lysed cells were centrifuged at 45,000×g and 4° C. for 30 minutes, and the supernatant was transferred to fresh tubes and centrifuged at 45,000×g and 4° C. for 30 minutes.

The supernatant was then applied to a HisTrap Nickel 5 mL column, and the eluted protein fractions were analyzed by SDS-PAGE. The fractions comprising the purified effector protein were then combined, dialyzed using a 10 kDa MWCO, and concentrated. The final protein concentration was measured using a Bradford assay.

This Example thus demonstrates that the effector of SEQ ID NO: 2 was able to be expressed and purified for nuclease activity assays, which are described in the next Examples.

### Example 4—Double-stranded DNA Cleavage by Effector of SEQ ID NO: 2

This Example demonstrates double-stranded DNA (dsDNA) cleavage by the effector of SEQ ID NO: 2.

An RNA guide for the effector of SEQ ID NO: 2 was prepared using in vitro transcription (IVT). The spacer sequence of the RNA guide of SEQ ID NO: 202, set forth in SEQ ID NO: 203, was designed to have complementary to Target A (SEQ ID NO: 201). Target B (SEQ ID NO: 204) had no complementarity to the spacer sequence (SEQ ID NO: 203) and was thus used as a non-target control. The bolded portion of Target A (SEQ ID NO: 201) in Table 9 corresponds to the sequence to which the mature crRNA of SEQ ID NO: 202 binds. dsDNA templates for the IVT reaction were prepared using a second strand fill in method. The oligo template containing a T7 promoter sequence was synthesized commercially (IDT) and annealed to a reverse primer followed by extension to fill in the second strand (Klenow polymerase, large fragment, NEB). IVT was performed by incubating the dsDNA templates with T7 RNA polymerase (HiScribe T7 Quick High Yield RNA synthesis kit NEB) followed by treatment with DNase (Thermo Fisher Scientific) to remove the DNA template. The IVT product was cleaned up using an RNA prep kit (Zymo Research).

TABLE 9
Sequences for DNA cleavage assay.

| | | mature crRNA | |
|---|---|---|---|
| Target | Target Sequence | Sequend | Spacer Sequence |
| A | TCCATGTCTCGTTATACGCTGTGGTTCGCCA | AUAACGACCCUGCG | GGAUCGCUUCGCCA |
| | ACATCACACGCACTACTTTTGGCAGACGC [symbol] | AAGUGGGGUGUAAC | UUGUAGCCGUAGGU |
| | [symbol] [symbol] [symbol] | UUCGACGGAUCGCU | CU |
| | TATTCATGTACTACTTCACACGCTTGACAGC | UCGCCAUU | (SEQ ID NO: 203) |
| | TAGCTCAGTCCTAGGTATAAT | (SEQ ID NO: 202) | |
| | (SEQ ID NO: 201) | | |
| B | TCCATGTCTCGTTATACGCTGTGGTTCGCCA | | |
| | ACGCAGCTAATTCACTACTAGCGGCTTCTTT | | |
| | TTCAACGATGCCACCCGGTTGGTTCAGGGCC | | |
| | GGAACCGGCTTACTACTGCTAATTCTTGACA | | |
| | GCTAGCTCAGTCCTAGGTATAAT | | |
| | (SEQ ID NO: 204) | | |

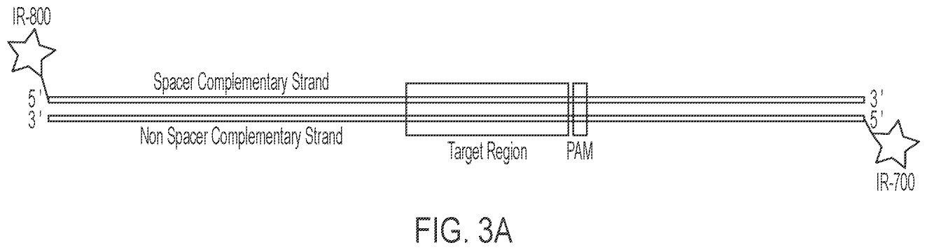

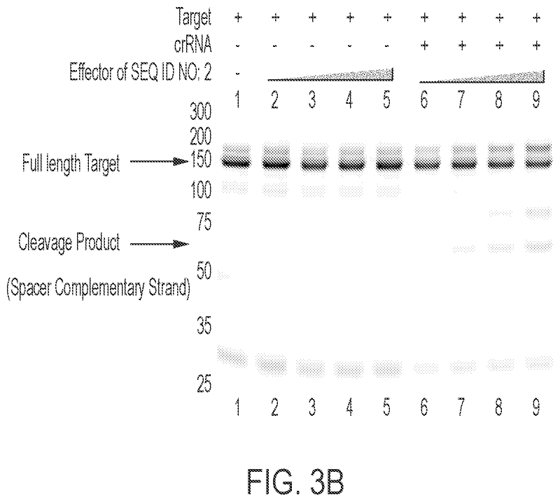

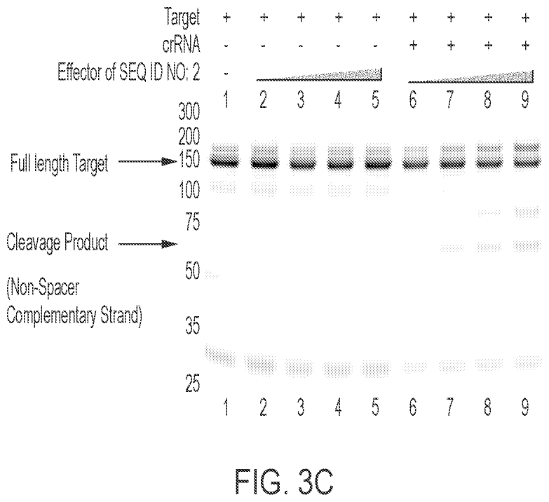

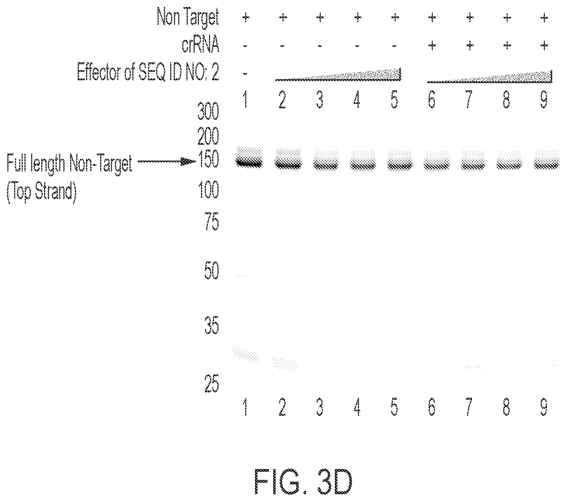

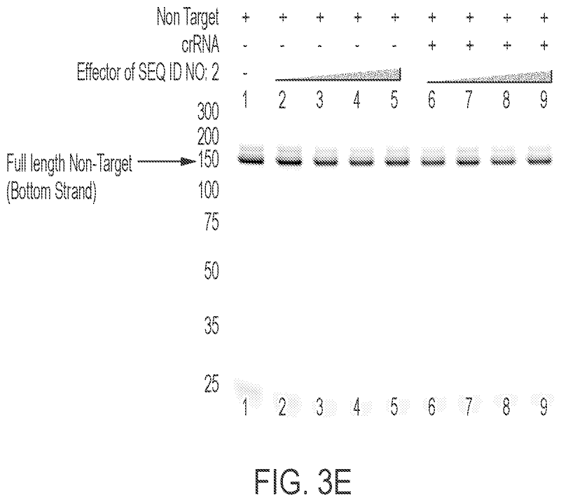

Dual-labeled dsDNA target (Target A) and non-target (Target B) substrates were generated via PCR using an IR800-labelled forward primer (IDT) and an IR700-labelled reverse primer (IDT). The resulting PCR product comprised an IR700 label on the non-spacer complementary (NSC) strand and an IR800 label on the spacer complementary strand, as shown in FIG. 3A. These substrates were purified using SPRI beads (Agilent), and concentrations were measured using nanodrop (Thermo Fisher Scientific). The IR700 and IR800 labels could be visualized in separate fluorescence channels without any cross over, thus allowing for visualization of cleavage of both strands of the target.

dsDNA target cleavage assays were set up in reaction buffer (1× NEBuffer2, NEB). Complexed RNPs (effector plus RNA guide) were formed by incubating purified effector with the RNA guide at a ratio of 1:2. Complexed RNPs were then added to 40 nM dsDNA substrate and incubated. Negative controls with no effector or no RNA guide were also tested. Reactions were treated with an RNase cocktail and incubated, followed by treatment and incubation with Proteinase K.

To detect dsDNA cleavage, DNA products from the reactions were analyzed on 15% TBE-Urea gels. Gels were imaged on a fluorescent digital imaging system (LI-COR Biosciences) for both IR800 and IR700 fluorescence.

As shown in FIG. 3B and FIG. 3C, target-specific double-stranded cleavage was observed using an RNP comprising the effector of SEQ ID NO: 2 and the RNA guide of SEQ ID NO: 202. FIG. 3B shows cleavage of the spacer complementary strand of the dsDNA target (IR800 image), and FIG. 3C shows cleavage of the non-spacer complementary strand of the dsDNA target (IR700 image). Cleavage was positively correlated with the effector concentration, as shown in lanes 6-9 of FIG. 3B and lanes 6-9 of FIG. 3C. No detectable cleavage activity was observed in the absence of the RNA guide and/or in the absence of the effector, as shown in lanes 2-5 of FIG. 3B and lanes 2-5 of FIG. 3C, respectively. Furthermore, no detectable cleavage activity was observed for the effector of SEQ ID NO: 2 complexed with a non-targeting RNA guide, as shown in FIG. 3D and FIG. 3E. For example, no detectable cleavage was observed for the Target B spacer complementary strand (FIG. 3D) or for the Target B non-spacer complementary strand (FIG. 3E) when using the RNA guide designed for Target A.

This Example thus shows that the effector of SEQ ID NO: 2 has nuclease activity and catalyzes target-specific dsDNA cleavage.

### Example 5—Single-Stranded DNA Cleavage by Effector of SEQ ID NO: 2

This Example demonstrates single-stranded DNA (ssDNA) cleavage by the effector of SEQ ID NO: 2.

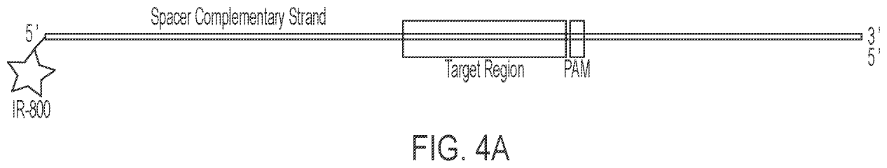

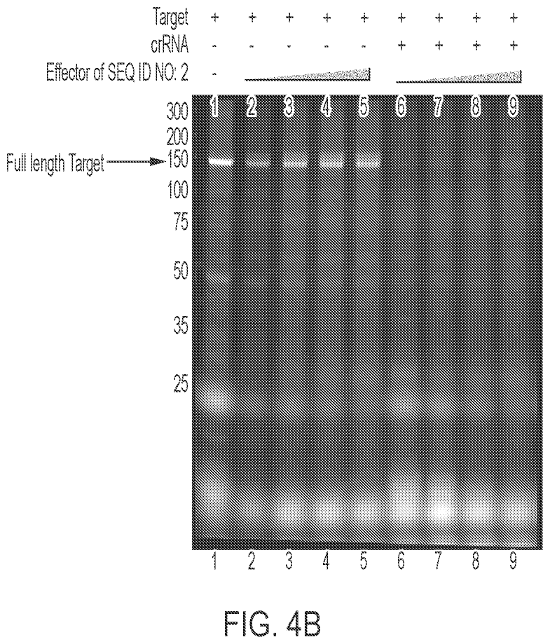

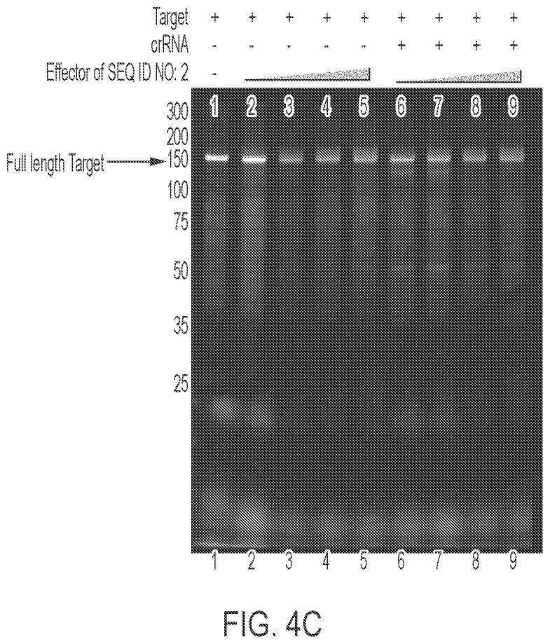

To generate labelled ssDNA target as shown in FIG. 4A, a ssDNA oligo from IDT was labelled with near-infrared fluorescent dye (IR-800) using 5′ labeling kit (Vector Labs) following the manufacturer' s protocol. ssDNA target cleavage assays were set up in reaction buffer (NEBuffer2) as described in Example 4. Negative controls with no effector or with non-target ssDNA were also tested.

An RNP complex was generated by incubating the effector of SEQ ID NO: 2 with RNA guide (SEQ ID NO: 202) at a ratio of 1:2 in the assay buffer before adding near-infrared fluorescent dye labelled ssDNA of Target A (SEQ ID NO: 201) from Example 4 (and shown in FIG. 4A) and incubating. Negative control non-target ssDNA (SEQ ID NO: 204) was incubated with an RNP comprising the effector of SEQ ID NO: 2 and the RNA guide of SEQ ID NO: 202 in a similar fashion. Reactions were first treated with an RNase cocktail and Proteinase K as described in Example 4. To detect ssDNA cleavage products, the reactions were analyzed on a 15% TBE-Urea gel and imaged as described in Example 4.

As shown in lanes 6-9 of FIG. 4B, target-specific ssDNA cleavage was observed, as evidenced by the absence of the full-length bands. No significant cleavage can be observed for non-target ssDNA, as shown in lanes 6-9 of FIG. 4C. The effector of SEQ ID NO: 2 demonstrated efficient cleavage activity against ssDNA targets such that complete cleavage was observed even at the lowest RNP concentration tested (125 nM — lane 6, FIG. 4B). As shown in the lane 2, no detectable cleavage product was observed for the non-target ssDNA, even at the highest RNP concentration tested (1 μM).

This Example thus shows that the effector of SEQ ID NO: 2 has nuclease activity and catalyzes target-specific ssDNA cleavage.

### Example 6—In vitro Targeting of GFP by Effector of SEQ ID NO: 2

This Example describes use of a fluorescence depletion assay (FDA) to measure activity of the effector of SEQ ID NO: 2.

In this assay, an active CRISPR system designed to target GFP binds and cleaves the double-stranded DNA region encoding GFP, resulting in depletion of GFP fluorescence. The FDA assay involves in vitro transcription and translation, allowing production of an RNP from a DNA template encoding a CLUST.200916 effector and a DNA template containing a pre-crRNA sequence under a T7 promoter with direct repeat (DR)-spacer-direct repeat (DR); the spacer targeted GFP. In the same one-pot reaction, GFP and RFP were also produced as both the target and the fluorescence reporter (FIG. SA). The target GFP plasmid sequence is set forth in SEQ ID NO: 205, and the fluorescence reporter RFP plasmid sequence is set forth in SEQ ID NO: 206. GFP and RFP fluorescence values were measured every 20 min at 37° C. for 12 hr, using a TECAN Infinite F Plex plate reader. Since RFP was not targeted, its fluorescence was not affected and was therefore used as an internal signal control.

SEQ ID NO: 205:
ccccttgtattactgtttatgtaagcagacaggatgcgtccggcgtagaggatcgagatctcCAAAAAAT
GGCTGTTTTTGAAAAAAATTCTAAAGGTTGTTTTACGACAGACGATAACAGGGTTgaaataattttgttt
aactttaagaaggagATTTAAATatgAAAATCGAAGAAGGTAAAGGTCACCATCACCATCACCACggatc
catgacggcattgacggaaggtgcaaaactgtttgagaaagagatcccgtatatcaccgaactggaaggc
gacgtcgaaggtatgaaatttatcattaaaggcgagggtaccggtgacgcgaccacgggtaccattaaag
cgaaatacatctgcactacgggcgacctgccggtcccgtgggcaaccctggtgagcaccctgagctacgg
tgttcagtgtttcgccaagtacccgagccacatcaaggatttctttaagagcgccatgccggaaggttat
acccaagagcgtaccatcagcttcgaaggcgacggcgtgtacaagacgcgtgctatggttacctacgaac
gcggttctatctacaatcgtgtcacgctgactggtgagaactttaagaaagacggtcacattctgcgtaa
gaacgttgcattccaatgcccgccaagcattctgtatattctgcctgacaccgttaacaatggcatccgc
gttgagttcaaccaggcgtacgatattgaaggtgtgaccgaaaaactggttaccaaatgcagccaaatga
atcgtccgttggcgggctccgcggcagtgcatatcccgcgttatcatcacattacctaccacaccaaact
gagcaaagaccgcgacgagcgccgtgatcacatgtgtctggtagaggtcgtgaaagcggttgatctggac
acgtatcagTAATAAaaagcccgaaaggaagctgagttggctgctgccaccgctgagcaataactagcat
aaccccttggggcctctaaacgggtcttgaggggttttttgctgaaaggaggaactatatccggCTTCCT
CGCTCACTGACTCGCTGCGCTCGGTCGTTCGGCTGCGGCGAGCGGTATCAGCTCACTCAAAGGCGGTAAT
ACGGTTATCCACAGAATCAGGGGATAACGCAGGAAAGAACATGTGAGCAAAAGGCCAGCAAAAGGCCAGG
AACCGTAAAAAGGCCGCGTTGCTGGCGTTTTTCCATAGGCTCCGCCCCCCTGACGAGCATCACAAAAATC
GACGCTCAAGTCAGAGGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCTCCAAGCTGGGCT
GTGTGCACGAACCCCCCGTTCAGCCCGACCGCTGCGCCTTATCCGGTAACTATCGTCTTGAGTCCAACCC
GGTAAGACACGACTTATCGCCACTGGCAGCAGCCACTGGTAACAGGATTAGCAGAGCGAGGTATGTAGGC
GGTGCTACAGAGTTCTTGAAGTGGTGGCCTAACTACGGCTACACTAGAAGAACAGTATTTGGTATCTGCG
CTCTGCTGAAGCCAGTTACCTTCGGAAAAAGAGTTGGTAGCTCTTGATCCGGCAAACAAACCACCGCTGG
TAGCGGTGGTTTTTTTGTTTGCAAGCAGCAGATTACGCGCAGAAAAAAAGGATCTCAAGAAGATCCTTTG
ATCTTTTCTACGGGGTCTGACGCTCAGTGGAACGAAAACTCACGggtggcacttttcggggaaatgtgcg
cggaacccctatttgtttatttttctaaatacattcaaatatgtatccgctcatgaattaattcttagaa
aaactcatcgagcatcaaatgaaactgcaatttattcatatcaggattatcaataccatatttttgaaaa
agccgtttctgtaatgaaggagaaaactcaccgaggcagttccataggatggcaagatcctggtatcggt
ctgcgattccgactcgtccaacatcaatacaacctattaatttcccctcgtcaaaaataaggttatcaag
tgagaaatcaccatgagtgacgactgaatccggtgagaatggcaaaagtttatgcatttctttccagact
tgttcaacaggccagccattacgctcgtcatcaaaatcactcgcatcaaccaaaccgttattcattcgtg
attgcgcctgagcgagacgaaatacgcgatcgctgttaaaaggacaattacaaacaggaatcgaatgcaa
ccggcgcaggaacactgccagcgcatcaacaatattttcacctgaatcaggatattcttctaatacctgg
aatgctgttttcccggggatcgcagtggtgagtaaccatgcatcatcaggagtacggataaaatgcttga
tggtcggaagaggcataaattccgtcagccagtttagtctgaccatctcatctgtaacatcattggcaac
gctacctttgccatgtttcagaaacaactctggcgcatcgggcttcccatacaatcgatagattgtcgca
cctgattgcccgacattatcgcgagcccatttatacccatataaatcagcatccatgttggaatttaatc
gcggcctagagcaagacgtttcccgttgaatatggctcataaca
SEQ ID NO: 206:
ccccttgtattactgtttatgtaagcagacaggatgcgtccggcgtagaggatcgagatctcCAAAAAAT
GGCTGTTTTTGAAAAAAATTCTAAAGGTTGTTTTACGACAGACGATAACAGGGTTgaaataattttgttt
aactttaagaaggagATTTAAATatgAAAATCGAAGAAGGTAAAGGTCACCATCACCATCACCACggatc
caTGGTCAGCAAGGGGGAGGAAGACAATATGGCTATTATCAAGGAATTCATGCGCTTCAAGGTGCATATG
GAAGGAAGCGTGAATGGACACGAATTCGAGATCGAAGGCGAGGGGGAGGGTCGCCCTTATGAAGGCACAC
AAACAGCTAAACTGAAAGTGACGAAGGGAGGGCCGCTTCCCTTCGCTTGGGACATTCTTTCACCCCAGTT
CATGTATGGTTCAAAGGCTTATGTCAAGCACCCGGCGGACATTCCAGACTACTTAAAATTGTCGTTCCCC
GAGGGGTTTAAATGGGAACGCGTTATGAATTTCGAGGATGGGGGAGTCGTAACGGTTACCCAGGACAGTA
GCCTGCAGGATGGCGAGTTCATCTACAAAGTGAAATTGCGCGGGACGAACTTCCCTAGCGATGGGCCAGT
CATGCAGAAGAAAACGATGGGATGGGAAGCGTCATCCGAGCGCATGTATCCTGAAGATGGTGCTTTAAAA
GGTGAGATCAAGCAGCGTTTGAAACTGAAGGACGGGGGCCATTATGATGCTGAAGTTAAAACGACATATA
AGGCCAAGAAGCCAGTTCAACTGCCAGGGGCTTATAATGTTAATATTAAATTAGACATTACGAGCCATAA
TGAAGATTACACGATTGTCGAGCAATACGAGCGCGCAGAAGGACGCCACTCAACGGGGGGCATGGACGAG
CTGTACAAGTAAaaagcccgaaaggaagctgagttggctgctgccaccgctgagcaataactagcataac
cccttggggcctctaaacgggtcttgaggggttttttgctgaaaggaggaactatatccggCTTCCTCGC
TCACTGACTCGCTGCGCTCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGACGAGCATCACAAAAATCGAC
GCTCAAGTCAGAGGTGGCGAAACCCGACAGGACTATAAAGATACCAGGCGTTTCCCCCTGGAAGCTCCCT
CGTGCGCTCTCCTGTTCCGACCCTGCCGCTTACCGGATACCTGTCCGCCTTTCTCCCTTCGGGAAGCGTG
GCGCTTTCTCATAGCTCACGCTGTAGGTATCTCAGTTCGGTGTAGGTCGTTCGCTCCAAGCTGGGCTGTG
TGCACGAACCCCCCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAGGTATGTAGGCGGT
GCTACAGAGTTCTTGAAGTGGTGGCCTAACTACGGCTACACTAGAAGAACAGTATTTGGTATCTGCGCTC
TGCTGAAGCCAGTTACCTTCGGAAAAAGAGTTGGTAGCTCTTGATCCGGCAAACAAACCACCGCTGGTAG
CGGTGGTTTTTTTGTTTGCAAGCAGCAGATTACGCGCAGAAAAAAAGGATCTCAAGAAGATCCTTTGATC
TTTTCTACGGGGTCTGACGCTCAGTGGAACGAAAACTCACGggtggcacttttcggggaaatgtgcgcgg
aacccctatttgtttatttttctaaatacattcaaatatgtatccgctcatgaattaattcttagaaaaa
ctcatcgagcatcaaatgaaactgcaatttattcatatcaggattatcaataccatatttttgaaaaagc
cgtttctgtaatgaaggagaaaactcaccgaggcagttccataggatggcaagatcctggtatcggtctg
cgattccgactcgtccaacatcaatacaacctattaatttcccctcgtcaaaaataaggttatcaagtga
gaaatcaccatgagtgacgactgaatccggtgagaatggcaaaagtttatgcatttctttccagacttgt
tcaacaggccagccattacgctcgtcatcaaaatcactcgcatcaaccaaaccgttattcattcgtgatt
gcgcctgagcgagacgaaatacgcgatcgctgttaaaaggacaattacaaacaggaatcgaatgcaaccg
gcgcaggaacactgccagcgcatcaacaatattttcacctgaatcaggatattcttctaatacctggaat
gctgttttcccggggatcgcagtggtgagtaaccatgcatcatcaggagtacggataaaatgcttgatgg
tcggaagaggcataaattccgtcagccagtttagtctgaccatctcatctgtaacatcattggcaacgct
acctttgccatgtttcagaaacaactctggcgcatcgggcttcccatacaatcgatagattgtcgcacct
gattgcccgacattatcgcgagcccatttatacccatataaatcagcatccatgttggaatttaatcgcg
gcctagagcaagacgtttcccgttgaatatggctcataaca

5 GFP targets (plus 1 non-target) were designed for screening the effector of SEQ ID NO: 2. RNA guide sequences, target sequences, and the non-target control sequences used for the FDA assay are listed in Table 10. The pre-crRNA sequences shown in Table 10 further include a T7 promoter at the 5′ end and a hairpin motif that caps the 3′ end of the RNA to ensure that the RNA is not degraded by nucleases present in the in vitro transcription and translation mixture.

TABLE 10
RNA guide and Target Sequences for FDA Assay.

| | | | PAM |
|---|---|---|---|
| Target | pre-crRNA Sequence | Target Sequence | Sequence |
| Target 1 | GAAAUUAAUACGACUCACUAUAGGUAUAACG | AGAGCGTACCATCAGCT | 5′-CCCA-3′ |
| | ACCCUGCGAAGUGGGGUGUAACUUCGACAGA | TCGAAGGCGACGG | |
| | GCGUACCAUCAGCUUCGAAGGCGACGGGUAU | (SEQ ID NO: 208) | |
| | AACGACCCUGCGAAGUGGGGUGUAACUUCGA | | |
| | CCUAACCCCUCUCUAAACGGAGGGGUUU | | |
| | (SEQ ID NO: 207) | | |
| Target 2 | GAAAUUAAUACGACUCACUAUAGGUAUAACG | AAAAACTGGTTACCAAA | 5′-ACCG-3′ |
| | ACCCUGCGAAGUGGGGUGUAACUUCGACAAA | TGCAGCCAAATGA | |
| | AACUGGUUACCAAAUGCAGCCAAAUGAGUAU | (SEQ ID NO: 210) | |
| | AACGACCCUGCGAAGUGGGGUGUAACUUCGA | | |
| | CCUAACCCCUCUCUAAACGGAGGGGUUU | | |
| | (SEQ ID NO: 209) | | |
| Target 3 | GAAAUUAAUACGACUCACUAUAGGUAUAACG | TAGTGCAGATGTATTTC | 5′-CCCG-3′ |
| | ACCCUGCGAAGUGGGGUGUAACUUCGACUAG | GCTTTAATGGTAC | |
| | UGCAGAUGUAUUUCGCUUUAAUGGUACGUAU | (SEQ ID NO: 212) | |
| | AACGACCCUGCGAAGUGGGGUGUAACUUCGA | | |
| | CCUAACCCCUCUCUAAACGGAGGGGUUU | | |
| | (SEQ ID NO: 211) | | |
| Target 4 | GAAAUUAAUACGACUCACUAUAGGUAUAACG | TGATGTGGCTCGGGTAC | 5′-TCCT-3′ |
| | ACCCUGCGAAGUGGGGUGUAACUUCGACUGA | TTGGCGAAACACT | |
| | UGUGGCUCGGGUACUUGGCGAAACACUGUAU | (SEQ ID NO: 214) | |
| | AACGACCCUGCGAAGUGGGGUGUAACUUCGA | | |
| | CCUAACCCCUCUCUAAACGGAGGGGUUU | | |
| | (SEQ ID NO: 213) | | |
| Target 5 | GAAAUUAAUACGACUCACUAUAGGUAUAACG | TGACGGCATTGACGGAA | 5′-TCCA-3′ |
| | ACCCUGCGAAGUGGGGUGUAACUUCGACUGA | GGTGCAAAACTGT | |
| | CGGCAUUGACGGAAGGUGCAAAACUGUGUAU | (SEQ ID NO: 216) | |
| | AACGACCCUGCGAAGUGGGGUGUAACUUCGA | | |
| | CCUAACCCCUCUCUAAACGGAGGGGUUU | | |
| | (SEQ ID NO: 215) | | |
| Non- | GAAAUUAAUACGACUCACUAUAGGUAUAACG | | |
| Target | ACCCUGCGAAGUGGGGUGUAACUUCGACGAG | | |
| Control | CGAGACGAAAUACGCGAUCGCUGUUAAGUAU | | |
| | AACGACCCUGCGAAGUGGGGUGUAACUUCGA | | |
| | CCUAACCCCUCUCUAAACGGAGGGGUUU | | |
| | (SEQ ID NO: 217) | | |

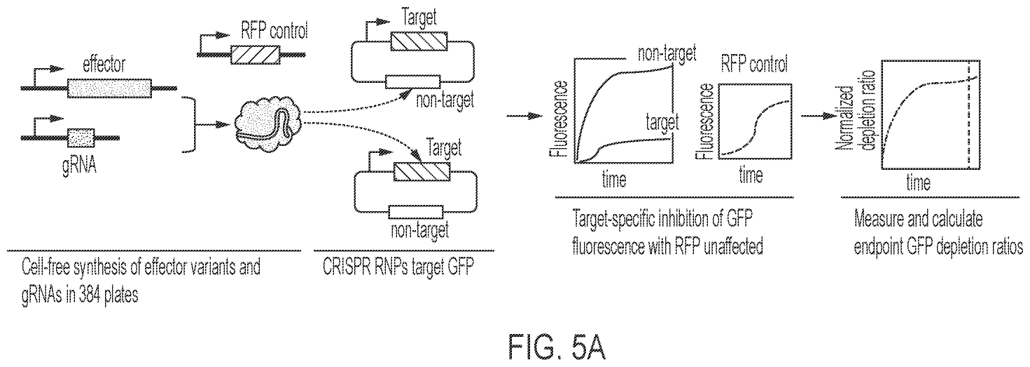

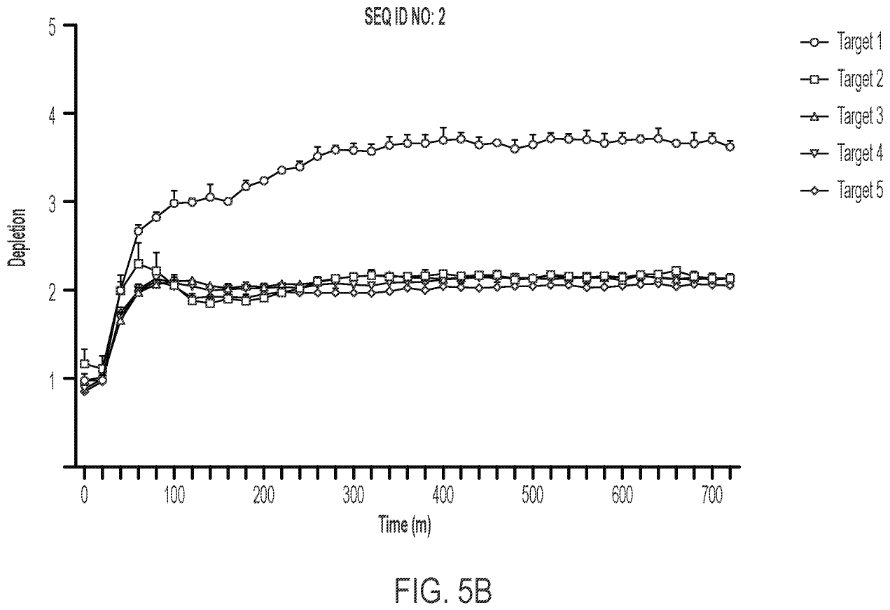

GFP signal was normalized to RFP signal, then the average fluorescence of three technical replicates was taken at each time point. GFP fluorescence depletion was then calculated by dividing the GFP signal of an effector incubated with a non-GFP targeting RNA guide (which instead targets a kanamycin resistance gene) by the GFP signal of an effector incubated with a GFP targeting RNA guide. The resulting value is referred to as “Depletion” in FIG. 5B.

A Depletion of one or approximately one indicated that there was little to no difference in GFP depletion with respect to a non-GFP targeting pre-crRNA and a GFP targeting pre-crRNA (e.g., 10 RFU/10 RFU =1). A Depletion of greater than one indicated that there was a difference in GFP depletion with respect to a non-GFP targeting pre-crRNA and a GFP targeting pre-crRNA (e.g., 10 RFU/5 RFU =2). Depletion of the GFP signal indicated that the effector formed a functional RNP and interfered with the production of GFP by introducing double-stranded DNA cleavage within the GFP coding region. The extent of the GFP depletion was largely correlated to the specific activity of the effector of SEQ ID NO: 2.

FIG. 5B shows depletion curves for RNPs formed by the effector of SEQ ID NO: 2, using values measured every 20 minutes for each of the GFP targets. At each target, the depletion values for the RNP formed with the effector of SEQ ID NO: 2 were greater than 1.

This indicated that the effector of SEQ ID NO: 2 formed a functional RNP capable of interfering with the production of GFP.

### Example 7—Targeting of Mammalian Gene by Effector of SEQ ID NO: 2

This Example describes an indel assessment on a mammalian AAVS1 target by the effector of SEQ ID NO: 2 introduced into mammalian cells by transient transfection.

The effectors of SEQ ID NO: 2 was cloned into a pcda3.1 backbone (Invitrogen). The plasmid was then maxi-prepped and diluted to 1 μg/μL. For RNA guide preparation, a dsDNA fragment encoding an RNA guide was derived by ultramers containing the target sequence scaffold, and the U6 promoter. Ultramers were resuspended in 10 mM Tris·HCl at a pH of 7.5 to a final stock concentration of 100 μM. Working stocks were subsequently diluted to 10 μM, again using 10 mM Tris·HCl to serve as the template for the PCR reaction. The amplification of the RNA guide was done in 50 μL reactions with the following components: 0.02 μl of aforementioned template, 2.5 μl forward primer, 2.5 μl reverse primer, 25 μL NEB HiFi Polymerase, and 20 μl water. Cycling conditions were: 1×(30 s at 98° C.), 30×(10 s at 98° C., 15 s at 67° C.), 1×(2 min at 72° C.). PCR products were cleaned up with a 1.8× SPRI treatment and normalized to 25 ng/μL. The sequence of the AAVS1 target locus tested was GCGAGTGAAGACGGCATGG (SEQ ID NO: 218), and the corresponding crRNA sequence was

(SEQ ID NO: 219)
AUAACGACCCUGCGAAGUGGGGUGUAACUUCGACGCGAGUGAAGACGGC
AUGG.

Approximately 16 hours prior to transfection, 100 μl of 25,000 HEK293T cells in DMEM/10%FBS+Pen/Strep were plated into each well of a 96-well plate. On the day of transfection, the cells were 70-90% confluent. For each well to be transfected, a mixture of 0.5 μl of Lipofectamine 2000 and 9.5 μl of Opti-MEM was prepared and then incubated at room temperature for 5-20 minutes (Solution 1). After incubation, the lipofectamine:OptiMEM mixture was added to a separate mixture containing 182 ng of effector plasmid and 14 ng of crRNA and water up to 10 μL (Solution 2). In the case of negative controls, the crRNA was not included in Solution 2. The solution 1 and solution 2 mixtures were mixed by pipetting up and down and then incubated at room temperature for 25 minutes. Following incubation, 20 μL of the Solution 1 and Solution 2 mixture were added dropwise to each well of a 96 well plate containing the cells. 72 hours post transfection, cells are trypsinized by adding 10 μL of TrypLE to the center of each well and incubated for approximately 5 minutes. 100 μL of D10 media was then added to each well and mixed to resuspend cells. The cells were then spun down at 500 g for 10 minutes, and the supernatant was discarded. QuickExtract buffer was added to ⅕ the amount of the original cell suspension volume. Cells were incubated at 65° C. for 15 minutes, 68° C. for 15 minutes, and 98° C. for 10 minutes.

Samples for Next Generation Sequencing were prepared by two rounds of PCR. The first round (PCR1) was used to amplify specific genomic regions depending on the target. PCR1 products were purified by column purification. Round 2 PCR (PCR2) was done to add Illumina adapters and indexes. Reactions were then pooled and purified by column purification. Sequencing runs were done with a 150 cycle NextSeq v2.5 mid or high output kit.

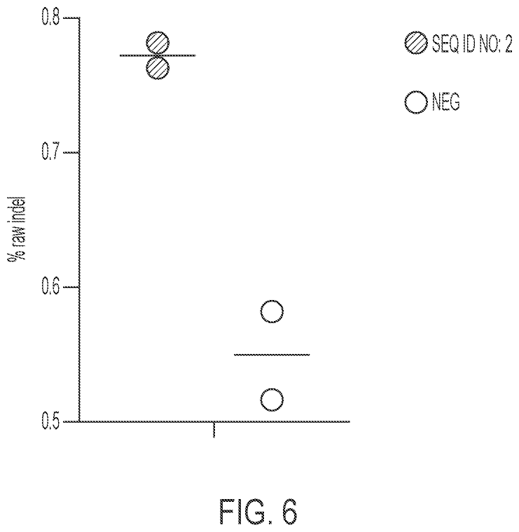

FIG. 6 shows percent indels in the AAVS1 target locus in HEK293T cells following transfection with the effector of SEQ ID NO: 2. The dots reflect percent indels measured in two bioreplicates, and the bars reflect the mean percent indels measured in the two bioreplicates. The closed dots represent indels induced by the effector of SEQ ID NO: 2, and the open dots represent indels measured in the negative control samples. For the effector of SEQ ID NO: 2, the percent indels were higher than the percent indels of the negative control.

This Example suggests that the effector of SEQ ID NO: 2 has nuclease activity in mammalian cells.

What is claimed is:

1. A composition comprising:
(a) a nuclease or a nucleic acid encoding the nuclease, wherein the nuclease comprises an amino acid sequence with at least 80% identity to any one of SEQ ID NOs: 1-37 and SEQ ID NOs: 221-224; and
(b) an RNA guide or a nucleic acid encoding the RNA guide, wherein the RNA guide comprises a direct repeat sequence and a spacer sequence,
wherein the nuclease binds to the RNA guide, and wherein the spacer sequence binds to a target nucleic acid.

2. The composition of claim 1, wherein the nuclease comprises a RuvC domain or a split RuvC domain

3. The composition of claim 1 or 2, wherein the nuclease comprises a catalytic residue (e.g., aspartic acid or glutamic acid).

4. The composition of any one of claims 1-3, wherein the nuclease comprises one or more of the following sequences:
(a) X₁X₂X₃X₄GX₅X₆ (SEQ ID NO: 233), wherein X₁ is V or A or C, X₂ is Y or F, X₃ is K or Q, X₄ is Y or F, X₅ is L or A or M or C or T, and X₆ is I or V or L;
(b) LX₁NX₂LV (SEQ ID NO: 234), wherein X₁ is W or K or R and X₂ is N or T or K or S or D or Q;
(c) FDX₁X₂G (SEQ ID NO: 235), wherein X₁ is G or Y and X₂ is T or S or M;
(d) X₁X₂HR X₃X₄P (SEQ ID NO: 236), wherein X₁ is I or L or V, X₂ is Y or L or M or F, X₃ is P or H or D or E, and X₄ is L or I or V or M;
(e) GX₁DX₂GX₃R (SEQ ID NO: 237), wherein X₁ is I or L or V, X₂ is I or V or L, and X₃ is F or Y;
(f) RX₁X₂X₃YR (SEQ ID NO: 238), wherein X₁ is K or Q or E, X₂ is H or D or E, and X₃ is F or V or L or I; and
(g) X₁DX₂DX₃NAAX₄N (SEQ ID NO: 239), wherein X₁ is H or Y, X₂ is R or Q or V, X₃ is E or T or I or H or K or Q or D, and X₄ is N or R or I or V or K.

5. The composition of any one of claims 1-4, wherein the nuclease comprises an amino acid sequence with at least 95% identity to any one of SEQ ID NOs: 1-37 and SEQ ID NOs: 221-224.

6. The composition of any one of claims 1-5, wherein the nuclease comprises an amino acid sequence set forth in any one of SEQ ID NOs: 1-37 and SEQ ID NOs: 221-224.

7. The composition of any one of claims 1-6, wherein the composition does not include a tracrRNA.

8. The composition of any one of claims 1-7, wherein the direct repeat sequence comprises one or more of the following sequences:
(a) X₁X₂CCCTX₃ (SEQ ID NO: 240), wherein X₁ is G or A, X₂ is A or C, and X₃ is G or A; and
(b) X₁GGGX₂X₃X₄X₅X₆A (SEQ ID NO: 241), wherein X₁ is T or G, X₂ is T or G, X₃ is T or G, X₄ is A or G, X₅ is T or A, and X₆ is A or G or C.

9. The composition of any one of claims 1-8, wherein the direct repeat sequence comprises a nucleotide sequence with at least 95% sequence identity to any one of SEQ ID NOs: 38-126.

10. The composition of any one of claims 1-9, wherein the direct repeat sequence comprises the nucleotide sequence set forth in any one of SEQ ID NOs: 38-126.

11. The composition of any one of claims 1-10, wherein the spacer sequence comprises between 15 and 24 nucleotides in length.

12. The composition of any one of claims 1-11, wherein the target nucleic acid comprises a sequence complementary to a nucleotide sequence in the spacer sequence.

13. The composition of any one of claims 1-12, wherein the target nucleic acid is adjacent to a protospacer adjacent motif (PAM) sequence, wherein the PAM sequence comprises a nucleotide sequence set forth as 5′-CN-3′, 5′-CCN-3′, 5′-NCN-3′, 5′-NCCN-3′, or 5′-NNCN-3′, wherein “N” is any nucleobase.

14. The composition of claim 13, wherein the PAM sequence comprises a nucleotide sequence set forth as 5′-ACCN-3′, 5′-DCCN-3′, 5′-DTTN-3′, 5′-DYYN-3′, 5′-GCCN-3′, 5′-GTTN-3′, 5′-GYYN-3′, 5′-HCN-3′, 5′-HNCN-3′, 5′-HNCR-3′, 5′-HNCV-3′, 5′-RCCN-3′, 5′-RCCR-3′, 5′-RYCN-3′, wherein “D” is A or G or T, “H” A or C or T, “N” is any nucleobase, “R” is A or G, “V” is A or C or G, and “Y” is C or T.

15. The composition of claim 13, wherein the PAM sequence comprises a nucleotide sequence set forth as 5′-CCA-3′, 5′-CCC-3′, 5′-CCT-3′, 5′-CCG-3′, 5′-ACCG-3′, 5′-CCCA-3′, 5′-CCCG-3′, 5′-TCCA-3′, or 5′-TCCT-3′.

16. The composition of any one of claims 1-15, wherein the nuclease cleaves the target nucleic acid.

17. The composition of any one of claims 1-16, wherein the target nucleic acid is single-stranded DNA or double-stranded DNA.

18. The composition of any one of claims 1-17, wherein the composition comprises at least 10% greater enzymatic activity than a reference composition, e.g., at least 10% greater nuclease activity than a nuclease activity of a reference composition.

19. The composition of any one of claims 1-18, wherein the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

20. The composition of any one of claims 1-19, wherein the nucleic acid encoding the nuclease is codon-optimized for expression in a cell.

21. The composition of any one of claims 1-20, wherein the nucleic acid encoding the nuclease is operably linked to a promoter.

22. The composition of any one of claims 1-21, wherein the nucleic acid encoding the nuclease is in a vector.

23. The composition of claim 22, wherein the vector comprises a retroviral vector, a lentiviral vector, a phage vector, an adenoviral vector, an adeno-associated vector, or a herpes simplex vector.

24. The composition of any one of claims 1-23, wherein the composition is present in a delivery vehicle comprising a nanoparticle, a liposome, an exosome, a microvesicle, or a gene-gun.

25. A cell comprising the composition of any one of claims 1-24.

26. The cell of claim 25, wherein the cell is a eukaryotic cell or a prokaryotic cell.

27. The cell of claim 25, wherein the cell is a mammalian cell or a plant cell.

28. The cell of claim 25, wherein the cell is a human cell.

29. A method of binding the composition of any one of claims 1-28 to the target nucleic acid in a cell comprising:
(a) providing the composition; and
(b) delivering the composition to the cell,
wherein the cell comprises the target nucleic acid, wherein the nuclease binds to the RNA guide, and wherein the spacer sequence binds to the target nucleic acid.

30. A composition comprising:
(a) a nuclease or a nucleic acid encoding the nuclease, wherein the nuclease comprises an amino acid sequence with at least 80% identity to SEQ ID NO: 2; and
(b) an RNA guide or a nucleic acid encoding the RNA guide, wherein the RNA guide comprises a direct repeat sequence and a spacer sequence,
wherein the nuclease binds to the RNA guide, and wherein the spacer sequence binds to a target nucleic acid.

31. The composition of claim 30, wherein the nuclease comprises a RuvC domain or a split RuvC domain

32. The composition of claim 30 or 31, wherein the nuclease comprises a catalytic residue (e.g., aspartic acid or glutamic acid).

33. The composition of any one of claims 30-32, wherein the nuclease comprises one or more of the following sequences:
(a) X₁X₂X₃X₄GX₅X₆ (SEQ ID NO: 233), wherein X₁ is V or A or C, X₂ is Y or F, X₃ is K or Q, X₄ is Y or F, X₅ is L or A or M or C or T, and X₆ is I or V or L;
(b) LX₁NX₂LV (SEQ ID NO: 234), wherein X₁ is W or K or R and X₂ is N or T or K or S or D or Q;
(c) FDX₁X₂ G (SEQ ID NO: 235), wherein X₁ is G or Y and X₂ is T or S or M;
(d) X₁X₂HRX₃X₄P (SEQ ID NO: 236), wherein X₁ is I or L or V, X₂ is Y or L or M or F, X₃ is P or H or D or E, and X₄ is L or I or V or M;
(e) GX₁DX₂GX₃R (SEQ ID NO: 237), wherein X₁ is I or L or V, X₂ is I or V or L, and X₃ is F or Y;
(f) RX₁X₂X₃YR (SEQ ID NO: 238), wherein X₁ is K or Q or E, X₂ is H or D or E, and X₃ is F or V or L or I; and
(g) X₁DX₂DX₃NAAX₄N (SEQ ID NO: 239), wherein X₁ is H or Y, X₂ is R or Q or V, X₃ is E or T or I or H or K or Q or D, and X₄ is N or R or I or V or K.

34. The composition of any one of claims 30-33, wherein the nuclease comprises an amino acid sequence with at least 95% identity to SEQ ID NO: 2.

35. The composition of any one of claims 30-34, wherein the nuclease comprises the amino acid sequence set forth in SEQ ID NO: 2.

36. The composition of any one of claims 30-35, wherein the composition does not include a tracrRNA.

37. The composition of any one of claims 30-36, wherein the direct repeat sequence comprises one or more of the following sequences:
(a) X₁X₂CCCTX₃ (SEQ ID NO: 240), wherein X₁ is G or A, X₂ is A or C, and X₃ is G or A; and
(b) X₁GGGX₂X₃X₄X₅X₆A (SEQ ID NO: 241), wherein X₁ is T or G, X₂ is T or G, X₃ is T or G, X₄ is A or G, X₅ is T or A, and X₆ is A or G or C.

38. The composition of any one of claims 30-37, wherein the direct repeat sequence comprises a nucleotide sequence with at least 95% sequence identity to SEQ ID NO: 40 or SEQ ID NO: 41.

39. The composition of any one of claims 30-38, wherein the direct repeat sequence comprises a nucleotide sequence set forth in SEQ ID NO: 40 or SEQ ID NO: 41.

40. The composition of any one of claims 30-37, wherein the direct repeat sequence comprises a nucleotide sequence with at least 95% sequence identity to SEQ ID NO: 111.

41. The composition of any one of claim 30-37 or 40, wherein the direct repeat sequence comprises the nucleotide sequence set forth in SEQ ID NO: 111.

42. The composition of any one of claims 30-41, wherein the spacer sequence comprises between 15 and 24 nucleotides in length.

43. The composition of any one of claims 30-42, wherein the spacer sequence comprises about 19 or nucleotides in length.

44. The composition of any one of claims 30-43, wherein the target nucleic acid comprises a sequence complementary to a nucleotide sequence in the spacer sequence.

45. The composition of any one of claims 30-44, wherein the target nucleic acid is adjacent to a PAM sequence, wherein the PAM sequence comprises a nucleotide sequence set forth as 5′-CN-3′, 5′-CCN-3′, 5′-NCN-3′, 5′-NCCN-3′, or 5′-NNCN-3′, wherein “N” is any nucleobase.

46. The composition of claim 45, wherein the PAM sequence comprises a nucleotide sequence set forth as 5′-ACCN-3′, 5′-DCCN-3′, 5′-DTTN-3′, 5′-DYYN-3′, 5′-GCCN-3′, 5′-GTTN-3′, 5′-GYYN-3′, 5′-HCN-3′, 5′-HNCN-3′, 5′-HNCR-3′, 5′-HNCV-3′, 5′-RCCN-3′, 5′-RCCR-3′, 5′-RYCN-3′, wherein “D” is A or G or T, “H” A or C or T, “N” is any nucleobase, “R” is A or G, “V” is A or C or G, and “Y” is C or T.

47. The composition of claim 45, wherein the PAM sequence comprises a nucleotide sequence set forth as 5′-CCA-3′, 5′-CCC-3′, 5′-CCT-3′, 5′-CCG-3′, 5′-ACCG-3′, 5′-CCCA-3′, 5′-CCCG-3′, 5′-TCCA-3′, or 5′-TCCT-3′.

48. The composition of any one of claims 30-47, wherein the nuclease cleaves the target nucleic acid.

49. The composition of any one of claims 30-48, wherein the target nucleic acid is single-stranded DNA or double-stranded DNA.

50. The composition of any one of claims 30-49, wherein the composition comprises at least 10% greater enzymatic activity than a reference composition, e.g., at least 10% greater nuclease activity than a nuclease activity of a reference composition.

51. The composition of any one of claims 30-50, wherein the nuclease further comprises a peptide tag, a fluorescent protein, a base-editing domain, a DNA methylation domain, a histone residue modification domain, a localization factor, a transcription modification factor, a light-gated control factor, a chemically inducible factor, or a chromatin visualization factor.

52. The composition of any one of claims 30-51, wherein the nucleic acid encoding the nuclease is codon-optimized for expression in a cell.

53. The composition of any one of claims 30-52, wherein the nucleic acid encoding the nuclease is operably linked to a promoter.

54. The composition of any one of claims 30-53, wherein the nucleic acid encoding the nuclease is in a vector. The composition of claim 54, wherein the vector comprises a retroviral vector, a lentiviral vector, a phage vector, an adenoviral vector, an adeno-associated vector, or a herpes simplex vector.

56. The composition of any one of claims 30-55, wherein the composition is present in a delivery vehicle comprising a nanoparticle, a liposome, an exosome, a microvesicle, or a gene-gun.

57. A cell comprising the composition of any one of claims 30-56.

58. The cell of claim 57, wherein the cell is a eukaryotic cell or a prokaryotic cell.

59. The cell of claim 57, wherein the cell is a mammalian cell or a plant cell. The cell of claim 57, wherein the cell is a human cell.

61. A method of binding the composition of any one of claims 30-60 to the target nucleic acid in a cell comprising:
(a) providing the composition; and
(b) delivering the composition to the cell,
wherein the cell comprises the target nucleic acid, wherein the nuclease binds to the RNA guide, and wherein the spacer sequence binds to the target nucleic acid.


SEQUENCE LISTING (250 sequences)

SEQ ID NO: 1 - protein, 575 aa, Artificial Sequence
  Description of Artificial Sequence Synthetic polypeptide
MIKAYKFGLLNPISGFDQAAMDVLYLRNKLWNQLVELEKNSRAAYRALMLDSSEELSVIQTRIDAIEVERADLVSQKKKLRASVRSKKVDTAGIDAAVERLIAERTNLRAKAKQLREVVKVEIKPKAVELDKVRYAAVLALIKGSGLWWGNSETVIAAYDVARVRAMKESAELRFRSFDGTGKFAYRESGGIDFDKFMSGKVNFARLNTLPDSDFAHLSERGRRSKARHHLTMTVLTSVDDAGKKVRHEVTWPIVMHRDMPAGAIKTIHVHRKRVGDQFNWTCSITIDVPEEPKQLIDHPAKAACGIDLGFRLVKDGLRIATIADSDNRIEHVVLPLDWIEKMDYVEHLQSTLSETANLTWVRLRKHLSELPDYPESIKERIHNILKAGERVPTRGMRSLLGALKAEPELLPEALQILAAWSDDIYRPAREMHNLRDKLMKRRQDLYRNVSHCLSNKYAMVRVEDMDLRQIARVKKDDGSDNPLPDTVRDNRKRAALFEFVLSIKQSCVKTGSVFEKMNPAYSSMTCSSCGHLNQPGMDIHYSCENCGTLHDQDENAAKNFLRGEYFSSPKQDVA

SEQ ID NO: 2 - protein, 604 aa, Artificial Sequence
  Description of Artificial Sequence Synthetic polypeptide
MAVEERTKKGVSVRVYKYGLVPKGCLPDEAKDELLRANNLWNKLVEISRKNQSDFDEVRKKAHPPYGEEMVRLETINEKIDDAYDRKRDARKEAGTRDETHPLIIKANAVIEILKEERSEIYDTLKLLRTEADKSVDKKALNESFKSNIKLARRETSLNSDTKEEIIRNDFRTARDRTFKTGGRLRFHTFDGTGYWHFRFRRNDEKGKKVDDYTIDDLFAGEKPSPKKPLPNRFKFLSRDDTRRKPRLRLRTTLAGGRTNASKVFQEFDVIYHRPVPEGAIIPNAKILRTRTGDKFRYDLVLTVYLPEPKHKDIPLDDAIGIDLGFREDPDDKYRKQVGAIISLDPSDEVEEIFAPPKMVKAFGHIDELKSVLDESAADLGMKIKPLMKDIRLPEDNEKYKQEYKLWNSIVNARANVTLSFEKAYKLALWCKKKDAGIPEDITGPVVYWWKGYSRRYRELHNLRKKQLLNRKDFYRQIASRLVLRGLLIGVENFNLSKIARSKDEDNVLNNKARANRFLLSPSEFRAAIKNAADREGIPCLEVNPANTSKICFDCGTLNKNLGSEKNWVCPACGCVHDRDTNAARNIAKRALEKYLEDRKSAGK

SEQ ID NO: 3 - protein, 586 aa, Artificial Sequence
  Description of Artificial Sequence Synthetic polypeptide
MRTRVYKYGLVPIGYPPQEAVDELWRANNLWNTLVALHRESRENWDDARRAASIAYSEKMDALEKKQAEISQAFDALQQVRMEEGTKDESNPKLKLQRDVINRLKKEQGVIFDELKPLRKDADKVLDTKQLNDDFRKKVNTAASVKNNGLYNPTADEVVRNFKEARQKVLSNPAKSKLNIHSFDGTGYFNFRFRRKGEKQDGVWFSELFAGNSKEDRRFAILSKDESRKKIRLRLRVIMAGGATKASKVYQEFDWIYHRPIPEDAQIQNGKILRTRAGDKCRYDLVLTLRLPDIDKIAPNDLDGTIGIDIGFRKSGDTVLIATVMSDEVSSKPQEIKAPTEMVSALEHVINLQSELDDAATDLGRTITPLLKANPPPQDHQKYRLWKSIAQRPSNETLSYEKAYKFAIWLNNEPDTFPKEITEKVHTWWRSYSRKYREIHNRRRKQLTHRKHFYREAAASIVAQRKLIVLEKIPLNKFAETKDKDTKLNNRARAQRFLASLSEFREAIKNAADREGVPCIEVNPAYTSKTCSDCGSLNKALKAEKKWTCPSCGVVHDRDTNAANNLQKMGQIYIDDVKKTINEVLE

SEQ ID NO: 4 - protein, 601 aa, Artificial Sequence
  Description of Artificial Sequence Synthetic polypeptide
MAEAKAFTTRVYKYGAIPLGLFPKEGVDQLFRANQLWNKLVEINNEHAEYYDQARRDVDEEYNTLSVELDNVEDRVDKAFTGKRNARMKASSRDADHPLVKTANDKIDTLFAERRELWDGIKQPRSRADKVIDKDVLNTAFNTAVKEAKRSDNTDGLDSNTANEVWRYFKEARGKVFQNPRSKLRFHRFDGMGYRFYRFRDRLIKTNKDGVSFEYFSRRGGDDDRAFLLTPSAKEPSTRDKRRGVQRYHLKLKVAGGHSKASKVYAEFDLLMHRHIPDGAQINNAKLMRIRTGDRFKYTVNFSVRVPVAEPAKPKAHAIGVDIGFRRLEDGSIRAATIAGTSSDFETLSVGLDQEYLDRIEHIESLQTKMDERATELGKKIKPLLKAGAVLKEGHDFYKFVKAIAVPRRKDVTMSLEQAYKLGSWLKNNPTELPKAVVEPVFDWWDHNAKHYREMHNLRRKTLAWRKEEYRFLAHGLVGHGLPIGIEAIDLSTFAEVKDKDNKLRNKALSQRFLVSNSELIGAIKNAAQREGVPVIEVPAPYTSKTCSACGNVHKELKAELEWDCPECGVVHDRDENAAVNIARSAVKKMAPKKPKALAAE

SEQ ID NO: 5 - protein, 604 aa, Artificial Sequence
  Description of Artificial Sequence Synthetic polypeptide
MAVEERTKKGVSVRVYKYGLVPKGCLPDEAKDELLRANNLWNKLVEISRKNQSDFDEVRKKAHPPYGEEMVRLETINKKIDDAYDRKRDARKEAGTRDETHPLIIKANAVIEILKEERSEIYDTLKLLRTEADKSVDKKALNESFKSNIKLARRETSLNSDTKEEIIRNDFRTARDRTFKTGGRLRFHTFDGTGYWHFRFRRNDEKGKKVDDYTIDDLFAGEKPSPKKPLPNRFKFLSRDDTRRKPRLRLRTTLAGGRTNASKVFQEFDVIYHRPVPEGAIIPNAKILRTRTGDKFRYDLVLTVYLPEPKHKDIPLDDAIGIDLGFREDPDDKYRKQVGAIISLDPSDEVEEIFAPPKMVKAFGHIDELKSVLDESAADLGMKIKPLMKDIRLPEDNEKYKQEYKLWNSIVNARANVTLSFEKAYKLALWCKKKDAGIPEDITGPVVYWWKGYSRRYRELHNLRKKQLLNRKDFYRQIASRLVLRGLLIGVENFNLSKIARSKDEDNVLNNKARANRFLLSPSEFRAAIKNAADREGIPCLEVNPANTSKICFDCGTLNKNLGSEKNWVCPACGCVHDRDTNAARNIAKRALEKYLEDRKSAGK

SEQ ID NO: 6 - protein, 587 aa, Artificial Sequence
  Description of Artificial Sequence Synthetic polypeptide
MTTRVYKYGLIPIGYPPQAAIDELFRANNLWNTLVALHRESRENWDDARRSASILYSEKMDELDKKNEDISEAFNGLNQARMDEGTKDETGNKRLQAERAIINRLKKEQKEIYAELKPLRKEADKTVDRKALNDEYRNKCNTAVSAKVSGVYSRTAGELYAYFRTARDKAFKENATLRFHRFDGTGYFAFRCRSKTVGVNVDGISVEDFMSKQFIDYLRCAVQSVDESRKKPRIRINAVLTGGRTIASKVTQEFDWIYHRPLPPEGQIQNGKILRTRVGDKFKYDLVLTVKLPDVEMIKPAALNGTIGIDVGFRKVGNSLLIGTVMSSDSAQKAVALEVPAMMVSALEHVVALQSELDDAASDLGKAITPLLKANPIDEEHGKYRLWRSLALRPLHVTLSFEQAYKLALWLKREPNLFPSEINEKVHTWWRSYSRKYRESHNRRKKQLTHRKHFYRETAAKLVAQNRLIVLEKINLTDFAETKNKNTKLSNKARAQRFMASLGEFRDAIKNAADREGVPVIDVNPAYTSKTCSECGYLNKELKSEKEWNCPECGVVHDRDENAANNLQKMGQKYLLDAAKTAPVVVK

SEQ ID NO: 7 - protein, 589 aa, Artificial Sequence
  Description of Artificial Sequence Synthetic polypeptide
MVSRQTDKDKTEVRVYKYGLIPVGYPPEETISELWRANNLWNTLVGLHYKHREKYEEARCEAHLLYGEVAERLAAIDEKIEQAYDDKRTARMKAGTRDAADPLIKEANAVIKALKDKRKTIYEELKPIRIEADKRVDRSVLNSAFTKDINEATQSKNNGLYNTTANEVKRYFETARDRSFKTNSKLRFHRFDGTGFYAFRFRRKDNKVDGVSFEELFQGNKETDQRFTFIGRDESRKKTRLRLRATLAGGARKASKIKHEFDLIYHRPIPDGSKIQNGKIMRTRIGDRFKYHLVLTIKQPKSEPLIVPKDVALGIDIGFRRIKDSIQVASITSSDPSLPSQNVMAPNKMIAGMERVIELQSILDDAASELGKLTKPILNDNPQLEDHKRFGLWKAMARYPNNVTLSFETAYKVARWLQSEADFIPPKAAKLVLEWWSAYSRRYRELHNLRAKQLLHRKHFYRQVASDLVAHRQLIVLEEINLSVFAEVKDRDNNLSDKARAQRFLAAPSEFRDAICNAAQREKVPFVTVPPQYTSKMCSSCSVINRELDSQKKWECPSCGVTHDRDENAARNIAKLGQKYFSGEKKKKK

SEQ ID NO: 8 - protein, 582 aa, Artificial Sequence
  Description of Artificial Sequence Synthetic polypeptide
MINCYKFGCLQPTAGFDQSAIEHLFLRNKLWNTLVALDHEFRQRYRDLMLNSDEKLKSVQDSIDSINQEIEDLVENKMKLRQKERTKNIDSKLLDERINVLKAKRKTLSADSKTERERVKVEIKPQIDLLNTERYEAKKLAYKESGLWWGNYETVVAAYDTASQKAMKSNTELRFKSFDGSGKFAVRFEDGGLTIDELKAGASNLCRIETLNTSAFQNLSQRSIKSKARHSLTMTIYTFNDEKGKKQRKEITVPIIFHREMEEGKIKTIHLQRKRLGNQFTWSASFTLKNDIEPANVADHPATASCGIDLGYRLVKDGLRVATVADSQNNVEYLVLPKSWIDRMDYTETLQSGLSEAMTLMWAKLKAEIAKIPEYPDAVAEIIKNMQKMGDRLPYKGIKRLYRVLKEQDSTGSPVAGFNAVLDILKAWDKATYRQELEMVNLKDKLLKQREHIYRNFAAGLTKKYAHIVVEDMGLAELAKTEKSETETNDMPNAVKANRQRASLYSLVEAIRLSAAKVGSYFEKSKAAYSSMTCNVCGHLNPKTQNIHQSCESCNTMYDVDENAARNFLKGEYINEKVLKQG

SEQ ID NO: 9 - protein, 590 aa, Artificial Sequence
  Description of Artificial Sequence Synthetic polypeptide
MKRVTETDFTTRVYQYGVVPIDTFPEEGVEELFRANKLWNDLVAFNRDHREAYDQARSDADEEYASLLQSLEKLNEKVDKAYDNKRNARLKAGTKDATHPLIKEANAKIRDLQIERRELWEPLKTARKSADKKLDKKSFNDSFRITTNELQQVKNTDGLQNSTANLVADYFRTAKDRTFKDPKAKLQFHRFDGTGFFFYRFRKTGSNVDGVNFDELFARDEKDARPFVFLSTDKSRSKPRLRLRIKVAGGQSVASKTYMHFDLILHRPVPMNAQVQSGKVVRKRVGDKFSHTVSLTLRIPENGTVRLKKKAIGIDIGFRQSGKIIRAAAIASSDPKDPVEYIDVSEKFLKRIEYIDSLKSRLDEKATRLGEIIKPLLKKGKVLPEDHKKYRFVKSIANTPANVTLSFEKAYKLGSWMVKYGKGELPIEVEQEGIKWWEKNSLTYREHHNLRKKAYLERKDQYRNIASNLIKKKQPIGIEQINLSVFAETKDKDNKLGNVARLNRFLVAPSELLNAIKNAAQREGVPVFEVPAKNTSKTCSSCGHIHKELGAEPNWTCPSCKAEHDRDHNAAINIARRAEEELKKIDEKTK

SEQ ID NO: 10 - protein, 585 aa, Artificial Sequence
  Description of Artificial Sequence Synthetic polypeptide
MATRVYKYGLIPIGYPPKETIDELFKANVLWNNLVALHRKNREDWDDARRAASILYSDKIDELEKKEEDLDAAWKAFQQARMDEGTRDETNNKRLKSERASINRLKAERAEIYKELKPLRKEADKEIDKKQLNDSFRAQVNEALSVNNSGVYRAIADQIYENFKTAKDKSIKENATLRFHRFDGTGYYHFRCRRKGTNVDGISIDDFMSRNFEAYPRCAVQNIDNSKKKPRIRINAVLAGGKSKASKIHQEFDLIYHRPLPIDAQIQNGKILRTRVGDKFKYDLVLTLKIPDKEPISYNNLKGTIGIDIGFRRSVNSLLIGTVMSSDVTEEAYEIIVPPKIVEAFEHVIDLQSELDDAATDLGRIITPLLKAHPLDEDHSKYKMWRSLALRPAHVTLSFEQAYKLAIWLKHEPDTFPEEITKKVHTWWRSYSRKYRELHNRRRNQLTHRKHFYREEAAKIVALNKLIVLEEINLTDFAETKEKNTKLSKKARAQRFMASLSEFRDAIKNAAQRDGIGIIDVNPAYTSKTCSECGNLNKDLRSEKQWSCPACGVVHDRDENAANNLQKMGQSYLENIKKETSEIIE

SEQ ID NO: 11 - protein, 585 aa, Artificial Sequence
  Description of Artificial Sequence Synthetic polypeptide
MATRAYKYGLIPIGYPPKETIDELFKANVLWNNLVALHRKNREDWDDARRAASVLYSDKIDELEKKEEDLDAAWKAFQQARMDEGTRDETNNKRLKSERASINRLKSERAEIYKELKPLRKEADKEIDKKQLNDSYRAQVNEALSVRNSGIYNATAGQIYENFKTAKDKSIKENATLRFHRFDGTGYYHFRCRRKGAKVDGISVDDFMSRNFIANPRCAVQSIDNSKKKPRIRIDAVLVGGITKASKIHQEFDLIYHRPLPIDAQIQNGKILRTRVGDKFKYDLVLTLKIPDKELISYNNLKGSIGIDIGFRRSVNSLLIGTVMSSDVTEKAYEIIVPPKIVEAFEHVIDLQSELDDAATDLGRIITPLLKAHPLDEDHSKYKMWRSLALKPAHVTLSFEQAYKLAIWLKHEPDTFPEEITKKVHTWWRSYSRKYRELHNRRRNQLTHRKHFYREEAAKIVARNKLIVLEEINLTDFAETKEKNTKLSKKARAQRFMASLSEFRDAIKNAAQRDGIGFIDVNPAYTSKTCSECGNLNKGLRSEKQWSCPACGVVHDRDENAANNLQKMGQTYLESVKKETSEVIE

SEQ ID NO: 12 - protein, 570 aa, Artificial Sequence
  Description of Artificial Sequence Synthetic polypeptide
MKRQAENVRSMVYQYGTVPARVAPVEGEELALSQMRLAQRLWNVLVTIERARVAGYRSIMRDEVQEQIDALRERKDATWQEIKATRQKARAKVATPGLDAEMMRIKTALRLLVEHKQSTKQQRHDARREQLNALAERANQRIKRARQAAASMGLFWGTYNAVIQSADAGRKHAGELRYQGFRGEGTVTAQVMGGATPEQCVAGGHPFFQVAPATPGQKWRYARVRIGSTSERQPLWVAIPVVYHREIPAEARIKSVSATRRILAGKVRWSLNVTVTLPPAEPRPAGQMVAIDIGWRLLPDGVRVAYWQDGTGNHSEVRIADSDIAQFRKISDLRSICDRAREEFLPSLVEWLKPYELDEEWTHRARALAQWRSNDRIAALIRWWADHRLSGDAEIYQTAVEWRRQYLHLANWWRNQQEQMTLRVREQYRRFAAGIASQFATVIVEDFDLRQVTETTEKAVGTYRQMVSPSLFRAAVINACKREGVEIRIVSGAYSTGACHNCQHIEVWDQAASILHRCGACGALWDQDHNAAINLLASGGVVLWRINLVAAIGPLSQDRSQTGGKEAVES

SEQ ID NO: 13 - protein, 598 aa, Artificial Sequence
  Description of Artificial Sequence Synthetic polypeptide
MVSRQRKDSNKRTLVYQYGAVPIGSFPEEGLNELWRTNQLWNQLVELHNKNRQSYESARRAASDAYALVSESIALKQAEIDQAFKDKRSARMQAGTKDADHPLIATANAVIDTLMAERKTLYDQAKPLRKQADEGLDKKALADQFQEAVKTARRVQQSGISSVLADQVVAYFQTAREKAFKDAATLRFHRFDGSGYFFYRFRRPGSTSDGVSFKELFSDDPNNSEAFVFLSRDDSRPKKPRLRLRVKVAGGQTKASKRYANFDLILHRPLPKDAQIQNAKLNRVRTGDKFDYRVSFTVKEEMPQAPQLQAGAIGVDIGFRQTTSGLLRAAALAFVDAKAQPLGEVRFIDVDPALLSRVGDQGHVNALKAQLDDAAAALGRDIVPLLKAGAVLPEDHPKYKMVKAAANLPPNVTLSYERAYKLSTWSHFEPDALPLAVVNALAHWRTTYKHRYRELHNLRAKALLQRKHSYRQIAAELVRYRLPIGIELIDLSKFAETKDADNKLGNTARANRFTVSPSELLNAIKNAGEREGVPVYEVPPRDTSKRCHHCGHVHRALRAEQMWTCPNCATQHDRDHNAAINIARLALEKNSTLTSESA

SEQ ID NO: 14 - protein, 604 aa, Artificial Sequence
  Description of Artificial Sequence Synthetic polypeptide
MAVEERTKKGVSVRVYKYGLVPKGCLPDEAKDELLRANNLWNKLVEISRKNQSDFDEVRKKAHPPYGEEMVRLETINEKIDDAYDRKRDARKEAGTRDETHPLIIKANAVIEILKEERSEIYDTLKLLRTEADKSVDKKALNESFKSNIKLARRETSLNSDTKEEIIRNDFRTARDRTFKTGGRLRFHTFDGTGYWHFRFRRNDEKGKKVDDYTIDDLFAGEKPSPKKPLPNRFKFLSRDDTRRKPRLRLRTTLAGGRTNASKVFQEFDVIYHRPVPEGAIIPNAKILRTRTGDKFRYDLVLTVYLPEPKHKDIPLDDAIGIDLGFREDPDDKYRKQVGAIISLDPSDEVEEIFAPPKMVKAFGHIDELKSVLDVSAADLGMKIKPLMKDIRLPEDNEKYKQEYKLWNSIVNARANVTLSFEKAYKLALWCKKKDAGIPEDITGPVVYWWKGYSRRYRELHNLRKKQLLNRKDFYRQIASRLVLRGLLIGVENFNLSKIARSKDEDNVLNNKARANRFLLSPSEFRAAIKNAADREGIPCLEVNPANTSKICFDCGTLNKNLGSEKNWVCPACGCVHDRDTNAARNIAKRALEKYLEDRKSAGK

SEQ ID NO: 15 - protein, 587 aa, Artificial Sequence
  Description of Artificial Sequence Synthetic polypeptide
MTTRVYKYGLIPIGYPPQVAIDELFRANNLWNTLVALHRESRENWDDARRSASILYSEKMDELDKKNKDIREAFNGLNQARMDEGTKDETGNKRLQAERAIINRLTKEQKEIYAELNPLRKEADKTVDKKALNDEYRKKCNTAVSAKVSGVYSRTAGELYAYFRTARDKAFKDKTTLRFHRFDGTGYFAFRCRSKAVGVNVDGISVEDFMSQGFMDYMRCAVMSIDESKKKPRIRISAVLTGGATKASKVVQEFDWIYHRPLPPEGQIQNGKILRTRVGDKFKYDLVLTVKLPDVEMIQPAALNGTIGIDVGFRKVGNSLLIGTVMFSDSAQKAVALEVPTMVVSALEHVDALRSELDDVASDLGKAITPLLKANPIDEEHDKYRLWRSLALRPLHVTLSFEQAYKLALWLKREPNLFPSEINEKVHTWWRSYSRKYREIHNRRKKQLTHRKHFYRETAAKLIAQNKLIVLEKIDLTDFAETKNKNTKLSNKARSQRFMAALGEFRDAIKNAADREGVPVIDVNAAYTSKTCSECGYLNKELKSEKEWNCPECGVVHDRDENAANNLQKMGQKYLLDAAKTAVVVVK

SEQ ID NO: 16 - protein, 592 aa, Artificial Sequence
  Description of Artificial Sequence Synthetic polypeptide
MAPARQRGTEALSTFVYQYGAIPKGPFPEQGIESLYKENKLWNSLVEIHNRHRESYEAARCAADSEYALISESIKRAEAAVEAAFEAKREARKQAGTRDASHPLIRAAIDEINRLKQNRRELWATAKAARKRADGSIDKAALNKAFRDAVNAAQRVGNTGGLNSTTANQVADNFRTARERAFKEGARLRFHAFDGTGYWFFRFRVKGSKVDGISYGELFSQRVDDGRSFVLTSAESDRKKPRIPLRVKVAGGAKEGSKVYAFFDLILHRPLPENAQVQNAKLIRKRNGDKFSYLVSLTVRVPRSMATEVQAAAIGVDIGFRQLADQRVRVAAIGGSEPEDECQIVEVSKEFIRRLEHVDALKASLDQSAEALGQFIKPLLKAGSVLPEDHPRYRFVRSIAAAPPIVTMSFEKAYKLARWLLREPGALPPDVEKRALDWWQQNGRRYRELHNLRQKALAARKEEYRKIAAKIVKFGRPIGVEMIDLRVFAEAKDRDNKLGNTARSNRFLVAPSELLAAIRNAATKAGIPFHEVSARNTSKTCSACGVVNEKLGAEDSWTCAVCGVVHDRDKNAAVNIARRAKEKRAPAAEGDS

SEQ ID NO: 17 - protein, 587 aa, Artificial Sequence
  Description of Artificial Sequence Synthetic polypeptide
MTTRVYKYGLIPIGYPPQVAIDELFRANNLWNTLVALHRESRENWDDARRSASILYSEKMDELDKKNKDIREAFNGLNQARMDEGTKDETGNKRLQAERAIINRLTKEQKEIYAELNPLRKEADKTVDKKALNDEYRKKCNTAVSAKVSGVYSRTAGELYAYFRTARDKAFKDKTTLRFHRFDGTGYFAFRCRSKAVGVNVDGISVEDFMSQGFMDYMRCAVMSIDESKKKPRILISAVLTGGATKASKVVQEFDWIYHRPLPPEGQIQNGKILRTRVGDKFKYDLVLTVKLPDVEMIQPAALNGTIGIDVGFRKVGNSLLIGTVMFSDSAQKAVALEVPTMVVSALEHVDALRSELDDVASDLGKAITPLLKANPIDEEHDKYRLWRSLALRPLHVTLSFEQAYKLALWLKREPNLFPSEINEKVHTWWRSYSRKYREIHNRRKKQLTHRKHFYRETAAKLIAQNKLIVLEKIDLTDFAETKNKNTKLSNKARSQRFMAALGEFRDAIKNAADREGVPVIDVNAAYTSKTCSECGYLNKELKSEKEWNCPECGVVHDRDENAANNLQKMGQKYLLDAAKTAVVVVK

SEQ ID NO: 18 - protein, 600 aa, Artificial Sequence
  Description of Artificial Sequence Synthetic polypeptide
MISRVYKYGAVPLKKFPEVKFPREQFPEEGVEELRRANKLRNSLVWLHRKNNEKFEAARVAADAEYGEIAEKLDALEKTISQALTAKRQARAKAGTRDAKHPLVKAASETINELTKQRSDLWKALKPARIRADKRVDRKALTKQFDDAVKVVQHVKETGGLSSHCANEIVRYFKESRSRALNERATLRYRRFDGTGFWFYRFREPGVNKNGVDFDGLLTGNKTEARDNRNFVLTEKSRRGKRVIYKLRAKIAGGAKKDSKVYGHFDLILHRPIPENARIQSAKILRHRTGDKFTYTVSFTLKLPDVEQQTVEGSVLGLDIGFREMERNNSYRIATLATNDQSRRVETIDIARENRRGFLARMNHIDDLRSTMDENATELGKKLLPLLKTAKPLPDSHQQFIFTERLRKTRANVTLDFERSYKMARWFIRAPDEADFYGPEIVGMVLRWWEENSFKYREMHNLRRKALAERKEVYRMEAARLVGFGIPIAVEKLDMSKWAERKDSDNELSNRALSSRFLVAPSELIAAIENAAKREGVPFIKVNAANTSKACHACGTINKALKGELIWTCEECETKHDRDINAAINIAKRGILQAKKEKKQ

SEQ ID NO: 19 - protein, 577 aa, Artificial Sequence
  Description of Artificial Sequence Synthetic polypeptide
MIKAFKFGMLAPVSGFDQAALDVLYLRNKLWNALVEQERKHRERYRALLTGSNDELSSIQSRLDEIERERSDLVARKKKVRAKVRSKNVDTSEIDAAIDVLIAERDELRGKAKALRAEVKEKIKPLIANLDRERYETIKQLTKESGLWWCNYETVVAAYDVARVKAMKANAELRFRSFDGTGKFAVRQSGGFPLSDLTAGKLSFARLEALPEDGFAHLSERGKRSMARHHLTMTIMTYKDESGKLRRHEVTWPIILHRPLPYGTVKFIYVQRKRVGKDFKWTCSITMEIDEIEKTLIDHTSRAACGIDLGYRLVTDGLRVATLADTEGKVRHLVLPQDWLDRMDHVERIQGWLSDQNNLAWEKLKALLIALQEYPEPLAESIGRLLKAGDKTPVRGMRALHWRLRNEPEIMPEALKVLDEWEVKTRRREQEMYNLRDKLINRRKDIYRNFAYQVANGYALIRIEDIQLKKLAMVKFDDGRDNPLPQAVRNNRTRAALYELALYIQQAAAKTGAEFEKMPAMNSTLICSNCGHQNTGMDKRDIHFRCEKCDTLHDQDQNAAKNFLRGQEFYLAEQMVA

SEQ ID NO: 20 - protein, 585 aa, Artificial Sequence
  Description of Artificial Sequence Synthetic polypeptide
MATRVYKYGLIPIGYPPKETIDELFKANVLWNNLVALHRKNREDWDDARRAASILYSDKIDELEKKEEDLDAAWKAFQQARMDEGTRDETNNKRLKSERASINRLKAERAEIYKELKPLRKEADKEIDKKQLNDSYRAQVNEAISVRNSGIYNATAGQVLDNFKAARDRSFKENATLKFHRFDGTGYYHFRCRRRGAKVDGINVEDFMSRNFIANPRCAVQSIDNSKKKPRIRINAVLAGGQSKASKVHQEFDLIYHRPLPIDAQIQNGKILRTRVGDKFKYDLVLTLKIPDKEPISYNNLKGTIGIDIGFRRSVNSLLIGTVMSSNVSEKAYEIKVPPKIVEAFEHVIDLKSELDDAATDLGRIITPLMKAHPLDEDHSKYKMWRSLALRPAHVTLSFEQAYKLAIWLKHEPDTFPEEITKKVHTWWRSYSRKYRELHNRRRNQLTHRKHFYREEAAKIVALNKLIVLEEINLTDFAETKEKNTKLSKKARAQRFMASLSEFRDAIKNAAQRDGIGIIDVNPAYTSKTCSECGNLNKDLRSEKQWSCPACGVVHDRDENAANNLQKMGQTYLESLKKETSEVIE

SEQ ID NO: 21 - protein, 604 aa, Artificial Sequence
  Description of Artificial Sequence Synthetic polypeptide
MAVEERTKKGVSVRVYKYGLVPKGCLPDEAKDELLRANNLWNKLVEISRKNQSDFDEVRKKAHPPYGEEMVRLETINEKIDDAYDRKRDARKEAGTRDETHPLIIKANAVIEILKEERSEIYDTLKLLRTEADKSVDKKALNESFKSNIKLARRETSLNSDTKEEIIRNDFRTARDRTFKTGGRLRFHTFDGTGYWHFRFRRNDEKGKKVDDYTIDDLFAGEKPSPKKPLPNRFKFLSRDDTRRKPRLRLRTTLAGGRTNASKVFQEFDVIYHRPVPEGAIIPNAKILRTRTGDKFRYDLVLTVYLPEPKHKDIPLDDAIGIDLGFREDPDDKYRKQVGAIISLDPSDEVEEIFAPPKMVKAFGHIDELKSVLDESAADLGMKIKPLMKDVRLPEDNEKYKQEYKLWNSIVNARANVTLSFEKAYKLALWCKKKDAGIPEDITGPVVYWWKGYSRRYRELHNLRKKQLLNRKDFYRQIASRLVLRGLLIGVENFNLSKIARSKDEDNVLNNKARANRFLLSPSEFRAAIKNAADREGIPCLEVNPANTSKICFDCGTLNKNLGSEKNWVCPACGCVHDRDTNAARNIAKRALEKYLEDRKSAGK

SEQ ID NO: 22 - protein, 585 aa, Artificial Sequence
  Description of Artificial Sequence Synthetic polypeptide
MATRVYKYGLIPIGYPPKETIQELFRANVLWNNLVALHKKNREDWDDARRAASILYSDKIDELEKNKEDLDAVWKAFNQARMDEGTRDETNNKRLKSERASIDRLDAVRAEIYKELKPLRKEADKEIDKKQLNDSYRRKVNEAVSVRNSGIYNATAGQVLDNFKTARDRSFKENATLKFHKFDYTGYYHFRCRRKGAKVDGISVDDFMSRNFIANPRCAVQSIDNSKKKPRIRINAVLVGGQSKASKIHQEFDLIYHRPLPIDAQIQNGKILRTRVGDKFKYDLVLTLKIPDKEPISYNNLKGTIGIDIGFRRSVNTLLIGTVMSSDLTEKAYEIKVPPKIVEAFEHIIDLQSELDDAATDLGRIITPLLKAHPLDEDHSKYKMWRSLALRPAHVTLSFEQAYKLSIWLKDEPDTFPEEITKKVHTWWRSYSRKYRELHNRRRNQLTHRKHFYREEAAKIVALNKLIVLEEINLTDFAETKEKNTKLSKKARAQRFMASLSEFRDAIKNAAQRDGIGIINVNPAYTSKTCSECGNLNKDLRSEKQWSCPACGVVHDRDENAANNLQKMGQTYLESLKKETSEVIE

SEQ ID NO: 23 - protein, 607 aa, Artificial Sequence
  Description of Artificial Sequence Synthetic polypeptide
MAQPHLFLLYTQAASSIIFDMTTRVYKYGLIPIGYPPKAAIDELFRANNLWNTLVALHRESRENWDDARRSACILYSEKMDELDKKNEDISEAFNGLNQARMDEGTKDETGNKRLQAERAIINRLKKERNEIYAELKPLRKEADKTVDRKALNDEYHKKCNRAVSAKVSGVYSGTAGELYAYFKTARGKAFKENATLRFHRFDGTGYFAFRCRSKTVGVNVDGISVEDFMSQGFLDYMRCAVMSVDESKKKPRIRISAVLTGGATKASKVFQEFDWIYHRPLPPEGQIQNGKILRTRVGDKFKYDLVLTVKLPDVEMIKPAALNGTIGIDVGFRKVGNSLLIGTVMSSDSAQKAVALEVPTMMVSALEHVDALRSELDDAASDLGKAITPLLKANPIDEEHDKYRLWRSLALRPLHVTLSFEQAYKLALWLKREPNLFPSEINEKVHTWWRSYSRKYRESHNRRKKQLSHRKHFYRETAAKLVAQNKLIVLEKIDLNVFAETKNKNTKLSNKARAQRFMASLGEFRDAIKNAADREGVPVIDVNPAYTSKTCSECGYLNKELKSEKEWNCPDCGVVHDRDENAANNLQKMGQKYLLDAAKTAAVVVK

SEQ ID NO: 24 - protein, 586 aa, Artificial Sequence
  Description of Artificial Sequence Synthetic polypeptide
MATRVYKYGLIPIGYPPKETIDELFKANVLWNNLVALHRKNREDWDDARRAASILYSDKIDELEKKEEDLDAAWKAFQQARMDEGTRDETNNKRLKSGRASINRLDAEKAEIYKELKPLRKEADKEIDKKQLNDAYRTKVNEAVSVRNSGIYSATAGQILENFKTARDRSFKESATTLRFHRFDGTGYYQFRCRRKGTNVDGISIDDFMSRNFEANPRCAVQSIDNSKKKPRIRIDAVLVGGQSKASKIHQEFDLIYHRPLPIDAQIQNGKILRTRVGDKFKYDLVLTLKIPDKEPISYNNLKGTIGIDIGFRRSVNSLLIGTVMSSDVTEKAYEIKVPPKIVEAFVHVIDLQSELDDAATDLGRIITPLLKAHPLDENHSKYKMWRSLALRPAHVTLSFEQAYKLAIWLKHEPDTFPEEITKQVHTWWRSYSRKYRELHNRRRNQLTHRKHFYREEAAKIVALNKLIVLEEINLTDFAETKEKNTKLSKKARAQRFMASLSEFRDAIRNAAQRDGIGIIDVNPAYTSKTCSECGNLNKGLRSEKQWSCPACGVVHDRDENAANNLQKMGQSYLESVKKETSEVIE

SEQ ID NO: 25 - protein, 595 aa, Artificial Sequence
  Description of Artificial Sequence Synthetic polypeptide
MAESTGFNMTTRVYKYGLIPIGYPPQAAIDELFRANKLWNTLVALHRESRENWDDARRSASILYSEKMDELDKKNDDVSEAFNGLNQARMEEGTKDETGNKRLLAERSIINMLKEDRAQIYAELNPLREKADKEIDKKALNDAYRKKCNEAVSAKVSGVYSSTAGQVYANFRTARDKAFKDNTTTKFHRFDGTGYFAFRCRSKDTSVNVDGISVEDFMSQGFVDYMRCAVMSVDESKKKPRIRINAVLTGGRTKASKVFQEFDWIYHRPLPPESQIQNGKILRTRVGDKFRYDLVLTVKLPDVEMVKPAALNGTIGIDVGFRKSGNTLLIGTVMSSDSAQKAVALEVPTMMVSALEHVVALQSELDDAASDLGKAITPLLKANPIDEEHSKYRLWRSLALRPMHVTLSFEQAYKLSLWLKHEPDLFPSEINEKVHTWWRSYSRKYREIHNRRKKQLTHRKHFYRETAAKLVAQNKLIVLEKIKLTDFAETKGKNTKLSNKARAQRFMASLAEFRDAIKNAADREGVPVIDVNPAYTSKTCSDCGYRNKELRSEKEWTCPECGVDHDRDENAANNLQKMGQKYLLDIEKAVSVVVE

SEQ ID NO: 26 - protein, 585 aa, Artificial Sequence
  Description of Artificial Sequence Synthetic polypeptide
MATRVYKYGLIPIGYPPKETIQELFRANVLWNNLVALHKKNREDWDDARRAASILYSDKIDELEKNKEDLDAVWKAFNQARMDEGTRDETNNKRLKSERASIDRLDAVRAEIYKELKPLRKEADKEIDKKQLNDSYRRKVNEAVSVRNSGIYNATAGQVLDNFKTARDRSFKENATLKFHKFDYTGYYHFRCRRKGAKVDGISVDDFMSRNFIANPRCAVQSIDNSKKKPRIRINAVLVGGQSKASKIHQEFDLIYHRPLPIDAQIQNGKILRTRVGDKFKYDLVLTLKIPDKEPISYNNLKGTIGIDIGFRRSVNTLLIGTVMSSDLTEKAYEIKVPPKIVEAFEHIIDLQSELDDAATDLGRIITPLLKAHPLDEDHSKYKMWRSLALRPAHVTLSFEQAYKLSIWLKHEPDTFPEEITKKVHTWWRSYSRKYRELHNRRRNQLTHRKHFYREEAAKIVALNKLIVLEEINLTDFAETKEKNTKLSKKARAQRFMASLSEFRDAIKNAAQRDGIGIINVNPAYTSKTCSECGNLNKDLRSEKQWSCPACGVVHDRDENAANNLQKMGQTYLESLKKETSEVIE

SEQ ID NO: 27 - protein, 585 aa, Artificial Sequence
  Description of Artificial Sequence Synthetic polypeptide
MATRVYKYGLIPIGYPPKETIQELFRANVLWNNLVALHKKNREDWDDARRAASILYSDKIDELEKNKEDLDAVWKAFNQARMDEGTRDETNNKRLKSERASIDRLDAVRAEIYKELKPLRKEADKEIDKKQLNDSYRRKVNEAVSVRNSGIYNATAGQVLDNFKTARDRSFKENATLKFHKFDYTGYYHFRCRRKGAKVDGISVDDFMSRNFIANPRCAVQSIDNSKKKPRIRINAVLVGGQSKASKIHQEFDLIYHRPLPIDAQIQNGKILRTRVGDKFKYDLVLTLKIPDKEPISYNNLKGTIGIDIGFRRSVNSLLIGTVMSSDVTEEAYEIIVPPKIVEAFEHVIDLQSELDDAATDLGRIITPLLKAHPLDEDHSKYKMWRSLALRPAHVTLSFEQAYKLAIWLKHEPDTFPEEITKKVHTWWRSYSRKYRELHNRRRNQLTHRKHFYREEAAKIVALNKLIVLEEINLTDFAETKEKNTKLSKKARAQRFMASLSEFRDAIKNAAQRDGIGIIDVNPAYTSKTCSECGNLNKDLRSEKQWSCPACGVVHDRDENAANNLQKMGQSYLENIKKETSEIIE

SEQ ID NO: 28 - protein, 586 aa, Artificial Sequence
  Description of Artificial Sequence Synthetic polypeptide
MATRVYKYGLIPIGYPPKETIDELFKANVLWNNLVALHRKNREDWDDARRAASILYSDKIDELEKKEEDLDAAWKAFQQARMDEGTRDETNNKRLKSGRASINRLDAEKAEIYKELKPLRKEADKEIDKKQLNDAYRTKVNEAVSVRNSGIYSATAGQILENFKTARDRSFKESATTLRFHRFDGTGYYQFRCRRKGTNVDGISIDDFMSRNFEANPRCAVQSIDNSKKKPRIRIDAVLVGGQSKASKIHQEFDLIYHRPLPIDAQIQNGKILRTRVGDKFKYDLVLTLKIPDKEPISYNNLKGTIGIDIGFRRSVNTLLIGTVMSSDVTEKAYEIKVPPKIVEAFVHVIDLQSELDDAATDLGRIITPLLKAHPLDENHSKYKMWRSLALRPAHVTLSFEQAYKLAIWLKHEPDTFPEEITMKVHTWWRSYSRKYRELHNRRRNQLTHRKHFYREEAAKIVALNKLIVLEEINLTDFAETKEKNTKLSKKARAQRFMASLSEFRDAIRNAAQRDGIGIIDVNPAYTSKTCSECGNLNKGLRSEKQWSCPACGVVHDRDENAANNLQKMGQSYLESVKKETSEVIE

SEQ ID NO: 29 - protein, 573 aa, Artificial Sequence
  Description of Artificial Sequence Synthetic polypeptide
MTIKVYKFGLLDPVSGWDQTAIDVLFLRNKLWNNLVAMEHDKRQAYRNLLLDSDTELAALQARLDAIEVEKASLITSKKALRAKARSRQVDTAEIDLEIKKLLEERKALGGQTKDLRERVKIEVKPLAAELDQQRYEKTKQLNKESGLWWCNSMTVIAAYEVGRLRAMREKNELRFHGFDGTGKYSVCRTGGFSLDHVMTGKLSFVSIRTLPIANLDDLSERGQRSRARHHLTMIVLRATTEEGTKIRHEVTWPIILHRPLPDDCLIKQIQVLRKRVGDRFEWTCSITVDTPEELKARLDSPSISVCGIDLGFRQVNNDLRVATLADSSGGLRYYTIGKDWLDSMDYVEAIQSDLSGTANSVWAQLRLILKELDEYPEALRERITDMLKAGAKTPIRAMRAMQKTLSNEPDLMPDALALLDDWKKRIRRRTKEMHDLRDKLINRRKDIYRNIACEIARDYSLVRIANLKLKDMVKLKRNDGTDTKLTDNARKNCNRAALSELTLYIQQACAKNGVALEKIDTTYMTRTCYQCGYLNPANTINLLLSCEGCGAEYDQDDNAAKNYLNATKPGTG

SEQ ID NO: 30 - protein, 596 aa, Artificial Sequence
  Description of Artificial Sequence Synthetic polypeptide
MKALVSILGMATRVYKYGLVPIGYPPEAVVGLYGELHKANSLWNTLVALHKESRENWDDARRSASINYSEKMDELDAKQQEISQAFDALRQVRMEEGTKDESNPKLKLERDIINRLKKEQGVIFAELKPLRKAADDNVDKKALNEAYRDKCKQAVSVKNCGIYRRTADQIYANFRTAREKAFKENATMRFHPFDGTGYFQFRCTRKGSNTDGITVDEFMKASFTENNRCAVQTVDDSKKKPRIRINSVLTGGPSRATKVFQEFDWIYHRPLPPDAQIQNGKILRTRVGDKFRYDLVLTIKVPDTELIKADKLNGTIGIDVGFRKSGDTILIGTVMSEDASQKAQEIKAPSKMVSALEHIIELQSELDDAATDLGKALTPLLLANPLPDDHPKFRMWQSLARRPAHVTLSFEKAYKFALWLNREPNVFPKEVTEKVHTWWRSYSRKYREIHNRRKKQLTHRKHFYRETAASIVAQKKLIVLEDINLTDFAETRDKNTKLSNKARAQRFLASLSEFRDAIKNAAEREGVKVIDVDPAYTSKTCSDCSFLNKALKSDKEWSCPNCGVVHDRDTNAANNLQKMGQVYIEEVKKEALKVIE

SEQ ID NO: 31 - protein, 627 aa, Artificial Sequence
  Description of Artificial Sequence Synthetic polypeptide
MGSRGLVKLVEPARLNFPEKNKKGGNAMISRVYKYGAVPLKKFPEVKFPREQFPEEGVEELRRANKLRNSLVWLHRKNNEKFEAARVAADAEYGEIAEKLDALEKTISQALTAKRQARAKAGTRDAKHPLVKAASETINELTKQRSDLWKALKPARIRADKRVDRKALTKQFDDAVKVVQHVKETGGLSSHCANEIVRYFKESRSRALNERATLRYRRFDGTGFWFYRFREPGVNKNGVDFDGLLTGNKTEARDNRNFVLTEKSRRGKRVIYKLRAKIAGGAKKDSKVYGHFDLILHRPIPENARIQSAKILRHRTGDKFTYTVSFTLKLPDVEQQTVEGSVLGLDIGFREMERNNSYRIATLATNDQSRRVETIDIARENRRGFLARMNHIDDLRSTMDENATELGKKLLPLLKTAKPLPDSHQQFIFTERLRKTRANVTLDFERSYKMARWFIRAPDEADFYGPEIVGMVLRWWEENSFKYREMHNLRRKALAERKEVYRMEAARLVGFGIPIAVEKLDMSKWAERKDSDNELSNRALSSRFLVAPSELIAAIENAAKREGVPFIKVNAANTSKACHACGTINKALKGELIWTCEECETKHDRDINAAINIAKRGILQAKKEKKQ

SEQ ID NO: 32 - protein, 586 aa, Artificial Sequence
  Description of Artificial Sequence Synthetic polypeptide
MATRVYKYGLIPIGYPAKETIDELFKANVLWNNLVALHRKNREDWDDARRAASVLYSDKIDDLEKKEEDLDAAWKAFQQARMDEGTRDETNNKRLKSERASINRLDTEKAEIYKELKPLRKEADKEIDKKQLNDAYRTKVNEAVSVRNSGIYSATAGQILENFKTARDRSFKESATTLRFHRFDGTGYYQFRCRRKGTNVDGISIDDFMSRNFEANPRCAVQSIDNSKKKPRIRIDAVLVGGQSKASKIHQEFDLIYHRPLPIDAQIQNGKILRTRVGDKFKYDLVLTLKIPDKEPISYNNLKGTIGIDIGFRRSVNSLLIGTVMSSDVTEEAYEIIVPPKIVEAFEHVIDLKSELDDAATDLGRIITPLMKAHPLDEDHSKYKMWRSLALRPAHVTLSFEQAYKLAIWLKHEPDTFPEEITKKVHTWWRSYSRKYRELHNRRRNQLTHRKHFYREEAAKIVALNKLIVLEEINLTDFAETKEKNTKLSKKARAQRFMASLSEFRDAIKNAAQRDGIGIIDVNPAYTSKTCSECGNLNKDLRSEKQWSCPACGVVHDRDENAANNLQKMGQTYLESLKKETSEVIE

SEQ ID NO: 33 - protein, 595 aa, Artificial Sequence
  Description of Artificial Sequence Synthetic polypeptide
MAESTGFNMTTRVYKYGLIPIGYPPQAAIDELFRANKLWNTLVALHRESRENWDDARRSASILYSEKMDELDKKNDDVSEAFNGLNQARMEEGTKDETGNKRLLAERSIINMLKEDRAQIYAELNPLREKADKEIDKKALNDAYRKKCNEAVSAKVSGVYSSTAGQVYANFRTARDKAFKDNTTTKFHRFDGTGYFAFRCRSKDTSVNVDGISVEDFMSQGFVDYMRCAVMSVDESKKKPRIRINAVLTGGRTKASKVFQEFDWIYHRPLPPESQIQNGKILRTRVGDKFRYDLVLTVKLPDVEMVKPAALNGTIGIDVGFRKSGNTLLIGTVMSSDSAQKAVALEVPKMMVSALEHVVALQSELDDAASDLGKAITPLLKANPIDEEHSKYRLWRSLALRPMHVTLSFEQAYKLSLWLKHEPDLFPSEINEKVHTWWRSYSRKYREIHNRRKKQLTHRKHFYRETAAKLVAQNKLIVLEKIKLTDFAETKGKNTKLSNKARAQRFMASLAEFRDAIKNAADREGVPVIDVNPAYTSKTCSDCGYRNKELRSEKEWTCPECGVDHDRDENAANNLQKMGQKYLLDIEKAVSVVVE

SEQ ID NO: 34 - protein, 587 aa, Artificial Sequence
  Description of Artificial Sequence Synthetic polypeptide
MTTRVYKYGLIPIGYPPQAAIDELFRANNLWNTLVALHRESRENWDDARRSASILYSEKMDELDKKNEDISEAFNGLNQARMDEGTKDETGNKRLQAERAIINRLKKERNEIYAELKPLRKEADKTVDRKALNDEYRKKCNTAVSAKVSGVYSGTAGELYAYFRTAKDKAFKENATLRFHRFDGTGYFAFRCRSKAVGVKVDGISVEDFMSKQFIDYLRCAVQSVDESRKKPRIRINAVLTGGRTTASKVFQEFDWIYHRPLPPEGQIQNGKILRTRVGDKFKHDLVLTVKLPDVEMIKPAALNGTIGIDVGFRKVGNSLLIGTVMYSDSAQKAEALEVPTMMVSALEHVVALQSELDNAASDLGKAITPLLKANPIDEEHDKYRLWRSLALRPLHVTLSFEQAYKLALWLKREPNLFPSEINEKVHTWWRSYSRKYREIHNRRKKQLTHRKHFYRETAAKLVAQNKLIVLEKINLTDFAETKNKNTKLSNKARAQRFMAALGEFRDAIKNAADREGVPVIDVNPAYTSKTCSECGYLNKELKSEKEWNCPDCGVVHDRDENAANNLQKMGQKYLLDAAKTAAVVVK

SEQ ID NO: 35 - protein, 604 aa, Artificial Sequence
  Description of Artificial Sequence Synthetic polypeptide
MAVEERTKKGVSVRVYKYGLVPKGCLPDEAKDELLRANNLWNKLVEISRKNQSDFDEVRKKAHPPYGEEMVRLETINEKIDDAYDRKRDARKEAGTRDETHPLIIKANAVIEILKEERSEIYDTLKLLRTEADKSVDKKALNESFKSNIKLARRETSLNSDTKEEIIRNDFRTARDRTFKTGGRLRFHTFDGTGYWHFRFRRNDEKGKKVDDYTIDDLFAGEKPSPKKPLPNRFKFLSRDDTRRKPRLRLRTTLAGGRTNASKVFQEFDVIYHRPVPEGAIIPNAKILRTRTGDKFRYDLVLTVYLPEPKHKDIPLDDAIGIDLGFREDPDDKYRKQVGAIISLDPSDEVEEIFAPPKMVKAFGHIDELKSVLDESAADLGMKIKPLMKDIRLPEDNEKYKQEYKLWNSIVNARANVTLSFEKAYKLALWCKKKDAGIPEDITGPVVYWWKGYSRRYRELHNLRKKQLLNRKDFYRQIASRLVLSGLLIGVENFNLSKIARSKDEDNVLNNKARANRFLLSPSEFRAAIKNAADREGIPCLEVNPANTSKICFDCGTLNKNLGSEKNWVCPACGCVHDRDTNAARNIAKRALEKYLEDRKSAGK

SEQ ID NO: 36 - protein, 594 aa, Artificial Sequence
  Description of Artificial Sequence Synthetic polypeptide
MYHFSMATRVYKYGLVPLGYPPDSVVGKGGELQRANNLWNTLVALHRESREHWDDARRSASILYSEKMDELDKKNEDINDAFDGLNKARMEEGTKDETGNKRLLAERAIINRLKKEQGDIYVELKPLRKEADKTVDRKALNDEYRNKCNTAVSAKVSGVYSRTAGELYAYFRTARDKAFKDNATLRFHRFDGTGYFAFRCRSKAVGVKVDGISVEDFMSQGFVDYMRCAVMSVDESKKKPRIRISAVLTGGATKASKVFQEFDWIYHRPLPPEGQIQNGKILRTRVGDKFKYDLVLTVKLPDVEMIEPAALNGTIGIDVGFRKVGNSLLIGTVMSSDSAQKAVVLEVPTMVVSALEHVDALRSELDDAASDLGKAITPLLKANPIDEEHDKYRLWRSLALRPLHVTLSFEQAYKLALWLKREPNLFPSEINEKVHTWWQSYSRKYRESHNRRKKQLSHRKHFYRETAAKLVAQNKLIVLEKIDLTVFAETKNKNTKLSNKARSQRYMAALGEFRDAIKNAADREGVPVIDVNPAYTSKTCSECGYLNKELKSEKEWNCPECGVVHDRDENAANNLQKMGQKYLSSRCKSSYGSS

SEQ ID NO: 37 - protein, 590 aa, Artificial Sequence
  Description of Artificial Sequence Synthetic polypeptide
MVSRQTDKDKTEVRVYKYGLIPVGYPPEETISELWRANNLWNTLVGLHYKHREKYEEARCEAHLLYGEVAERLAAIDEKIEQAYDDKRTARMKAGTRDAADPLIKEANAVIKALKDKRKAIYEELKPIRIEADKRVDKSALNSAFTKDINEATQSKNTGGLYSTTANEVKESFKTARDRSFKTNSKLRFHRFDGTGFYAFRFRRKDNKVDGVSFEELFQGNKETDQRFTFIGRDESRKKTRLRLRATLAGGARKASKIKHEFDLIYHRPIPDGSKIQNGKIMRTRIGDRFKYHLVLTIKQPKSEPLIVPKDVALGIDIGFRRIKDSIQVASITSSDPSLPSQNVMAPNKMIAGMERVIELQSILDDAASELGKLTKPILNDNPQLEDHKRFGLWKAMARYPNNVTLSFETAYKVARWLQSEADFIPPKAAKLVLEWWSAYSRRYRELHNLRAKQLLHRKHFYRQVASDLVAHRQLIVLEEINLSVFAEVKDRDNNLSDKARAQRFLAAPSEFRDAICNAAQREKVPFVTVPPQYTSKMCSSCSVINRELDSQKKWECPSCGVTHDRDENAARNIAKLGQKYFSGEKKKKK

SEQ ID NO: 38 - RNA, 37 nt, Artificial Sequence
  Description of Artificial Sequence Synthetic oligonucleotide
guuuuagaacccuguugagugggcauaaacucaaacu

SEQ ID NO: 39 - RNA, 37 nt, Artificial Sequence
  Description of Artificial Sequence Synthetic oligonucleotide
aguuugaguuuaugcccacucaacaggguucuaaaac

SEQ ID NO: 40 - RNA, 36 nt, Artificial Sequence
  Description of Artificial Sequence Synthetic oligonucleotide
guauaacgacccugcgaagugggguguaacuucgac

SEQ ID NO: 41 - RNA, 36 nt, Artificial Sequence
  Description of Artificial Sequence Synthetic oligonucleotide
gucgaaguuacaccccacuucgcagggucguuauac

SEQ ID NO: 42 - RNA, 26 nt, Artificial Sequence
  Description of Artificial Sequence Synthetic oligonucleotide
gauuaaggccccuguguaguggggug

SEQ ID NO: 43 - RNA, 26 nt, Artificial Sequence
  Description of Artificial Sequence Synthetic oligonucleotide
caccccacuacacaggggccuuaauc

SEQ ID NO: 44 - RNA, 36 nt, Artificial Sequence
  Description of Artificial Sequence Synthetic oligonucleotide
ccuauagaacccugccaaguggguagugacuuggac

SEQ ID NO: 45 - RNA, 36 nt, Artificial Sequence
  Description of Artificial Sequence Synthetic oligonucleotide
guccaagucacuacccacuuggcaggguucuauagg

SEQ ID NO: 46 - RNA, 36 nt, Artificial Sequence; identical to SEQ ID NO: 40
  Description of Artificial Sequence Synthetic oligonucleotide

SEQ ID NO: 47 - RNA, 36 nt, Artificial Sequence; identical to SEQ ID NO: 41
  Description of Artificial Sequence Synthetic oligonucleotide

SEQ ID NO: 48 - RNA, 36 nt, Artificial Sequence
  Description of Artificial Sequence Synthetic oligonucleotide
guguaaggccccugugcauugggguguaaaugcaac

SEQ ID NO: 49 - RNA, 36 nt, Artificial Sequence
  Description of Artificial Sequence Synthetic oligonucleotide
guugcauuuacaccccaaugcacaggggccuuacac

SEQ ID NO: 50 - RNA, 36 nt, Artificial Sequence
  Description of Artificial Sequence Synthetic oligonucleotide
ccuuaaggccccugucuagugggguguaacuagaac

SEQ ID NO: 51 - RNA, 36 nt, Artificial Sequence
  Description of Artificial Sequence Synthetic oligonucleotide
guucuaguuacaccccacuagacaggggccuuaagg

SEQ ID NO: 52 - RNA, 34 nt, Artificial Sequence
  Description of Artificial Sequence Synthetic oligonucleotide
aguagaacccugucgcuugggugguaaagcgaac

SEQ ID NO: 53 - RNA, 34 nt, Artificial Sequence
  Description of Artificial Sequence Synthetic oligonucleotide
guucgcuuuaccacccaagcgacaggguucuacu

SEQ ID NO: 54 - RNA, 29 nt, Artificial Sequence
  Description of Artificial Sequence Synthetic oligonucleotide
auuguaagacccuguuggaugggguguga

SEQ ID NO: 55 - RNA, 29 nt, Artificial Sequence
  Description of Artificial Sequence Synthetic oligonucleotide
ucacaccccauccaacagggucuuacaau

SEQ ID NO: 56 - RNA, 36 nt, Artificial Sequence
  Description of Artificial Sequence Synthetic oligonucleotide
cuuuaaggacccuguacguugggguguaaacguaac

SEQ ID NO: 57 - RNA, 36 nt, Artificial Sequence
  Description of Artificial Sequence Synthetic oligonucleotide
guuacguuuacaccccaacguacaggguccuuaaag

SEQ ID NO: 58 - RNA, 35 nt, Artificial Sequence
  Description of Artificial Sequence Synthetic oligonucleotide
uuuaaggacccuguacguugggguguaaacguaac

SEQ ID NO: 59 - RNA, 35 nt, Artificial Sequence
  Description of Artificial Sequence Synthetic oligonucleotide
guuacguuuacaccccaacguacaggguccuuaaa

SEQ ID NO: 60 - RNA, 27 nt, Artificial Sequence
  Description of Artificial Sequence Synthetic oligonucleotide
gugauagugccugcucaguggcuuagu

SEQ ID NO: 61 - RNA, 27 nt, Artificial Sequence
  Description of Artificial Sequence Synthetic oligonucleotide
acuaagccacugagcaggcacuaucac

SEQ ID NO: 62 - RNA, 36 nt, Artificial Sequence
  Description of Artificial Sequence Synthetic oligonucleotide
gcuugagaacccugucgauuggguggugaaucgaac

SEQ ID NO: 63 - RNA, 36 nt, Artificial Sequence
  Description of Artificial Sequence Synthetic oligonucleotide
guucgauucaccacccaaucgacaggguucucaagc

SEQ ID NO: 64 - RNA, 36 nt, Artificial Sequence; identical to SEQ ID NO: 40
  Description of Artificial Sequence Synthetic oligonucleotide

SEQ ID NO: 65 - RNA, 36 nt, Artificial Sequence; identical to SEQ ID NO: 41
  Description of Artificial Sequence Synthetic oligonucleotide

SEQ ID NO: 66 - RNA, 36 nt, Artificial Sequence
  Description of Artificial Sequence Synthetic oligonucleotide
guugcauuuacaccccaaugcacaggggccuuagac

SEQ ID NO: 67 - RNA, 36 nt, Artificial Sequence
  Description of Artificial Sequence Synthetic oligonucleotide
guuauaggacccugccggguggggugugacccggac

SEQ ID NO: 68 - RNA, 36 nt, Artificial Sequence
  Description of Artificial Sequence Synthetic oligonucleotide
guccgggucacaccccacccggcaggguccuauaac

SEQ ID NO: 69 - RNA, 36 nt, Artificial Sequence; identical to SEQ ID NO: 66
  Description of Artificial Sequence Synthetic oligonucleotide

SEQ ID NO: 70 - RNA, 36 nt, Artificial Sequence
  Description of Artificial Sequence Synthetic oligonucleotide
gucuaaggccccugugcauugggguguaaaugcaac

SEQ ID NO: 71 - RNA, 36 nt, Artificial Sequence
  Description of Artificial Sequence Synthetic oligonucleotide
auuuaaugacccugcguguuggggugugaacacgac

SEQ ID NO: 72 - RNA, 36 nt, Artificial Sequence
  Description of Artificial Sequence Synthetic oligonucleotide
gucguguucacaccccaacacgcagggucauuaaau

SEQ ID NO: 73 - RNA, 39 nt, Artificial Sequence
  Description of Artificial Sequence Synthetic oligonucleotide
ucagguaucucgacccuacggauggggggaacauccggc

SEQ ID NO: 74 - RNA, 39 nt, Artificial Sequence
  Description of Artificial Sequence Synthetic oligonucleotide
gccggauguuccccccauccguagggucgagauaccuga

SEQ ID NO: 75 - RNA, 36 nt, Artificial Sequence; identical to SEQ ID NO: 56
  Description of Artificial Sequence Synthetic oligonucleotide

SEQ ID NO: 76 - RNA, 36 nt, Artificial Sequence; identical to SEQ ID NO: 57
  Description of Artificial Sequence Synthetic oligonucleotide

SEQ ID NO: 77 - RNA, 36 nt, Artificial Sequence; identical to SEQ ID NO: 40
  Description of Artificial Sequence Synthetic oligonucleotide

SEQ ID NO: 78 - RNA, 36 nt, Artificial Sequence; identical to SEQ ID NO: 41
  Description of Artificial Sequence Synthetic oligonucleotide

SEQ ID NO: 79 - RNA, 30 nt, Artificial Sequence
  Description of Artificial Sequence Synthetic oligonucleotide
cuuuaaggacccuguacguugggguguaaa

SEQ ID NO: 80 - RNA, 30 nt, Artificial Sequence
  Description of Artificial Sequence Synthetic oligonucleotide
uuuacaccccaacguacaggguccuuaaag

SEQ ID NO: 81 - RNA, 37 nt, Artificial Sequence
  Description of Artificial Sequence Synthetic oligonucleotide
guugcauuuacaccccaaugcacaggggccuuagaca

SEQ ID NO: 82 - RNA, 37 nt, Artificial Sequence
  Description of Artificial Sequence Synthetic oligonucleotide
ugucuaaggccccugugcauugggguguaaaugcaac

SEQ ID NO: 83 - RNA, 36 nt, Artificial Sequence; identical to SEQ ID NO: 56
  Description of Artificial Sequence Synthetic oligonucleotide

SEQ ID NO: 84 - RNA, 36 nt, Artificial Sequence; identical to SEQ ID NO: 57
  Description of Artificial Sequence Synthetic oligonucleotide

SEQ ID NO: 85 - RNA, 24 nt, Artificial Sequence
  Description of Artificial Sequence Synthetic oligonucleotide
ggccccugugcauugggguguaaa

SEQ ID NO: 86 - RNA, 24 nt, Artificial Sequence
  Description of Artificial Sequence Synthetic oligonucleotide
uuuacaccccaaugcacaggggcc

SEQ ID NO: 87 - RNA, 30 nt, Artificial Sequence; identical to SEQ ID NO: 79
  Description of Artificial Sequence Synthetic oligonucleotide

SEQ ID NO: 88 - RNA, 30 nt, Artificial Sequence; identical to SEQ ID NO: 80
  Description of Artificial Sequence Synthetic oligonucleotide

SEQ ID NO: 89 - RNA, 36 nt, Artificial Sequence; identical to SEQ ID NO: 56
  Description of Artificial Sequence Synthetic oligonucleotide

SEQ ID NO: 90 - RNA, 36 nt, Artificial Sequence; identical to SEQ ID NO: 57
  Description of Artificial Sequence Synthetic oligonucleotide

SEQ ID NO: 91 - RNA, 36 nt, Artificial Sequence; identical to SEQ ID NO: 56
  Description of Artificial Sequence Synthetic oligonucleotide

SEQ ID NO: 92 - RNA, 36 nt, Artificial Sequence; identical to SEQ ID NO: 57
  Description of Artificial Sequence Synthetic oligonucleotide

SEQ ID NO: 93 - RNA, 29 nt, Artificial Sequence
  Description of Artificial Sequence Synthetic oligonucleotide
gagcgguuaacagggugucgauauagauu

SEQ ID NO: 94 - RNA, 29 nt, Artificial Sequence
  Description of Artificial Sequence Synthetic oligonucleotide
aaucuauaucgacacccuguuaaccgcuc

SEQ ID NO: 95 - RNA, 36 nt, Artificial Sequence
  Description of Artificial Sequence Synthetic oligonucleotide
gauuaaggccccuguguagugggguguaacuacaac

SEQ ID NO: 96 - RNA, 36 nt, Artificial Sequence
  Description of Artificial Sequence Synthetic oligonucleotide
guuguaguuacaccccacuacacaggggccuuaauc

SEQ ID NO: 97 - RNA, 36 nt, Artificial Sequence; identical to SEQ ID NO: 71
  Description of Artificial Sequence Synthetic oligonucleotide

SEQ ID NO: 98 - RNA, 36 nt, Artificial Sequence; identical to SEQ ID NO: 72
  Description of Artificial Sequence Synthetic oligonucleotide

SEQ ID NO: 99 - RNA, 33 nt, Artificial Sequence
  Description of Artificial Sequence Synthetic oligonucleotide
uaaggacccuguacguugggguguaaacguaac

SEQ ID NO: 100 - RNA, 33 nt, Artificial Sequence
  Description of Artificial Sequence Synthetic oligonucleotide
guuacguuuacaccccaacguacaggguccuua

SEQ ID NO: 101 - RNA, 24 nt, Artificial Sequence
  Description of Artificial Sequence Synthetic oligonucleotide
ggccccugugcauugggguguaaa

SEQ ID NO: 102 - RNA, 24 nt, Artificial Sequence
  Description of Artificial Sequence Synthetic oligonucleotide
uuuacaccccaaugcacaggggcc

SEQ ID NO: 103 - RNA, 36 nt, Artificial Sequence; identical to SEQ ID NO: 66
  Description of Artificial Sequence Synthetic oligonucleotide

SEQ ID NO: 104 - RNA, 36 nt, Artificial Sequence; identical to SEQ ID NO: 70
  Description of Artificial Sequence Synthetic oligonucleotide

SEQ ID NO: 105 - RNA, 36 nt, Artificial Sequence; identical to SEQ ID NO: 40
  Description of Artificial Sequence Synthetic oligonucleotide

SEQ ID NO: 106 - RNA, 36 nt, Artificial Sequence; identical to SEQ ID NO: 41
  Description of Artificial Sequence Synthetic oligonucleotide

SEQ ID NO: 107 - RNA, 36 nt, Artificial Sequence; identical to SEQ ID NO: 66
  Description of Artificial Sequence Synthetic oligonucleotide

SEQ ID NO: 108 - RNA, 36 nt, Artificial Sequence; identical to SEQ ID NO: 70
  Description of Artificial Sequence Synthetic oligonucleotide

SEQ ID NO: 109 - RNA, 36 nt, Artificial Sequence; identical to SEQ ID NO: 50
  Description of Artificial Sequence Synthetic oligonucleotide

SEQ ID NO: 110 - RNA, 36 nt, Artificial Sequence; identical to SEQ ID NO: 51
  Description of Artificial Sequence Synthetic oligonucleotide

SEQ ID NO: 111 - RNA, 34 nt, Artificial Sequence
  Description of Artificial Sequence Synthetic oligonucleotide
auaacgacccugcgaagugggguguaacuucgac

SEQ ID NO: 112 - RNA, 34 nt, Artificial Sequence; identical to SEQ ID NO: 111
  Description of Artificial Sequence Synthetic oligonucleotide

SEQ ID NO: 113 - RNA, 34 nt, Artificial Sequence
  Description of Artificial Sequence Synthetic oligonucleotide
uuaaggccccugucuagugggguguaacuagaac

SEQ ID NO: 114 - RNA, 35 nt, Artificial Sequence
  Description of Artificial Sequence Synthetic oligonucleotide
cuugagaacccugucgauuggguggugaaucgaac

SEQ ID NO: 115 - RNA, 34 nt, Artificial Sequence; identical to SEQ ID NO: 111
  Description of Artificial Sequence Synthetic oligonucleotide

SEQ ID NO: 116 - RNA, 33 nt, Artificial Sequence
  Description of Artificial Sequence Synthetic oligonucleotide
auaggacccugccggguggggugugacccggac

SEQ ID NO: 117 - RNA, 35 nt, Artificial Sequence
  Description of Artificial Sequence Synthetic oligonucleotide
uuuaaugacccugcguguuggggugugaacacgac

SEQ ID NO: 118 - RNA, 34 nt, Artificial Sequence
  Description of Artificial Sequence Synthetic oligonucleotide
uaucucgacccuacggauggggggaacauccggc

SEQ ID NO: 119 - RNA, 34 nt, Artificial Sequence; identical to SEQ ID NO: 111
  Description of Artificial Sequence Synthetic oligonucleotide

SEQ ID NO: 120 - RNA, 35 nt, Artificial Sequence; identical to SEQ ID NO: 58
  Description of Artificial Sequence Synthetic oligonucleotide

SEQ ID NO: 121 - RNA, 35 nt, Artificial Sequence; identical to SEQ ID NO: 58
  Description of Artificial Sequence Synthetic oligonucleotide

SEQ ID NO: 122 - RNA, 35 nt, Artificial Sequence; identical to SEQ ID NO: 58
  Description of Artificial Sequence Synthetic oligonucleotide

SEQ ID NO: 123 - RNA, 35 nt, Artificial Sequence; identical to SEQ ID NO: 117
  Description of Artificial Sequence Synthetic oligonucleotide

SEQ ID NO: 124 - RNA, 34 nt, Artificial Sequence; identical to SEQ ID NO: 111
  Description of Artificial Sequence Synthetic oligonucleotide

SEQ ID NO: 125 - RNA, 34 nt, Artificial Sequence; identical to SEQ ID NO: 113
  Description of Artificial Sequence Synthetic oligonucleotide

SEQ ID NO: 126 - RNA, 33 nt, Artificial Sequence
  Description of Artificial Sequence Synthetic oligonucleotide
uaaggccccugucuagugggguguaacuagaac

SEQ ID NO: 127 - RNA, 85 nt, Artificial Sequence
  Description of Artificial Sequence Synthetic oligonucleotide
uuuggaaagcguacuggcugaauuuauaagccagaacgaaauucagugcgaugcacaggcgauuaaaucauuuguaguuggauua

SEQ ID NO: 128 - RNA, 45 nt, Artificial Sequence
  Description of Artificial Sequence Synthetic oligonucleotide
auucagugcgaugcacaggcgauuaaaucauuuguaguuggauua

SEQ ID NO: 129 - RNA, 49 nt, Artificial Sequence
  Description of Artificial Sequence Synthetic oligonucleotide
auuucccguaucaggaaaggucguucaguucucacguuugcacuggaac

SEQ ID NO: 130 - RNA, 102 nt, Artificial Sequence
  Description of Artificial Sequence Synthetic polynucleotide
acuauuauaaaauuuugaagucauuuaaaaaugagaaccagaguuuacaaauacggucuuguuccuaucggcuauccaccacaggaagcuguagaugaacuu

SEQ ID NO: 131 - RNA, 91 nt, Artificial Sequence
  Description of Artificial Sequence Synthetic oligonucleotide
auuuugaagucauuuaaaaaugagaaccagaguuuacaaauacggucuuguuccuaucggcuauccaccacaggaagcuguagaugaacuu

SEQ ID NO: 132 - RNA, 91 nt, Artificial Sequence
  Description of Artificial Sequence Synthetic oligonucleotide
cucuggaacaaacuuguugagauuucaagaaaaggaacguugaauaaaaaucugggaagcgaaaaaaacugggucugcccugccugugggu

SEQ ID NO: 133 - RNA, 76 nt, Artificial Sequence
  Description of Artificial Sequence Synthetic oligonucleotide
ugauacgggaaauucuguuaguuuaauaugaaauugugccuuuggguauaaauccugcgcggccgguuucuuuacc

SEQ ID NO: 134 - RNA, 40 nt, Artificial Sequence
  Description of Artificial Sequence Synthetic oligonucleotide
cugggaagcgaaaaaaacugggucugcccugccugugggu

SEQ ID NO: 135 - RNA, 127 nt, Artificial Sequence
  Description of Artificial Sequence Synthetic polynucleotide
auuugaguuugcggaauaguuuaagcguucuccacggggaugccucuuuuggcacugaaaacccuugcuguguaagcaguaaggcugauccauuggggagaacugccauggcuguugaggaaagaac

SEQ ID NO: 136 - RNA, 49 nt, Artificial Sequence; identical to SEQ ID NO: 129
  Description of Artificial Sequence Synthetic oligonucleotide

SEQ ID NO: 137 - RNA, 55 nt, Artificial Sequence
  Description of Artificial Sequence Synthetic oligonucleotide
uccugaagcgaauguacaaacuuuaaggcugggcucuaguggcucagccucuuuu

SEQ ID NO: 138 - RNA, 72 nt, Artificial Sequence
  Description of Artificial Sequence Synthetic oligonucleotide
auauagcuaaaaguggcccaaacagcgcuaucauccuggcucagguuaacgacgaagagcguuuaacugaac

SEQ ID NO: 139 - RNA, 84 nt, Artificial Sequence
  Description of Artificial Sequence Synthetic oligonucleotide
uuacggcguaaagagaguaacauuucuuuuccugaagcgaauguacaaacuuuaaggcugggcucuaguggcucagccucuuuu

SEQ ID NO: 140 - RNA, 107 nt, Artificial Sequence
  Description of Artificial Sequence Synthetic polynucleotide
auaaauacggacuaaucccuguuggguauccgccugaagaaaccauuuccgagcuauggcgcgccaauaaucuuuggaacacguuaguuggccuccacuauaaaugu

SEQ ID NO: 141 - RNA, 148 nt, Artificial Sequence
  Description of Artificial Sequence Synthetic polynucleotide
auaaggggaggaucagaugaccauuuggcuaacucuccugccccgcgaccaaauaauuagacacgcagaaagccagcauuuugguuagucgacaaacagauaaagauaaaacagagguccguguuuauaaauacggacuaaucccugu

SEQ ID NO: 142 - RNA, 63 nt, Artificial Sequence
  Description of Artificial Sequence Synthetic oligonucleotide
augacaaaagaaauuaagggaauaaaaaaugaucaauugcuauaaauucggaugcuugcagcc

SEQ ID NO: 143 - RNA, 51 nt, Artificial Sequence
  Description of Artificial Sequence Synthetic oligonucleotide
auuaagggaauaaaaaaugaucaauugcuauaaauucggaugcuugcagcc

SEQ ID NO: 144 - RNA, 85 nt, Artificial Sequence
  Description of Artificial Sequence Synthetic oligonucleotide
guugggggcggaaccaaacuggacuuguccaaguuguaaagcagagcaugacagggaucacaaugcagcaauaaauauugcaagg

SEQ ID NO: 145 - RNA, 52 nt, Artificial Sequence
  Description of Artificial Sequence Synthetic oligonucleotide
auuuagacgagucgaugcgcauguguuuuguguuacucaaaagacccuguac

SEQ ID NO: 146 - RNA, 149 nt, Artificial Sequence
  Description of Artificial Sequence Synthetic polynucleotide
ugcaguaauuguccuucugugugguguuaaauaaauauggcuacuagagcauacaaauacggcuuaauuccuaucgguuaucccccaaaggaaaccauugaugagcuuuuuaaggcaaacguauuauggaauaaccuaguagccuuaca

SEQ ID NO: 147 - RNA, 155 nt, Artificial Sequence
  Description of Artificial Sequence Synthetic polynucleotide
auauuugcaguaauuguccuucugugugguguuaaauaaauauggcuacuagagcauacaaauacggcuuaauuccuaucgguuaucccccaaaggaaaccauugaugagcuuuuuaaggcaaacguauuauggaauaaccuaguagccuuacac

SEQ ID NO: 148 - RNA, 122 nt, Artificial Sequence
  Description of Artificial Sequence Synthetic polynucleotide
aggcuguagaaucguaguauagaguggaggcucgucugauucagaauuacggugacguauuuucguuaggcugccggagccaggcguuuggcaucgagcuuggcgaggagagccucuaaauc

SEQ ID NO: 149 - RNA, 49 nt, Artificial Sequence; identical to SEQ ID NO: 129
  Description of Artificial Sequence Synthetic oligonucleotide

SEQ ID NO: 150 - RNA, 87 nt, Artificial Sequence
  Description of Artificial Sequence Synthetic oligonucleotide
ucagcuguagcguaaagagaguaacauuucuucuccugacuuacuguacaaacuuuaaggcugggcuguaauggcucagccucuuuu

SEQ ID NO: 151 - RNA, 54 nt, Artificial Sequence
  Description of Artificial Sequence Synthetic oligonucleotide
uccugacuuacuguacaaacuuuaaggcugggcuguaauggcucagccucuuuu

SEQ ID NO: 152 - RNA, 71 nt, Artificial Sequence
  Description of Artificial Sequence Synthetic oligonucleotide
aagggaaaaggugguuugaaauuccuauacguccgggguucuccucaugucaucggaggagcgaauugauc

SEQ ID NO: 153 - RNA, 87 nt, Artificial Sequence; identical to SEQ ID NO: 150
  Description of Artificial Sequence Synthetic oligonucleotide

SEQ ID NO: 154 - RNA, 116 nt, Artificial Sequence
  Description of Artificial Sequence Synthetic polynucleotide
auugugagagaagaaagugauccaccugcucagcuguagcguaaagagaguaacauuucuucuccugacuuacuguacaaacuuuaaggcugggcuguaauggcucagccucuuuu

SEQ ID NO: 155 - RNA, 40 nt, Artificial Sequence
  Description of Artificial Sequence Synthetic oligonucleotide
guaucuugagaaaagcuggggaaugccagaacgcauuuuu

SEQ ID NO: 156 - RNA, 99 nt, Artificial Sequence
  Description of Artificial Sequence Synthetic oligonucleotide
aggcuccgugaacgggcuugcgggagcuuagaaaacuccggauguaagccgcaauuuguuuugcguauguggcuugaguuagcgcucaagccuuuuuua

SEQ ID NO: 157 - RNA, 61 nt, Artificial Sequence
  Description of Artificial Sequence Synthetic oligonucleotide
gaccaagaccagaacgcagcuaaaaauuuccugcgaggucaggaguuuuaucuugccgaac

SEQ ID NO: 158 - RNA, 65 nt, Artificial Sequence
  Description of Artificial Sequence Synthetic oligonucleotide
gaccaagaccagaacgcagcuaaaaauuuccugcgaggucaggaguuuuaucuugccgaacagau

SEQ ID NO: 159 - RNA, 61 nt, Artificial Sequence
  Description of Artificial Sequence Synthetic oligonucleotide
accggcauggacaagagagacauucauuuccgcugcgagaaaugcgauacucugcacgacc

SEQ ID NO: 160 - RNA, 38 nt, Artificial Sequence
  Description of Artificial Sequence Synthetic oligonucleotide
cucuggcuucgaccaggcugcgcuggacgugcuguacc

SEQ ID NO: 161 - RNA, 35 nt, Artificial Sequence
  Description of Artificial Sequence Synthetic oligonucleotide
ccaaucuuaugaccaguacuuaaaagacccuguac

SEQ ID NO: 162 - RNA, 49 nt, Artificial Sequence
  Description of Artificial Sequence Synthetic oligonucleotide
auaauugaaagaacuucacccauuguuucuucuaccauugagugaccag

SEQ ID NO: 163 - RNA, 43 nt, Artificial Sequence
  Description of Artificial Sequence Synthetic oligonucleotide
auaauugaaagaacuucacccauuguuucuucuaccauugagu

SEQ ID NO: 164 - RNA, 81 nt, Artificial Sequence
  Description of Artificial Sequence Synthetic oligonucleotide
ugccggaugaagcgaaagaugagcuuuuaagggcgaacaaucucuggaacaaacuuguugagauuucaagaaaaggaacgu

SEQ ID NO: 165 - RNA, 107 nt, Artificial Sequence
  Description of Artificial Sequence Synthetic polynucleotide
auacgggcuggugccgaaggggugccugccggaugaagcgaaagaugagcuuuuaagggcgaacaaucucuggaacaaacuuguugagauuucaagaaaaggaacgu

SEQ ID NO: 166 - RNA, 68 nt, Artificial Sequence
  Description of Artificial Sequence Synthetic oligonucleotide
gcaauauagcaauuaaauaaagauagcuguuaaaccagagccuuauagauugcaccucuugaacaguu

SEQ ID NO: 167 - RNA, 96 nt, Artificial Sequence
  Description of Artificial Sequence Synthetic oligonucleotide
gauccaacugcucauuugcagcguaaagagaguaacauuucuucuccugacuuacuguaaaaacuuaaggcugggcuguaauggcucagccucauu

SEQ ID NO: 168 - RNA, 90 nt, Artificial Sequence
  Description of Artificial Sequence Synthetic oligonucleotide
acugcucauuugcagcguaaagagaguaacauuucuucuccugacuuacuguaaaaacuuaaggcugggcuguaauggcucagccucauu

SEQ ID NO: 169 - RNA, 53 nt, Artificial Sequence
  Description of Artificial Sequence Synthetic oligonucleotide
gcugcaaaugagcaguuggaucacuuugaguuuaacguggucuguagcccgau

SEQ ID NO: 170 - RNA, 68 nt, Artificial Sequence; identical to SEQ ID NO: 166
  Description of Artificial Sequence Synthetic oligonucleotide

SEQ ID NO: 171 - RNA, 175 nt, Artificial Sequence
  Description of Artificial Sequence Synthetic polynucleotide
gugugaaauaaauauggcuacuagaguauauaaauauggcuuaauuccuaucgguuauccuccaaaggaaaccauucaagagcuauuuagagcaaacguauuauggaauaaccuaguagccuugcacaaaaaaaacagggaagauugggacgaugcucgaagaaacaaagaccuu

SEQ ID NO: 172 - RNA, 56 nt, Artificial Sequence
  Description of Artificial Sequence Synthetic oligonucleotide
gaugcucgaagaaacaaagaccuuagaucugaaaaacaauggucaugccccgcaug

SEQ ID NO: 173 - RNA, 126 nt, Artificial Sequence
  Description of Artificial Sequence Synthetic polynucleotide
auaaaagcaauguaaauacuauugaucagcuaaguaguuccacaacuguuaagauccuguuuuuuagaguuuugcuuagauuugaucauggucuauuugccauacaauguuuggucguuuaacccu

SEQ ID NO: 174 - RNA, 128 nt, Artificial Sequence
  Description of Artificial Sequence Synthetic polynucleotide
auauaaaagcaauguaaauacuauugaucagcuaaguaguuccacaacuguuaagauccuguuuuuuagaguuuugcuuagauuugaucauggucuauuugccauacaauguuuggucguuuaacccu

SEQ ID NO: 175 - RNA, 52 nt, Artificial Sequence
  Description of Artificial Sequence Synthetic oligonucleotide
aacaaaggccuuagaucugaaaaacaauggucaugcccugcauguggaguug

SEQ ID NO: 176 - RNA, 46 nt, Artificial Sequence
  Description of Artificial Sequence Synthetic oligonucleotide
ugucccugcugguuggguaaaaaccagacugauugaguugaugauu

SEQ ID NO: 177 - RNA, 68 nt, Artificial Sequence
  Description of Artificial Sequence Synthetic oligonucleotide
uauaaauuuacgcccaagcagggauuuauuuugaggauuuuuaagaacaaagcauuugcgcguuuugc

SEQ ID NO: 178 - RNA, 34 nt, Artificial Sequence
  Description of Artificial Sequence Synthetic oligonucleotide
aaagaguggucauguccaaacuguggugugguac

SEQ ID NO: 179 - RNA, 32 nt, Artificial Sequence
  Description of Artificial Sequence Synthetic oligonucleotide
caaacuguggugugguacaugauagggauacc

SEQ ID NO: 180 - RNA, 40 nt, Artificial Sequence; identical to SEQ ID NO: 155
  Description of Artificial Sequence Synthetic oligonucleotide

SEQ ID NO: 181 - RNA, 98 nt, Artificial Sequence
  Description of Artificial Sequence Synthetic oligonucleotide
aggcuccgugaacgggcuugcgggagcuuagaaaacuccggauguaagccgcaauuuguuuugcguauguggcuugaguuagcgcucaagccuuuuuu

SEQ ID NO: 182 - RNA, 97 nt, Artificial Sequence
  Description of Artificial Sequence Synthetic oligonucleotide
aggcuccgugaacgggcuugcgggagcuuagaaaacuccggauguaagccgcaauuuguuuugcguauguggcuugaguuagcgcucaagccuuuuu

SEQ ID NO: 183 - RNA, 68 nt, Artificial Sequence; identical to SEQ ID NO: 166
  Description of Artificial Sequence Synthetic oligonucleotide

SEQ ID NO: 184 - RNA, 51 nt, Artificial Sequence
  Description of Artificial Sequence Synthetic oligonucleotide
acuugaugagucgaugcgcauguguuuuguguuacucaaaagacccuguac

SEQ ID NO: 185 - RNA, 157 nt, Artificial Sequence
  Description of Artificial Sequence Synthetic polynucleotide
uuugaaaaaagagacaucugagguuauagaaugauaaauacgauccuauauucuccaacucaaaaagcgacuuuaucuaaugauuacaauaagcuaugcucacaucacuugaugagucgaugcgcauguguuuuguguuacucaaaagacccuguac

SEQ ID NO: 186 - RNA, 82 nt, Artificial Sequence
  Description of Artificial Sequence Synthetic oligonucleotide
uaacgauuucucuggcacguauaccuagccguuuacguaacaauagaguugccuugaucauuaaguuacuauaucucuggag

SEQ ID NO: 187 - RNA, 81 nt, Artificial Sequence
  Description of Artificial Sequence Synthetic oligonucleotide
aacgauuucucuggcacguauaccuagccguuuacguaacaauagaguugccuugaucauuaaguuacuauaucucuggag

SEQ ID NO: 188 - RNA, 72 nt, Artificial Sequence
  Description of Artificial Sequence Synthetic oligonucleotide
cucacuagcuaaucauuugugguuggucuguuuggucgugccuuuguugacugcugcuauugcucacaaaca

SEQ ID NO: 189 - RNA, 55 nt, Artificial Sequence; identical to SEQ ID NO: 137
  Description of Artificial Sequence Synthetic oligonucleotide

SEQ ID NO: 190 - RNA, 84 nt, Artificial Sequence
  Description of Artificial Sequence Synthetic oligonucleotide
uuacggcguaaauagaguaacauuucuuuuccugaagcgaauguacaaacuuuaaggcugggcucuaguggcucagccucuuuu

SEQ ID NO: 191 - RNA, 53 nt, Artificial Sequence
  Description of Artificial Sequence Synthetic oligonucleotide
cugggcgccucccuuccucaugaaggggguacaacgacccugcgagguggguu

SEQ ID NO: 192 - RNA, 107 nt, Artificial Sequence; identical to SEQ ID NO: 165
  Description of Artificial Sequence Synthetic polynucleotide

SEQ ID NO: 193 - RNA, 36 nt, Artificial Sequence
  Description of Artificial Sequence Synthetic oligonucleotide
aaauuuucuuucgccggggcugguaaaucucuugcc

SEQ ID NO: 194 - RNA, 84 nt, Artificial Sequence
  Description of Artificial Sequence Synthetic oligonucleotide
uuuuagcuguccgcucuggaaaaccucguguguguaacuguacauguacacauauccccucaaagccaaggcccucgccuguac

SEQ ID NO: 195 - RNA, 32 nt, Artificial Sequence
  Description of Artificial Sequence Synthetic oligonucleotide
acuaaaaggaaugcuuugagguaauagccaag

SEQ ID NO: 196 - RNA, 96 nt, Artificial Sequence
  Description of Artificial Sequence Synthetic oligonucleotide
auguuugcaaacauguacagcacguuagcaacaaaaagagagcgggcaauaaagcucacaacgcaacucuaucugcuuguuuagcucagcaaggcu

SEQ ID NO: 197 - RNA, 72 nt, Artificial Sequence
  Description of Artificial Sequence Synthetic oligonucleotide
auggcaacacgaguuuacaaauacggucuugugccgcuuggcuacccaccugauucaguggucggaaaaggu

SEQ ID NO: 198 - RNA, 37 nt, Artificial Sequence
  Description of Artificial Sequence Synthetic oligonucleotide
ugcccgcucucuuuuuguugcuaacgugcuguacaug

SEQ ID NO: 199 - RNA, 105 nt, Artificial Sequence
  Description of Artificial Sequence Synthetic polynucleotide
auggcaacacgaguuuacaaauacggucuugugccgcuuggcuacccaccugauucaguggucggaaaagguggugaguuacaaagagcuaauaaucucuggaau

SEQ ID NO: 200 - RNA, 42 nt, Artificial Sequence
  Description of Artificial Sequence Synthetic oligonucleotide
auccucuacgggguguaguaaucgccaauauugggcgagucc

SEQ ID NO: 201 - DNA, 145 bp, Unknown
  Description of Unknown Target sequence
tccatgtctcgttatacgctgtggttcgccaacatcacacgcactacttttggcagacgcagacctacggctacaatggcgaagcgatcctggtattcatgtactacttcacacgcttgacagctagctcagtcctaggtataat

SEQ ID NO: 202 - RNA, 50 nt, Artificial Sequence
  Description of Artificial Sequence Synthetic oligonucleotide
auaacgacccugcgaagugggguguaacuucgacggaucgcuucgccauu

SEQ ID NO: 203 - RNA, 30 nt, Artificial Sequence
  Description of Artificial Sequence Synthetic oligonucleotide
ggaucgcuucgccauuguagccguaggucu

SEQ ID NO: 204 - DNA, 147 bp, Unknown
  Description of Unknown Target sequence
tccatgtctcgttatacgctgtggttcgccaacgcagctaattcactactagcggcttctttttcaacgatgccacccggttggttcagggccggaaccggcttactactgctaattcttgacagctagctcagtcctaggtataat

SEQ ID NO: 205 - DNA, 2774 bp, Artificial Sequence
  Description of Artificial Sequence Synthetic polynucleotide
ccccttgtattactgtttatgtaagcagacaggatgcgtccggcgtagaggatcgagatctccaaaaaatggctgtttttgaaaaaaattctaaaggttgttttacgacagacgataacagggttgaaataattttgtttaactttaagaaggagatttaaatatgaaaatcgaagaaggtaaaggtcaccatcaccatcaccacggatccatgacggcattgacggaaggtgcaaaactgtttgagaaagagatcccgtatatcaccgaactggaaggcgacgtcgaaggtatgaaatttatcattaaaggcgagggtaccggtgacgcgaccacgggtaccattaaagcgaaatacatctgcactacgggcgacctgccggtcccgtgggcaaccctggtgagcaccctgagctacggtgttcagtgtttcgccaagtacccgagccacatcaaggatttctttaagagcgccatgccggaaggttatacccaagagcgtaccatcagcttcgaaggcgacggcgtgtacaagacgcgtgctatggttacctacgaacgcggttctatctacaatcgtgtcacgctgactggtgagaactttaagaaagacggtcacattctgcgtaagaacgttgcattccaatgcccgccaagcattctgtatattctgcctgacaccgttaacaatggcatccgcgttgagttcaaccaggcgtacgatattgaaggtgtgaccgaaaaactggttaccaaatgcagccaaatgaatcgtccgttggcgggctccgcggcagtgcatatcccgcgttatcatcacattacctaccacaccaaactgagcaaagaccgcgacgagcgccgtgatcacatgtgtctggtagaggtcgtgaaagcggttgatctggacacgtatcagtaataaaaagcccgaaaggaagctgagttggctgctgccaccgctgagcaataactagcataaccccttggggcctctaaacgggtcttgaggggttttttgctgaaaggaggaactatatccggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataaca

SEQ ID NO: 206 - DNA, 2771 bp, Artificial Sequence
  Description of Artificial Sequence Synthetic polynucleotide
ccccttgtattactgtttatgtaagcagacaggatgcgtccggcgtagaggatcgagatctccaaaaaatggctgtttttgaaaaaaattctaaaggttgttttacgacagacgataacagggttgaaataattttgtttaactttaagaaggagatttaaatatgaaaatcgaagaaggtaaaggtcaccatcaccatcaccacggatccatggtcagcaagggggaggaagacaatatggctattatcaaggaattcatgcgcttcaaggtgcatatggaaggaagcgtgaatggacacgaattcgagatcgaaggcgagggggagggtcgcccttatgaaggcacacaaacagctaaactgaaagtgacgaagggagggccgcttcccttcgcttgggacattctttcaccccagttcatgtatggttcaaaggcttatgtcaagcacccggcggacattccagactacttaaaattgtcgttccccgaggggtttaaatgggaacgcgttatgaatttcgaggatgggggagtcgtaacggttacccaggacagtagcctgcaggatggcgagttcatctacaaagtgaaattgcgcgggacgaacttccctagcgatgggccagtcatgcagaagaaaacgatgggatgggaagcgtcatccgagcgcatgtatcctgaagatggtgctttaaaaggtgagatcaagcagcgtttgaaactgaaggacgggggccattatgatgctgaagttaaaacgacatataaggccaagaagccagttcaactgccaggggcttataatgttaatattaaattagacattacgagccataatgaagattacacgattgtcgagcaatacgagcgcgcagaaggacgccactcaacggggggcatggacgagctgtacaagtaaaaagcccgaaaggaagctgagttggctgctgccaccgctgagcaataactagcataaccccttggggcctctaaacgggtcttgaggggttttttgctgaaaggaggaactatatccggcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataaca

SEQ ID NO: 207 - RNA, 152 nt, Artificial Sequence
  Description of Artificial Sequence Synthetic polynucleotide
gaaauuaauacgacucacuauagguauaacgacccugcgaagugggguguaacuucgacagagcguaccaucagcuucgaaggcgacggguauaacgacccugcgaagugggguguaacuucgaccuaaccccucucuaaacggagggguuu

SEQ ID NO: 208 - DNA, 30 bp, Unknown
  Description of Unknown Target sequence
agagcgtaccatcagcttcgaaggcgacgg

SEQ ID NO: 209 - RNA, 152 nt, Artificial Sequence
  Description of Artificial Sequence Synthetic polynucleotide
gaaauuaauacgacucacuauagguauaacgacccugcgaagugggguguaacuucgacaaaaacugguuaccaaaugcagccaaaugaguauaacgacccugcgaagugggguguaacuucgaccuaaccccucucuaaacggagggguuu

SEQ ID NO: 210 - DNA, 30 bp, Unknown
  Description of Unknown Target sequence
aaaaactggttaccaaatgcagccaaatga

SEQ ID NO: 211 - RNA, 152 nt, Artificial Sequence
  Description of Artificial Sequence Synthetic polynucleotide
gaaauuaauacgacucacuauagguauaacgacccugcgaagugggguguaacuucgacuagugcagauguauuucgcuuuaaugguacguauaacgacccugcgaagugggguguaacuucgaccuaaccccucucuaaacggagggguuu

SEQ ID NO: 212 - DNA, 30 bp, Unknown
  Description of Unknown Target sequence
tagtgcagatgtatttcgctttaatggtac

SEQ ID NO: 213 - RNA, 152 nt, Artificial Sequence
  Description of Artificial Sequence Synthetic polynucleotide
gaaauuaauacgacucacuauagguauaacgacccugcgaagugggguguaacuucgacugauguggcucggguacuuggcgaaacacuguauaacgacccugcgaagugggguguaacuucgaccuaaccccucucuaaacggagggguuu

SEQ ID NO: 214 - DNA, 30 bp, Unknown
  Description of Unknown Target sequence
tgatgtggctcgggtacttggcgaaacact

SEQ ID NO: 215 - RNA, 152 nt, Artificial Sequence
  Description of Artificial Sequence Synthetic polynucleotide
gaaauuaauacgacucacuauagguauaacgacccugcgaagugggguguaacuucgacugacggcauugacggaaggugcaaaacuguguauaacgacccugcgaagugggguguaacuucgaccuaaccccucucuaaacggagggguuu

SEQ ID NO: 216 - DNA, 30 bp, Unknown
  Description of Unknown Target sequence
tgacggcattgacggaaggtgcaaaactgt

SEQ ID NO: 217 - RNA, 152 nt, Artificial Sequence
  Description of Artificial Sequence Synthetic polynucleotide
gaaauuaauacgacucacuauagguauaacgacccugcgaagugggguguaacuucgacgagcgagacgaaauacgcgaucgcuguuaaguauaacgacccugcgaagugggguguaacuucgaccuaaccccucucuaaacggagggguuu

SEQ ID NO: 218 - DNA, 19 bp, Unknown
  Description of Unknown AAVS1 target locus sequence
gcgagtgaagacggcatgg

SEQ ID NO: 219 - RNA, 53 nt, Artificial Sequence
  Description of Artificial Sequence Synthetic oligonucleotide
auaacgacccugcgaagugggguguaacuucgacgcgagugaagacggcaugg

SEQ ID NO: 220 - RNA, 36 nt, Artificial Sequence; identical to SEQ ID NO: 70
  Description of Artificial Sequence Synthetic oligonucleotide

SEQ ID NO: 221 - protein, 586 aa, Artificial Sequence
  Description of Artificial Sequence Synthetic polypeptide
MATRVYKYGLIPIGYPAKETIDELFKANVLWNNLVALHRKNREDWDDARRAASVLYSDKIDDLEKKEEDLDAAWKAFQQARMDEGTRDETNNKRLKSERASINRLDTEKAEIYKELKPLRKEADKEIDKKQLNDAYRTKVNEAVSVRNSGIYSATAGQILENFKTARDRSFKESATTLRFHRFDGTGYYQFRCRRKGTNVDGISIDDFMSRNFEANPRCAVQSIDNRKKKPRIRIDAVLVGGQSKASKIHQEFDLIYHRPLPIDAQIQNGKILRTRVGDKFKYDLVLTLKIPDKEPISYNNLKGTVGIDIGFRRSVNSLLIGTVMSSDVTEKAYEIKVPPKIVEAFEHVIDLQSELDDAATDLGRIITPLLKAHPLDEDHNKYKMWRSLALRPAHVTLSFEQAYKLAIWLKHETDTFPEEITKKVHTWWRSYSRKYRELHNRRRNQLTHRKHFYREEAAKIVALNKLIVLEEINLTDFAETKEKNTKLSKKARAQRFMASLSEFRDAIRNAAQRDGIGIIDVNPAYTSKTCSECGNLNKDLKSEKQWSCPACGVVHDRDENAANNLQKMGQTYLESLKKETSEVIE

SEQ ID NO: 222 - protein, 585 aa, Artificial Sequence
  Description of Artificial Sequence Synthetic polypeptide
MATRVYKYGLIPIGYPPQAAIDELFRANSLKNTLVALHRESRENWDDARRSASILYSEKMDELDKKNEDITEAFNGLNKARMDEGTKDETGNKRLLAERAIINRLKKEKGDIYAELKPLRKEADKSIDKKALNDAYRQKCNDAVSAKVSGVYRRTAEQIYANFKTAKDKASKDNATLQFHRFDGTGYFQFRCNPKGVSTDGISVDAFMSANFDGYMRCAVQSVDNSKKKPRIRINAVLAGGRTKASKVFQEFDWIYHRPLPADAQIQNGKILRTRVGDKFRYDLVLTIRVPDVEMVQPAKLSGTIGIDVGFRKVGNTLLIGTVMSSDRSQKAVALEVPQMMVSALEHVVALQGELDDAASDLGKAITPLLKANPIDDEHSKYRLWRSLALRPLHVTLSFEQAYKLSLWLKHEPSLFPSEINLKVHTWWRSYSRKYREIHNRRKKQLTHRKHFYRETAAKLVAENKLIVLEDINLTDFAETKSKNTKLSNKARAQRFMASLGEFRDAIKNAAGREGVPVIDVNPAYTSKTCSDCGHLNKELRSEKEWTCPACGVVHDRDENAANNLQKMGQKYLLDVQKAASMVVQ

SEQ ID NO: 223 - protein, 577 aa, Artificial Sequence
  Description of Artificial Sequence Synthetic polypeptide
MIKAFKYGMLEPVAGFDKAAIDVLYLRNKLWNSLVELEKAHRERYRTLITGSDDELSKIQARLDQIEAERAELVKRKRQARAMVRSKKVDTSEHDDRIDMLMAERNDLRTKAKDIRLQVKEKVKPAIADLEKERYEAVKHLIHEAGLWWCNSETVIAAYDLARVKAMKENAELRFRSFDGSGKFAVRKTGGFALSDLVSGKLSFARLEALPDANFAHLSERGKRSRARHHLTMTILTYKDESGKLCRHEVTWPIILHRPLPPEGMIKFIHVQRKRIGKDFQWTCSITMEVDEIQKTPIDHPSRAACGIDIGYRLVKDGLRVAVIADTSGKIDHLTLPQDWIEKMDHVESIQGHLDNSNDLAWGELKALLKSMHDYPESIAESIGRLLKAGDRTPVRGMRALHWRLRNEPETMPEVLSILDTWEAETCRREREMHRLRRKLINRRKDLYRNFAYKVANRYVLIRIRGLSLKKLAAVNLEDGSDNQMPQAVRNNRTRASLSELTLCLQQAAVKAGADFEKVFDVNSTTTCSTCGNQNLKMDREDIYFRCEKCDTLHDQDENAAKNLLRKEFYLAEQAVM

SEQ ID NO: 224 - protein, 582 aa, Artificial Sequence
  Description of Artificial Sequence Synthetic polypeptide
MINCYKFGCLQPTAGFDQSAIEHLFLRNKLWNTLVALDHEFRQRYRDLMLNSDEKLKSVQDSIDSINQEIEDLVENKMKLRQKERTKNIDSKLLDERINVLKAKRKTLSADSKTERERVKVEIKPQIDLLNTERYEAKKLAYKESGLWWGNYETVVAAYDTASQKAMKSNTELRFKSFDGSGKFAVRFEDGGLTIDELKAGASNLCRIETLNTSAFQNLSQRSIKSKARHSLTMTIYTFNDEKGKKQRKEITVPIIFHREMEEGKIKTIHLQRKRLGNQFTWSASFTLKNDIEPANVADHPATASCGIDLGYRLVKDGLRVATVADSQNNVEYLVLPKSWIDRMDYTETLQSGLSEAMTLMWAKLKAEIAKIPEYPDAVAEIIKNMQKMGDRLPYKGIKRLYRVLKEQDATGSPVAGFNAVLDILKAWDKATYRQELEMVNLKDKLLKQREHIYRNFAAGLTKKYAHIVVEDMGLAELAKTEKSETETNDMPNAVKANRQRASLYSLVEAIRLSAAKVGSYFEKSKAAYSSMTCNVCGHLNPKTQNIHQSCESCNTMYDVDENAARNFLKGEYINEKVLKQG

SEQ ID NO: 225 - RNA, 36 nt, Artificial Sequence; identical to SEQ ID NO: 56
  Description of Artificial Sequence Synthetic oligonucleotide

SEQ ID NO: 226 - RNA, 36 nt, Artificial Sequence; identical to SEQ ID NO: 57
  Description of Artificial Sequence Synthetic oligonucleotide

SEQ ID NO: 227 - RNA, 36 nt, Artificial Sequence
  Description of Artificial Sequence Synthetic oligonucleotide
gauuaaggccccugugcaucgggguguaaaugcaac

SEQ ID NO: 228 - RNA, 36 nt, Artificial Sequence
  Description of Artificial Sequence Synthetic oligonucleotide
guugcauuuacaccccgaugcacaggggccuuaauc

SEQ ID NO: 229 - RNA, 26 nt, Artificial Sequence
  Description of Artificial Sequence Synthetic oligonucleotide
gguaucaugacccuacggaugggggg

SEQ ID NO: 230 - RNA, 26 nt, Artificial Sequence
  Description of Artificial Sequence Synthetic oligonucleotide
ccccccauccguagggucaugauacc

SEQ ID NO: 231 - RNA, 34 nt, Artificial Sequence
  Description of Artificial Sequence Synthetic oligonucleotide
aguagaacccugucgcuugggcgguaaagcgaac

SEQ ID NO: 232 - RNA, 34 nt, Artificial Sequence
  Description of Artificial Sequence Synthetic oligonucleotide
guucgcuuuaccgcccaagcgacaggguucuacu

SEQ ID NO: 233 - protein, 7 aa, Artificial Sequence
  Description of Artificial Sequence Synthetic peptide
  MOD_RES (1)..(1): V or A or C
  MOD_RES (2)..(2): Y or F
  MOD_RES (3)..(3): K or Q
  MOD_RES (4)..(4): Y or F
  MOD_RES (6)..(6): L or A or M or C or T
  MOD_RES (7)..(7): I or V or L
XXXXGXX

SEQ ID NO: 234 - protein, 6 aa, Artificial Sequence
  Description of Artificial Sequence Synthetic peptide
  MOD_RES (2)..(2): W or K or R
  MOD_RES (4)..(4): N or T or K or S or D or Q
LXNXLV

SEQ ID NO: 235 - protein, 5 aa, Artificial Sequence
  Description of Artificial Sequence Synthetic peptide
  MOD_RES (3)..(3): G or Y
  MOD_RES (4)..(4): T or S or M
FDXXG

SEQ ID NO: 236 - protein, 7 aa, Artificial Sequence
  Description of Artificial Sequence Synthetic peptide
  MOD_RES (1)..(1): I or L or V
  MOD_RES (2)..(2): Y or L or M or F
  MOD_RES (5)..(5): P or H or D or E
  MOD_RES (6)..(6): L or I or V or M
XXHRXXP

SEQ ID NO: 237 - protein, 7 aa, Artificial Sequence
  Description of Artificial Sequence Synthetic peptide
  MOD_RES (2)..(2): I or L or V
  MOD_RES (4)..(4): I or V or L
  MOD_RES (6)..(6): F or Y
GXDXGXR

SEQ ID NO: 238 - protein, 6 aa, Artificial Sequence
  Description of Artificial Sequence Synthetic peptide
  MOD_RES (2)..(2): K or Q or E
  MOD_RES (3)..(3): H or D or E
  MOD_RES (4)..(4): F or V or L or I
RXXXYR

SEQ ID NO: 239 - protein, 10 aa, Artificial Sequence
  Description of Artificial Sequence Synthetic peptide
  MOD_RES (1)..(1): H or Y
  MOD_RES (3)..(3): R or Q or V
  MOD_RES (5)..(5): E or T or I or H or K or Q or D
  MOD_RES (9)..(9): N or R or I or V or K
XDXDXNAAXN

SEQ ID NO: 240 - protein, 7 aa, Artificial Sequence
  Description of Artificial Sequence Synthetic peptide
  MOD_RES (1)..(1): G or A
  MOD_RES (2)..(2): A or C
  MOD_RES (7)..(7): G or A
XXCCCTX

SEQ ID NO: 241 - protein, 10 aa, Artificial Sequence
  Description of Artificial Sequence Synthetic peptide
  MOD_RES (1)..(1): T or G
  MOD_RES (5)..(5): T or G
  MOD_RES (6)..(6): T or G
  MOD_RES (7)..(7): A or G
  MOD_RES (8)..(8): T or A
  MOD_RES (9)..(9): A or G or C
XGGGXXXXXA

SEQ ID NO: 242 - DNA, 10 bp, Artificial Sequence
  Description of Artificial Sequence Synthetic oligonucleotide
kgggkkrwva

SEQ ID NO: 243 - DNA, 34 bp, Artificial Sequence
  Description of Artificial Sequence Synthetic oligonucleotide
ataacgaccctgcgaagtggggtgtaacttcgac

SEQ ID NO: 244 - DNA, 34 bp, Artificial Sequence
  Description of Artificial Sequence Synthetic oligonucleotide
ttaaggcccctgtctagtggggtgtaactagaac

SEQ ID NO: 245 - DNA, 35 bp, Artificial Sequence
  Description of Artificial Sequence Synthetic oligonucleotide
cttgagaaccctgtcgattgggtggtgaatcgaac

SEQ ID NO: 246 - DNA, 33 bp, Artificial Sequence
  Description of Artificial Sequence Synthetic oligonucleotide
ataggaccctgccgggtggggtgtgacccggac

SEQ ID NO: 247 - DNA, 35 bp, Artificial Sequence
  Description of Artificial Sequence Synthetic oligonucleotide
tttaatgaccctgcgtgttggggtgtgaacacgac

SEQ ID NO: 248 - DNA, 34 bp, Artificial Sequence
  Description of Artificial Sequence Synthetic oligonucleotide
tatctcgaccctacggatggggggaacatccggc

SEQ ID NO: 249 - DNA, 35 bp, Artificial Sequence
  Description of Artificial Sequence Synthetic oligonucleotide
tttaaggaccctgtacgttggggtgtaaacgtaac

SEQ ID NO: 250 - DNA, 33 bp, Artificial Sequence
  Description of Artificial Sequence Synthetic oligonucleotide
taaggcccctgtctagtggggtgtaactagaac